(19) United States
(12) Patent Application Publication
(10) Pub. No.: US 2020/0010850 A1
(43) Pub. Date: Jan. 9, 2020

(54) EFFICIENT CELL FREE PRODUCTION OF PAPILLOMAVIRUS GENE TRANSFER VECTORS

(71) Applicant: The USA, as represeented by the Secretary, Dept. of Health and Human Services, Bethesda, MD (US)

(72) Inventors: John T. SCHILLER, Kensington, MD (US); Carla V. CORREIA CERQUEIRA,, Oxfordshire (GB); Patricia M. DAY, Rockville, MD (US); Douglas R. LOWY, Bethesda, MD (US); David J. FITZGERALD, Rockville, MD (US)

(21) Appl. No.: 16/486,626

(22) PCT Filed: Feb. 20, 2018
(86) PCT No.: PCT/US2018/018735

§ 371 Date: Aug. 16, 2019

Related U.S. Application Data
(60) Provisional application No. 62/460,640, filed on Feb. 17, 2017.

(51) Int. Cl. C12N 15/86; C12N 7/00; C07K 14/21

(52) U.S. Cl. CPC C12N 15/86; C12N 7/00; C07K 14/21; C12N 2710/20042; C12N 2710/20043; C12N 2710/20032; C12N 2710/20023

(57) ABSTRACT

Methods of preparing papillomavirus nucleic acid transfer vectors, in-disassembled cluding by disassembly/reassembly of papillomavirus L1 and L2 virus-like particles, in a defined, cell-free high-efficiency production protocol. These methods may be used to efficiently encapsidate desired moieties, e.g., toxic or therapeutic nucleic acids such as DNA and RNA, and the resultant pseudovirus particles may be used as in vivo delivery vehicles.

### FIELD OF THE INVENTION

This invention relates to cell-free methods for producing papillomavirus pseudovirus particles, and the use of such particles as therapeutic agents.

### BACKGROUND

Papillomaviruses (PVs) are small non-enveloped, circular dsDNA virus. The icosahedral capsid PVs is composed of only two proteins: L1, the major capsid protein and L2, the minor capsid protein. L1 assembles into pentamers and 72 of these capsomers assemble into a T=7 icosahedron. While the L2 protein is mostly located internally it, is essential for infection. It also supports capsid assembly and stabilization, and, depending on the papillomavirus type, is required for DNA packaging (Aydin et al., 2014, PLoS pathogens 10, e1004162.; Chen et al., 2011, Virology 412, 378-83; Day et al., 2004, PNAS USA 101:14252-57; Day et al., 1998, J. Virology 72, 142-50; Holmgren et al., 2005, J. Virology 79, 3938-48; Ishii et al., 2005, Virus Genes 31, 321-28; Kirnbauer et al., 1993, J. Virology 67, 6929-36; Unckell et al., 1997, J. Virology 71, 2934-39; Zhao et al., 1998, Virology 243, 482-91).

The simplicity of PV capsids, and the fact that they can package plasmids up to 8 kB in length that are entirely devoid of PV sequences (Buck et al., 2005b, J. Virology 79:2839-46), make them attractive candidates for gene delivery vectors. Papillomavirus have a unique tissue tropism and mode of infection. They are epitheliotropic, and, in normal tissues, have strict infection tropism for disrupted mucosal and/or cutaneous epithelia (Roberts et al., 2007, Nature Medicine 13:857-86), where they first must bind to specifically modified forms of heparan sulfate proteoglycans on the underlying basement (Johnson et al., 2009, J. Virology 83:2067-74; Kines et al., 2009, PNAS USA 106:20458-63). Papillomavirus pseudovirions (PsV) preferentially transduce epithelial cells and have shown promise as vectors for genetic immunization at cervicovaginal and other mucosal sites in preclinical models. They are particularly adept at inducing long lived, antigen-specific, CD8+, tissue-resident, memory cells in the epithelium infected by the vectors (Cuburu et al., 2012, J. Clin. Invest. 122:4606-20). In addition, it was recently shown that HPV PsV have a strong, and unexpected, tropism for cancer cells (Kines et al., 2016, J. Intl. du Cancer 138:901-11), due to the fact that many cancer cells, particularly carcinomas and melanomas, evolve to express on their surfaces the types of HSPG modifications that are normally found only on the basement membrane. The ability to bind and infect a wide variety of human tumor types, coupled by the inability to bind or infect normal tissue surfaces, make HPV PsVs good candidates for targeted gene delivery into tumor cells for cancer therapy applications.

Efficient generation of PV pseudovirus (PsV) has relied upon both the SV40 origin of replication (ori) being included in the target DNA, and production in cells expressing the SV40 T antigen (generally 293TT cells) (Buck et al., 2004, J. Virology 78:751-57.; Buck and Thompson, 2007, Current protocols in cell biology / editorial board, Juan S Bonifacino [et al] Chapter 26, Unit 26 21; Buck et al., 2005b, supra) to drive intracellular production of a high number of copies of the pseudogenome. However, in such a production system, a fraction of the PsVs encapsidate cellular DNA fragments rather than the target pseudogenome. Next generation sequencing of DNA extracted from PsV preparations produced using the above-described system, has detected incorporation of the T-antigen gene in purified preparations of the particles (Chris Buck and Mike Tisza, personal communications). Since T-antigen is a well-established oncoprotein, this cell culture-dependent PsV production system is unsuitable for generating PV vectors for clinical applications. What is needed is an improved PsV production system that reduces or eliminates the incorporation of contaminating molecules, such as host DNA, into the PsV particles.

Previous work by the inventors has demonstrated that HPV16 L1/L2 virus-like particles (VLPs) are capable of packaging circular plasmids, less that 8 Kb in length, using a cell-free, in-vitro reaction to produce infectious PsV. However, the cell-free system still required that nuclear extract from a mammalian cell, be included in the reaction (Cerqueira et al., 2015, J. Virology 90, 1096-107). The possible presence of contaminants from such extract increase risk that PsVs produced using such a method would fail to meet Good Manufacturing Production (GMP) requirements. Moreover, while the previous work demonstrated that HPV16 PsVs produced by the cell-free method could be used as vectors for clinical gene delivery, repeated application of one type of PsV as a gene transfer vehicle would induce type-restricting, neutralizing antibodies, which would prevent the use of future gene transfer PsVs of the same type. For gene delivery applications, it would be advantageous to have PsV based on other PV types as well.

Thus, it is clear that what is needed is an improved method of producing PsVs, which eliminates contaminants, while producing yields of PsVs sufficient for clinical use. Preferably, such methods would also be applicable to a variety of PsV types. The present invention provides such methods and offers other benefits as well.

SUMMARY

The inventors have recently reported that HPV16 L1/L2 VLPs are capable of packaging circular plasmids, provided they are less that 8 Kb in length, in a cell-free in vitro reaction to generate infectious PsV, but only in the presence of a mammalian cell nuclear extract (Cerqueira, C., Pang, Y. Y., Day, P. M., Thompson, C. D., Buck, C. B., Lowy, D. R., and Schiller, J. T. (2015). A Cell-Free Assembly System for Generating Infectious Human Papillomavirus 16 Capsids Implicates a Size Discrimination Mechanism for Preferential Viral Genome Packaging. Journal of Virology, 90:1096-107). They demonstrated that technology could be used to package a wide range of expression vector plasmids, and therefore that HPV16 pseudovirions (PsVs) produced by those methods could be used as vectors for clinical gene delivery. However, for practical and clinically-useful gene delivery applications, pseudovirion (PsV) based on other PV types are needed. As with the vaccines, application of one type of PsV as a gene transfer vehicle would be expected to induce type-restricted neutralizing antibodies that would prevent gene transfer after additional applications of the same type. Therefore, the inventors extended their studies to other phylogenetically diverse human and animal PV types, while examining the generation of infectious PsV in in vitro reactions using alternative conformations of the pseudogenome, to find efficient packaging protocols for different forms of the plasmid. As described in this disclosure, they have surprisingly determined that highly efficient packaging can be achieved in the absence of a nuclear extract, or any other mammalian cellular components, allowing for HPV pseudovirion production using a GMP-compatible production scheme. An in vivo infection in mouse cervicovaginal challenge model was used to compare PsV produced under the most efficient sets of cell-free reaction conditions with PsV produced by standard intracellular procedures. Their results demonstrate that by manipulating factors such as the presence and concentration of salts, pH, the type of PV from which the L1 and L2 proteins are obtained, and the form of the therapeutic nucleic acid molecules encapsidated, the PsVs produced by the cell-free, “defined” protocols of this disclosure demonstrate comparable infectivity.

Thus, this disclosure provides a method of producing a papillomavirus pseudovirus having an infectivity to particle ratio of at least 1×10⁸ i.u./mg L1 protein, including contacting a virus like particle (VLP) comprising papillomavirus L1 and L2 proteins, with a therapeutic nucleic acid molecule to produce a composition; and, incubating the composition under conditions such that the VLP encapsidates the therapeutic nucleic acid molecule, thereby producing a papillomavirus pseudovirus. The pseudovirus may be incubated with a nuclease with sufficient activity to digest any therapeutic nucleic acid that is not encapsidated. The VLP may be from a human or animal papillomavirus. The VLP may be from an alpha papillomavirus, a beta-papillomavirus, a delta papillomavirus, a gamma papillomavirus, a kappa papillomavirus, or an iota papillomavirus. The VLP may be from an α4, α5, α7, α8, α9, α10, β1, or β2 human papillomavirus. The papillomavirus L1 and L2 proteins may be independently chosen from a HPV type selected from the group consisting of HPV2, HPV5, HPV6, HPV8, HPV16, HPV18, HPV26, HPV31, HPV33, HPV38, HPV39, HPV40, HPV45, HPV52, HPV58, HPV59, HPV68, and animal papillomavirus types MmPV1, BPV1, SfPV1, or MusPV1. The encapsidated therapeutic nucleic acid molecule may be DNA, such as a linear DNA molecule, including linear DNA molecule with blunt ends, or a covalently closed circular DNA molecule, or a nicked closed circular DNA molecule. The therapeutic nucleic acid molecule can be an RNA molecule, including mRNA or a functional RNA, such as, for example, siRNA, shRNA, miRNA, cirRNA, snoRNA, snRNA, piRNA, scaRNA, an aptamer, or a ribozyme.

In an exemplary embodiment, the therapeutic nucleic acid molecule encodes a toxin, which may include an exotoxin, such as abrin, Pseudomonas exotoxin A, diphtheria toxin, cholix toxin, cholera toxin, botulinum toxin, pokeweed antiviral protein, or modified toxins thereof. In one exemplary embodiment, the therapeutic nucleic acid molecule encodes a therapeutic protein, such as a single chain antibody, a cytokine, or a chemokine. In a related embodiment, the therapeutic nucleic acid molecule encodes an antigen which elicits an immune response, thereby acting as a vaccine to immunize a subject against the antigen encoded by the nucleic acid administered within the HPV pseudovirions of this disclosure. Examples of such successful vaccination methodologies are described for the genital herpes pathology by intravaginal vaccination with pseudovirus expressing HSV antigens (see, Cuburu N. J. Virol. 2015; 89:83-96).

In these HPV pseudovirion production methods, the VLP may be contacted with at least 50 ng of the therapeutic nucleic acid per microgram of L1 protein, including between about 50 ng to about 3 micrograms of the therapeutic nucleic acid per microgram L1 protein. The VLP may be contacted with at least 3 micrograms, or more, of the therapeutic nucleic acid per microgram L1 protein.

In these HPV pseudovirion production methods, the composition may comprise less than 600 mM NaCl, or less than 300 mM NaCl, or between 50 mM and 300 mM NaC1, or less than 50 mM NaCl, or from about 0 mM to about 300 mM NaCl, or from about 50 mM to about 300 mM NaCl, or from about 50 mM to about 150 mM NaCl.

In these HPV pseudovirion production methods, the pH of the composition may be in the range of about pH 5.2 to about pH 8.2. In exemplary embodiments, the pH of the composition may be less than or equal to about pH 7.2.

In exemplary embodiments, the pH of the composition less than about pH 6.5. In exemplary embodiments, the pH of the composition less than or equal to pH 6.2. In exemplary embodiments, the pH of the composition less than about pH 5.5. In exemplary embodiments, the pH of the composition less than or equal to pH 5.2. In exemplary embodiments, the pH of the composition less than pH 5.5, and the composition lacks NaCl.

In these HPV pseudovirion production methods, the pH of the composition may alternatively be in the range of about pH 6.0 to about pH 8.2. In exemplary embodiments, the pH of the composition is at least pH 6.0. In exemplary embodiments, the pH of the composition is in the range of about pH 7.2 to about pH 8.2

In exemplary embodiments of the HPV pseudovirion production methods of this disclosure, the composition comprises about 100 mM citrate buffer, pH 5.2, 0.02% Tween 80, and at least 50 ng of the nucleic acid molecule per microgram of L1 protein.

In other exemplary embodiments of the HPV pseudovirion production methods of this disclosure, the pH of the composition is between about pH 7.2 and about pH 8.2, and the composition comprises between about 100 mM and about 150 mM NaCl.

In these methods, the composition may also comprise calcium chloride.

Thus, in other exemplary embodiments of the HPV pseudovirion production methods of this disclosure, the composition comprises 100 mM Tris pH7.2, about 150 mM NaCl, about 10 mM CaCl2, about 0.02% Tween 80, and at least 50 ng therapeutic nucleic acid per microgram of L1 protein.

In aspects of the HPV pseudovirion production methods of this disclosure, prior to contacting the papillomavirus VLP with the therapeutic nucleic acid molecule, the VLP is disassembled. This disassembly may be accomplished by incubation in a solution comprising sodium chloride, a reducing agent, and a detergent. Such solution may comprise between 50 mM NaCl and 200 mM NaCl, at least 2 mM dithithreitol (DTT), and at least 0.01% Tween 80.

In these aspects, the disassembled VLPs are contacted with the therapeutic nucleic acid molecule, and the VLP proteins are reassembled into pseudovirions by contacting the composition with a buffer comprising sodium chloride, calcium chloride, and a detergent. In these methods, the buffer may comprise at least 100 mM sodium chloride, at least 5 mM calcium chloride, and at least 0.01% detergent. For example, the buffer may comprise about 150 mM sodium chloride, about 10 mM calcium chloride, and about 0.02% detergent. In these methods, the pH of the composition may be between about pH 7.2 and about pH 8.2. In these methods, the reassembled pseudovirions may be contacted with oxidized glutathione.

The HPV pseudovirion production methods of this disclosure may include

incubating a papillomavirus VLP in a buffer sufficient to disassemble the papillomavirus VLP and contacting the disassembled papillomavirus VLP with a therapeutic nucleic acid molecule to produce the composition. The composition is then diluted at least 2-fold, at least 4-fold, at least 5-fold, or at least 10-fold to reassemble the pseudovirus, and the pseudovirus is incubated with at least 5 mM oxidized glutathione. In these methods, the pseudovirus may be incubated with a nuclease with sufficient activity to digest any therapeutic nucleic acid that is not encapsidated.

This disclosure also provides a papillomavirus pseudovirion produced according to these methods. This disclosure also provides a method of delivering a therapeutic nucleic acid molecule to a cell, comprising contacting the cell with the pseudoviruses produced by these methods.

In these methods, it may be possible for example to kill a tumor cell, by contacting the tumor cell with a pseudovirion produced by the methods of this disclosure, wherein the pseudovirion comprises a therapeutic nucleic acid molecule that is toxic to the tumor cell, or which encodes a therapeutic protein or therapeutic RNA that is toxic to the tumor cell.

Similarly, certain aspects of the invention comprise treating an individual having a tumor, by administering to the individual a pseudovirion produced by the methods of this disclosure, wherein pseudovirion comprises a nucleic acid molecule that is toxic to the tumor cell, or which encodes a therapeutic protein or therapeutic RNA that is toxic to the tumor cell.

Similarly, it may be possible for example to treat an individual having a disease, comprising administering to the individual a pseudovirion produced by the methods of this disclosure, wherein the pseudovirion comprises a nucleic acid molecule that is effective in treating the disease, or which encodes a therapeutic protein or therapeutic RNA that is effective in treating the disease.

Similarly, the pseudovirions produced by the methods of this disclosure may be used to vaccinate an individual having a disease, or at risk of developing a disease, by administering to the individual a pseudovirion produced by the methods of this disclosure, wherein the pseudovirion comprises an antigen which elicits an immune response in the individual, or a nucleic acid molecule that encodes such antigen, thereby vaccinating the individual against the antigen. In these therapeutic methods, the antigen may be a protein associated with cancer (i.e., a cancer antigen) or an increased risk of cancer. Alternatively or additionally, the antigen may be a viral (such as an Herpes Simplex Virus (HSV) protein), bacterial, fungal, or parasitic protein.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF THE FIGURES

FIG. 1 shows that HPV16 can package linear DNA. Intact or Disassembled HPV16 VLPs were reassembled with a GFP reporter plasmid, with (+) or without (−) nuclear extract. Three types of GFP reporter plasmids were used; circular supercoiled, linearized, or blunt. After reassembly, samples were treated with nucleases and HeLa cells were infected with the reassembled products. The number of infected cells (GFP-positive) were analyzed 72 h post infection by flow cytometry. A representative experiment is shown. The error bars represent the deviation between duplicates.

FIG. 2A shows the disassembly of different PVs. HPV types 16, 45, or 2 were disassembled in 100 mM NaCl, 20 mM Tris, pH 8.2, 2 mM DTT, and 0.01% Tween 80 for 3 h at 37° C. BPV1 was disassembled in 50 mM NaCl, 20 mM Tris, pH 8.2, 2 mM DTT, and 0.01% Tween 80 for 3 h at 37° C. Samples were analyzed by electron microscopy.

Scale bars represent 100 nm. FIG. 2B is a table summarizing the results of the initial survey of cell-free in vitro PsV production across PV types, in which the inventors infected HeLa cells with an equivalent amount of total Li protein for all types, and defined intervals for levels of infectivity. These intervals are defined as: not infectious “−”; infection less than 7%); low infection (“+”; infection greater than 7% but less than 35%); middle infection (“++”; infection greater than 35% but less than 65%); high infection (“+++”; i.e infection greater than 65%).

FIGS. 3A and 3B show the infectivity of HPV16 was intact and HPV45 pseudovirions produced by disassembly and reassembly in the presence of different forms of DNA FIG. 3A shows intact HPV16 packaged with DNA and HPV45 that was disassembled prior to reassembly. Reassembly occurred at the indicated pH and NaCl concentrations for 20 h at 37° C. with 150 ng of GFP plasmid (either circular, circular DNA, or linearized DNA). After nuclease treatment, HeLa cells were infected. Infection was scored 72 h post infection. A representative experiment is shown. The error bars show the deviation between duplicates. FIG. 3B shows the infection by intact HPV16 that was incubated at pH 5.2 with the indicated amounts of linearized GFP plasmid (linear DNA). Previously disassembled HPV45 was incubated at pH 7.2 and 150 mM salt with the indicated amounts of circular or linearized GFP plasmid DNA. Reactions were incubated 20 h at 37° C. and then nuclease treated. Infection and analysis occurred as for FIG. 3A. A representative experiment is shown. The error bars show the deviation between duplicates. FIG. 3C shows the effect of GSSG on reassembly. HPV16 or HPV45 were reassembled as described in Example 2, in the presence or absence of 5 mM GSSG. Pseudovirion infection was analyzed by electron microscopy (upper) or 293TT cells infected (lower) with serial dilutions to determine virus titer (represented as infectious units (i.u.)/ml). Values shown in bar graph are mean for at least three independent experiments ±SD. Scale bars represent 100 nm. FIG. 3D is a table summarizing virus titers achieved following the standard versus disassembly/reassembly protocols of this disclosure, including maturation with oxidized L-Glutathione during reassembly of HPV45 particles.

FIGS. 4A and 4B show electron microscopy of defined reassembled HPV16 and HPV45 vectors. In FIG. 4A, intact HPV16 particles were reassembled at pH 5.2 with a linearized Luc/GFP plasmid for 30 h at 37° C. as described in Example 3. HPV45 was disassembled and then reassembled at pH 7.2, 150 mM NaCl, 10 mM CaCl₂, 0.02% Tween80, with linearized or circular Luc/GFP for 30 h at 37° C. Both HPV16 and HPV45 were then incubated for a further 15 h with 5 mM GSGG. After nuclease treatment, samples were centrifuged over an OPTIPREP™ cushion and the viral fraction was analyzed by electron microscopy. As comparison was included standard HPV16 or HPV45 PsVs produced in cultured cells. Scale bars represent 100 nm. FIG. 4B shows electron Microscopy of different Papillomavirus types after reassembly. Disassembled or intact VLPs of different PV types were reassembled with Luc/GFP reporter gene that was circular or linearized. After reassembly and partial purification over an OPTIPREP™ cushion, samples were analyzed electron microscopy. Scale bars represent 100 nm.

FIGS. 5A-5F show that PsVs prepared by the defined methods of this disclosure have similar antibody neutralization, and use the same entry pathway, as the standard PsVs. (FIGS. 5A and 5B) Reassembled or standard HPV16 PsVs packaging a Luc/GFP plasmid were pre-incubated with dilutions of heparin or HI16.V5 antibody for 1 h on ice. 293TT cells were infected with the pre-incubated virus. The number of infected cells (GFP-positive) was analyzed 72 h post infection by flow cytometry. Mean values for at least three independent experiments ±SD normalized for untreated virus are shown. (FIGS. 5C and 5D) Reassembled or standard HPV45 PsVs were pre-incubated with dilutions of heparin or polyclonal HPV45 serum for 1 h. Infection and analysis was performed as for (top). (FIGS. 5E and 5F) 293TT were infected with the reassembled or standard PsVs in the presence of 10 μM furin inhibitor (dec-RVKR-cmk), 20 mM NH4Cl, 300 nM γ-secretase inhibitor (compound XXI), 10 μM cyclosporin A (CsA), or left untreated. The number of infected cells (GFP-positive) was analyzed 72 h post infection by flow cytometry. (bottom left) Infection with HPV 16, (bottom right) infection with HPV45. Mean values for at least three independent experiments ±SD normalized for untreated cells are shown. “Defined circular” and “defined linear” refers to packaging of a circular or linearized plasmid, respectively.

FIGS. 6A-6H show the effect of heparin and entry inhibitors on HPV26, 39, 58, and MusPV1 infection. FIGS. 6A-6C: Reassembled or standard HPV58 (FIG. 6A), HPV39(FIG. 6B), HPV26 (FIG. 6C) or MusPV1 (FIG. 6D) PsVs packaging a Luc/GFP plasmid were pre-incubated with dilutions of heparin for 1 h on ice. 293TT cells were infected with the pre-incubated virus and analysis was performed as described for FIGS. 5A,5C. FIGS. 6E-6H show 293TT cells infected with the reassembled or standard HPV58 (FIG. 6E), HPV39 (FIG. 6F), HPV26 (FIG. 6G) or MusPV1 (FIG. 6H) PsVs in the presence of 10 μM furin inhibitor (dec-RVKR-cmk), 20 mM NH4Cl, 300 nM y-secretase inhibitor (compound XXI), 10 μM cyclosporin A (CsA), or left untreated. Analysis was performed as described for FIGS. 5E,5F. HPV58 and 26 w either disassembled or intact before the reassembly, and HPV39 and MusPV1 were disassembled before reassembly.

FIGS. 7A-7H show the kinetics of in vivo intravaginal infection. Depoprovera-treated BALB/c mice were treated with nonoxynol-9 prior to infection. 1×10⁷ infectious units HPV16 (FIGS. 7A-7C), HPV45 (FIGS. 7D-7F), HPV58 (FIG. 7G) or 3×10⁶ infectious units HPV26 (FIG. 7H) packaging a Luc/GFP plasmid were inoculated intravaginally. The plasmid was either “circular” or linearized (“linear”). Luciferase expression was measured daily after infection. The average radiance ±SEM is shown. 5 mice per group were used. The legend numbers correspond to the different virus preparations used.

FIG. 8 is a table showing the results of incubating intact particles or reassembling disassembled particles at pH 7.2, in 150 mM NaCl, 10 mM CaCl2, 0.02%, in the presence of mRNA. Samples were treated with RNase cocktail and HeLa or 293TT cells infected with the resulting virus. Infection corresponding to GFP expression was measured 72 h post infection. Cells were considered as infected when number of infected cells was 9.5%. For the differences in infection we considered mean fluorescence intensity (MFI) fold-increase above background and is defined as “+”, MFI<5x; “++”, MFI>5x <20x and “+++” MFI >20x.

FIGS. 9A-9C demonstrate that mRNA PsVs are susceptible to the same entry inhibitors as DNA viruses. FIG. 9A shows the relative infection (as a percentage) of HeLa cells. The indicated viruses packaging GFP mRNA were incubated with 1:500 dilution neutralizing sera or 1 mg/ml heparin before infection of HeLa cells (* Indicates the viruses that were not tested for neutralization sera). For all other inhibitors, HeLa cells were infected with the indicated virus type packaging GFP mRNA in the presence of inhibitors. Inhibitor: NH4Cl—20 mM; γ-sec inhibitor—300 nM γ-sec inhibitor compound XXI; CsA—10 μM cyclosporine A; Aph—3 μM Aphidicolin. GFP expression (infection) was determined at 72 h p.i. by flow cytometry. Represented is the mean of at least three independent normalized against untreated cells, error bars represent the SD (standard deviation). FIG. 9B shows the effect on the ability of leptomycin B to inhibit infection of cells by HPV45 PsVs. HeLa cells were infected with HPV45 PsVs containing GFP-encoding mRNA or DNA in the presence of the indicated concentrations of Leptomycin B. GFP expression (infection) was determined at 72 h p.i. by flow cytometry. Represented is the mean of at least three independent experiment normalized against untreated cells, error bars represent the SD. FIG. 9C demonstrates that leptomycin B by itself, was not toxic to HeLa cells. HeLa cells were infected with Vaccinia Virus—GFP in the presence of Leptomycin B. GFP expression was determined 16 h p.i. by flow cytometry. Represented is the mean of at least three independent experiment normalized against untreated cells, error bars represent the SD.

FIGS. 10A and 10B demonstrate that HPV45 packages PE64 mRNA. HPV45 PsVs coding PE64, PE64Δ4553 or GFP mRNA were prepared using methods of this disclosure at different virus-to-mRNA ratios ranging from 1 to 200 molecules per capsid. HeLa cells were infected with either 50 ng (FIG. 10A) or 10 ng (FIG. 10B) of the resulting virus and cellular viability was measured at 72 h p.i. by XTT assay. Represented is the mean of at least three independent normalized against uninfected cells, error bars represent the SD.

FIG. 11 shows the time course of PE64 killing. HeLa cells were infected with the indicated concentrations of HPV45 PsVs containing PE64 mRNA. Cell death was measured daily using XTT assay. Represented is the mean of at least three independent experiments normalized against uninfected cells, error bars represent the SD.

FIG. 12 shows that cell death is mediated by HPV45 infection and PE64 expression. HeLa cells were infected with HPV45 PsVs packaging PE64 or PE64Δ553 mRNA in the presence of inhibitors. The viruses were pre-incubated with a 1:500 dilution of HPV45 neutralizing sera (neut. sera) or with 1 mg/ml heparin for one hour prior to infection. All other inhibitors were pre-incubated for 30 min before infection and were kept in the culture media throughout the experiment and used at the following concentrations: NH4Cl—20 mM; γ-sec inh—300 nM γ-sec inhibitor compound XXI; CsA—10 μM cyclosporine A; Aph—3 μM Aphidicolin; olaparib—5 μM olaparib; Z-VAD —100 μM Z-VAD-fmk; PE Ab—2 μg/ml PE antibody GFP expression (infection) was determined at 72 h p.i. by flow cytometry. Represented is the mean of at least three trials normalized against uninfected cells, error bars represent the SD.

FIGS. 13A and 13B demonstrate that several papillomaviruses can package PE64 mRNA and induce cell death. The indicated HPV type was produced using the IVP procedures of this disclosure in the presence of the indicated concentrations of capsid to mRNA ratio of PE64 (FIG. 13A) or PE64Δ553 (FIG. 13B) mRNA. HeLa cells were infected with 50 ng of the resulting virus and cellular viability was measured at 72 h p.i. by XTT assay. Represented is the mean of at least three independent trials normalized against uninfected cells, error bars represent the SD.

FIG. 14 demonstrates that HPV45 PE64 mRNA PsVs can kill several tumor cell lines in vitro. HeLa, H460, 4T1 or TC-1 cells were infected with the indicated amounts of HPV45 PE64 mRNA PsVs. Cellular viability was measured at 72h p.i. by XTT assay.

Represented is the mean of at least three independent experiments normalized against uninfected cells, error bars represent the SD.

### DETAILED DESCRIPTION OF THE INVENTION

The inventors have made the surprising discovery that intact, or disassembled/reassembled, papillomavirus VLPs can encapsidate therapeutic nucleic acid molecules, and that such nucleic acid molecules can be totally devoid of papillomavirus genome sequences. Additionally, the inventors have discovered that such therapeutic nucleic acid molecules can be composed of forms, (e.g. linear DNA or RNA) that differ markedly from the double stranded close circular DNA genome of authentic papillomavirus. The inventors have also discovered that the encapsidation of such nucleic acid molecules can be achieved in an in vitro reaction, in the absence of cellular factors and with efficiencies sufficient for production scale up for wide spread clinical uses. The best production efficiencies are achieved under reaction conditions that are unique to the papillomavirus type used, and are not readily predictable between different papillomavirus types. Thus, the present disclosure provides novel papillomavirus pseudovirions, methods of making these papillomavirus pseudovirions, as well as methods of using such pseudovirions for therapeutic applications. In particular, the present disclosure provides novel methods for the efficient, in vitro packaging of therapeutic nucleic acid molecules into papillomavirus virus-like particles (VLPs), yielding pseudovirions capable of efficiently infecting cells.

Accordingly, a method of the present disclosure can generally be practiced by contacting a papillomavirus virus-like particle (VLP) with a therapeutic nucleic acid molecule under conditions suitable for incorporation of the therapeutic nucleic acid molecule into the VLP, yielding a papillomavirus pseudovirus having a high infectivity to particle ratio. In accordance with the present disclosure, preferred conditions utilized in such methods lack cellular factors. In certain embodiments, intact papillomavirus VLPs are contacted with the therapeutic nucleic acid molecule. In certain embodiments, the papillomavirus VLP is disassembled prior to, and reassembled subsequent to, contact with the therapeutic nucleic acid molecule. Suitable conditions for performing such methods are disclosed herein.

Before further describing the invention, it is to be understood that the disclosed invention is not limited to the specific embodiments described or exemplified herein, as such may, of course, vary. It is also to be understood that the terminology used herein is for the purpose of describing particular embodiments only, and is not intended to be limiting, since the scope of the present invention will be limited only by the claims.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

The claims may be drafted to exclude any optional element. As such, this statement is intended to serve as antecedent basis for use of such exclusive terminology as “solely,” “only” and the like in connection with the recitation of claim elements, or use of a “negative” limitation. Further, while various embodiments and technical aspects of the invention may appear in separate locations in the specification, it should be clear that combinations of such embodiments and technical aspects are also encompassed by the invention.

Unless defined otherwise, all technical and scientific terms used herein have the same meaning as is commonly understood by one of skill in art to which the subject matter herein belongs.

The following definitions are supplied to facilitate the understanding of the present invention.

As used herein, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. The terms “comprising,” “including,” and “having” can also be used interchangeably. Furthermore, the phrase “selected from the group consisting of” refers to one or more members of the group in the list that follows, including mixtures (i.e. combinations) of two or more members. As used herein, “at least one” means one or more. The term “comprise” is generally used in the sense of “including”, or “permitting the presence of one or more features or components.” Where descriptions of various embodiments use the term comprising, those skilled in the art will understand that in some specific instances, an embodiment can be alternatively described using the transitional phrase “consisting essentially of”

The present disclosure provides methods for producing a papillomavirus pseudovirus having a high infectivity to particle ratio. As used herein, the term papillomavirus (PV) refers to any member of the family Papillomaviridae, including both human papillomaviruses (HPV) and PVs that infect non-human animals (e.g., mouse PV, bovine, PV, etc.). Preferred papillomaviruses are those against which the majority (e.g., greater than 50%, 60%, 70%, 80%, 90%, or 95%) of a population (e.g., human population), lacks an antibody response, and in particular a neutralizing antibody response. In some aspects, HPVs used to practice the disclosed methods are selected from a Papillomaviridae genus selected from the group consisting of alpha-papillomavirus, beta-papillomavirus, delta-papillomavirus, kappa-papillomavirus, gamma-papillomavirus and iota-papillomavirus. In one aspect, HPVs suitable for practicing methods of the present invention include, but are not limited to, HPV1, HPV2, HPV3, HPV4, HPVS, HPV6, HPV7, HPV8, HPV10, HPV11, HPV16, HPV18, HPV26, HPV31, HPV33, HPV34,

HPV38, HPV39, HPV40, HPV41, HPV42, HPV 43, HPV44, HPV45, HPV51, HPV52, HPV53, HPV54, HPV55, HPV58, HPV59, HPV68, and animal papillomaviruses such as, for example, MusPV1, SfPV1, MmPC1, and BPV1.

As used herein, a therapeutic nucleic acid molecule refers to a nucleic acid molecule having, or encoding a protein or regulatory RNA having, therapeutic, preventative, or toxic activity, and which has been introduced into a VLP production system, with the goal of intentionally packaging the therapeutic nucleic acid molecule within a VLP of this disclosure. The resulting VLP comprising the encapsidated therapeutic nucleic acid molecule is referred to herein as a pseudovirus.

As used herein, a virus-like particle (VLP) is a particle comprising one or more viral capsid, or coat, proteins, which self-assemble into a roughly spherical particle, such that the three-dimensional conformation of the VLP mimics the conformation, and usually the antigenicity, of the authentic native virus from which the capsid, or coat, proteins originate. For ease of discussion, the terms capsid protein and coat protein will be used interchangeably. VLPs of the invention lack nucleic acid sequences that enable autonomous replication of the VLP. That is, upon entry into a cell, the VLP is unable to autonomously initiate or implement the production of VLPs, or virus particles, of the same family of virus from which the VLP capsid proteins originate. Accordingly, VLPs lack the genome of the virus from which the capsid proteins originate. VLPs also typically lack functional nucleic acid sequences encoding functional replicase proteins, or capsid proteins of the virus from which the VLP coat proteins originate. It is understood by those skilled in the art that, generally, VLPs are envisioned as an empty shell made from viral capsid proteins, and which lack any appreciable nucleic acid molecules. However, VLPs may, but need not, contain a small amount of nucleic acid molecules, which are unrelated to the virus from which the VLP capsid proteins originate. As used herein, unrelated nucleic acid molecules are molecules from an organism in a family other than the family of the virus from which the VLP capsid proteins originate. For example, during assembly of a papillomavirus VLP in a human cell, a small amount of host DNA, or RNA, may be packaged within the VLP. The packaged human DNA/RNA would be considered unrelated to papillomavirus. VLPs and methods of producing VLPs are well known to those skilled in relevant arts.

An important aspect of papilloma VLPs, and consequently papilloma pseudovirus particles, is that, due to the fact that their three-dimensional structure mimics that of a native papillomavirus, they are capable of binding to and infecting cells. This feature allows papilloma pseudovirus particles to carry therapeutic nucleic acid molecules into cells.

As used herein, a papillomavirus pseudovirus (PsV) refers to a pseudovirus made from a VLP comprising one or more capsid proteins from a papillomavirus. The terms papillomavirus pseudovirus, papilloma pseudovirus, papilloma pseudovirus particle, and the like, will be used interchangeably herein. Thus, in some aspects, papillomavirus VLPs, and consequently, papilloma pseudoviruses, of the disclosure comprise a papillomavirus L1 protein. In a preferred embodiment, papillomavirus VLPs, and papilloma pseudoviruses, of the disclosure comprise a papillomavirus L1 protein and a papillomavirus L2 protein. The L1 and L2 proteins may, but need not, be wild-type papillomavirus proteins. In some aspects, the L1 and/or L2 proteins can be altered by mutations, including insertions, deletions and substitutions, so that the resulting mutant L1 and/or L2 proteins comprise only the minimal domains, or sequences, essential for assembly of the mutant L1 and/or L2 proteins into papillomavirus VLPs and pseudoviruses capable of infecting cells. In certain aspects, the L1 and L2 proteins comprise an amino acid sequences at least 85%, at least 90%, at least 95%, at least 97% or at least 100% identical to the full-length sequence of a wild-type papillomavirus L1 and/or L2 protein. The use of papillomavirus L1 and/or L2 proteins to produce papillomavirus VLPs is disclosed in U.S. Pat. No. 6,962,777, and U.S. Patent Publication Nos. 2012/0171290, and 2001/0021385, all of which are incorporated herein by reference in their entirety.

As used herein, a therapeutic nucleic acid molecule is a nucleic acid molecule, the introduction of which into a cell results in a therapeutic effect. As used herein, a therapeutic effect refers to clinical improvement in the signs and/or symptoms of a disease or condition. Therapeutic nucleic acid molecules used in the present invention can cause the therapeutic effect directly, or the therapeutic effect can result from transcription or translation of the therapeutic nucleic acid molecule. Accordingly, therapeutic nucleic acid molecules useful for practicing the present invention include, but are not limited to, DNA molecules, RNA molecules, including mRNA and functional RNA molecules, modified version thereof, and mixtures thereof. Therapeutic nucleic acid molecules may encode proteins such as toxins, cancer associated antigens, or viral, bacterial, fungal, or parasitic antigenic proteins, which effectively vaccinate an individual against the antigenic protein. Alternatively therapeutic nucleic acids may be DNAs encoding functional (i.e., regulatory) RNAs, or they may be functional RNAs themselves.

Therapeutic nucleic acid molecules of the present disclosure can, but need not, comprise intronic sequences, and thus, this term encompasses open-reading frames (ORFs). Moreover, this term can, but need not, encompass control elements functionally linked to the nucleotide sequences. As used herein, the phrase “functionally linked” means the control element directs and/or regulates (e.g., initiates, suppresses, etc.) transcription of the nucleotide sequence. Examples of such control elements include, but are not limited to, promoter sequences, enhancer sequences, and repressor sequences.

In one aspect, a therapeutic nucleic acid molecule can be a DNA molecule. Such DNA molecules can comprise a nucleotide sequence, the transcription of which results in production of a therapeutic protein or a therapeutic RNA molecule. DNA molecules can include linear DNA molecules, such as PCR products and linearized plasmids, intact plasmids and viral vectors. As used herein, a viral vector refers to a nucleic acid molecule constructed, in part, from viral genomic DNA, and which therefore comprises at least a portion of a viral genome, and which comprises a gene of interest (e.g. a nucleic acid molecule encoding a protein). Viral nucleotide sequences (i.e., sequences from the viral gnome) in such a vector may be useful for producing large quantities of the viral vector in cell culture, which are then used in the production methods disclosed herein. However, such viral sequences are not necessary for practicing the methods disclosed herein. Viral vectors suitable for practicing the invention are known to those skilled in the art and include, but are not limited to, adenovirus vectors, AAV vectors, baculovirus vectors, lentivirus vectors, herpesvirus vectors, alphavirus vectors and retrovirus vectors.

A therapeutic protein of the invention is a protein which, when produced in a cell, produces a desired, therapeutic effect. The therapeutic protein can act within the cell in which it is expressed (e.g., regulate or inhibit transcription of a gene), or it may be secreted from the cell in which it is expressed and act at a distant site (e.g., bind a receptor at a distant site, or elicit an immune response in the individual to whom the pseudovirion is administered). Examples of therapeutic proteins include, but are not limited to, regulators of transcription, tumor suppressor proteins, pro-apoptotic proteins, suicide proteins, cytokines, lymphokines, monokines, hormones, growth factors, enzymes, immunomodulatory proteins, toxins, cytotoxins, pro-drugs, cancer antigens, antigenic viral proteins, antigenic bacterial proteins, antigenic fungal proteins, antigenic parasitic proteins, and modified or variant forms thereof. Examples of cytotoxins include, but are not limited to, the exotoxins abrin, Pseudomonas exotoxin A, diphtheria toxin, cholix toxin, cholera toxin, botulinum toxin, pokeweed antiviral protein, CelTOS, and modified toxins thereof. The cytotoxin can be modified into a form for use as an immunotoxin by removing the native targeting component of the toxin (e.g., domain Ia of PE or the B chain of DT) and replacing it with a different targeting moiety, such as an antibody. See, e.g., Kreitman, The AAPS Journal (2006) 8(3):E532-551 and the references cited therein. Preferred toxins inhibit protein synthesis, e.g., are ADP-ribosylating agents or ribosomal inactivating agents. On such toxin is Pseudomonas exotoxin.

Pseudomonas exotoxin (PE) is a three domain bacterial toxin expressed by most strains of Pseudomonas aeruginosa. It is considered to be a cytotoxic virulence factor during Pseudomonas infections and may cause damage to host tissue either systemically or locally, depending on the nature of the infection. PE inhibits protein synthesis of eukaryotic cells by ADP-ribosylation of elongation factor-2 (EF2) which leads to the cessation of translation. The enzymatic domain occupies the C-terminal portion of the toxin. The binding and entry domains are located at the N-terminus and middle, respectively. At the extreme C-terminus, a KDEL-like sequence is present that aids intracellular trafficking, presumably involving elements of the ER. After surface binding to either low density lipoprotein receptor-related protein 1a or b (LRP1a or LRP1b), the toxin enters cells, is processed by furin and traffics to the ER via retrograde transport. The translocation step from the ER to the cytosol is not well understood but may involve ER-associated protein degradation (ERAD), the quality control pathway within the ER. PE has been used extensively to generate antibody-toxin (immunotoxins) therapeutic proteins for cancer. This is typically accomplished by substituting the toxin's cell binding domain with an antibody fragment that selectively recognizes and binds to cancer cells. The toxin is a potent cell-killing agent and is currently undergoing evaluation in several clinical trials. However, a potential hindrance to the efficacy of immunotoxins is the generation of neutralizing antibodies by patients who receive antibody-toxin treatments. The use of non-immunogenic mRNA encoding the toxin could overcome this problem, if the RNA can be delivered efficiently and by a non-immunogenic route.

As used herein, the term Pseudomonas exotoxin A (“PE”) as used herein includes reference to forms of PE which have been modified but which retain cytotoxic function. Thus, the PE molecule can be truncated to provide a fragment of PE which is cytotoxic but which does not bind cells, as in the fragments known as PE38 and PE40, or can have mutations which reduce non-specific binding, as in the version called “PE4E”, in which four residues are mutated to glutamic acid. Further, a portion of the PE sequence can be altered to increase toxicity, as in the form called “PE38 KDEL”, in which the C-terminal sequence of native PE is altered, or the form of PE discussed herein, in which the arginine corresponding to position 490 of the native PE sequence is replaced by alanine, glycine, valine, leucine, or isoleucine.

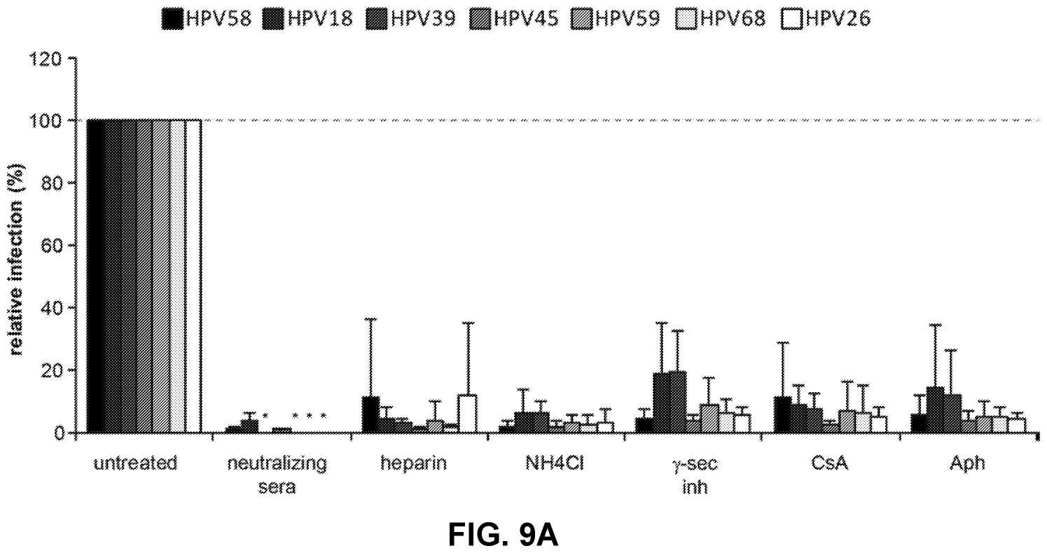

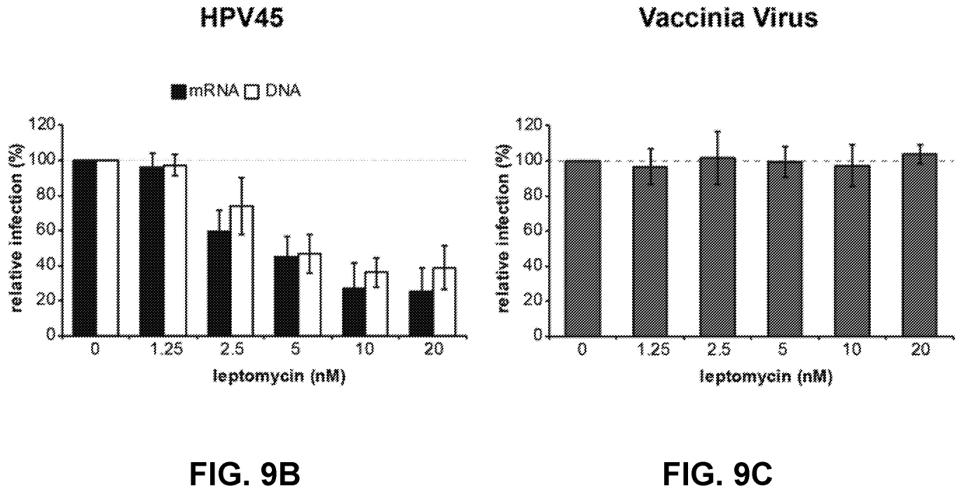

As used herein, the term “Cholix toxin” (“CT”), refers to a toxin expressed by some strains of Vibrio cholerae that do not cause cholera disease. According to the article reporting the discovery of the Cholix toxin (Jorgensen, R. et al., J Biol Chem. 283(16):10671-10678 (2008)), mature cholix toxin is a 70.7 kD, 634 residue protein (see FIG. 9C of PCT/US2009/046292, which is incorporated herein by reference). The Jorgensen authors deposited in the NCBI Entrez Protein database a 642-residue sequence which consists of what they termed the full length cholix toxin A chain plus, at the N-terminus an additional 8 residues, consisting of a 6 histidine tag flanked by methionine residues, presumably introduced to facilitate expression and separation of the protein. The 642-residue sequence is available on-line in the Entrez Protein database under accession number 2Q5T_A and can be converted to the 634 amino acid sequence by simply deleting the first 8 amino acids of the deposited sequence. Mature CT has four domains: Domain Ia (amino acid residues 1-269), Domain II (amino acid residues 270-386), Domain Ib (amino acid residues 387-415), and Domain III (amino acid residues 417-634).

As used herein, the term “Cholera exotoxin” (“CET”) refers to a toxin expressed by some strains of Vibrio cholerae that do not cause cholera disease and include mature CET and cytotoxic fragments thereof. Mature cholera exotoxin (CET) is a 634 amino acid residue protein whose sequence is set forth as in FIG. 9C of PCT/US2009/046292. For convenience of reference, the terms “cholera exotoxin,” and “CET” as used herein may refer to the native or mature toxin, but more commonly refer to forms in which the toxin has been modified to reduce non-specific binding, for example, by deletion of domain Ia, or otherwise improve its utility for use in immunotoxins. A CET protein may be a full-length CET protein or it may be a partial CET protein comprising one or more subdomains of a CET protein and having cytotoxic activity as described herein. Mature CET has four domains: Domain Ia (amino acid residues 1-269), Domain II (amino acid residues 270-386), Domain Ib (amino acid residues 387-415), and Domain III (amino acid residues 417-634). CelTos, refers to the full-length malarial protein (Kariu et al. Molec Microbiology 2006; 59:1369-1379) and amino acid deletion and substitution variants that retain the ability to form pores in mammalian cell membranes.

The inventors have surprisingly discovered that papillomavirus VLPs can also package RNA molecules. Thus, in one aspect, the therapeutic nucleic acid molecule can be a therapeutic RNA molecule. As used herein, a therapeutic RNA molecule is an RNA molecule that when introduced into a cell, results in a desired therapeutic effect., Examples of a therapeutic RNAs include, but are not limited to, messenger RNAs (mRNA) and functional RNAs. In one aspect, the therapeutic nucleic acid molecule is an mRNA molecule encoding a therapeutic protein. Examples of useful therapeutic proteins have been disclosed herein. In one aspect, the therapeutic RNA is a functional RNA. Examples of functional RNA molecules include, but are not limited to, small interfering RNA (siRNA) molecules, short hairpin RNA (shRNA) molecules, micro RNA (miRNA) molecules, circular RNA (cirRNA) molecules, small nucleolar RNA (snoRNA) molecules, small nuclear ribonucleic acid RNA (snRNA) molecules, piwi-interacting RNA (piRNA) molecules, small Cajal body RNA (scaRNA) molecules, aptamers, ribozymes, and the like. Examples of such therapeutic RNAs are disclosed in U.S. Pat. No. 8,987,225, which is incorporated herein by reference.

Methods of the present disclosure produce papillomavirus pseudovirions having high infectivity to particle ratios. An infectivity to particle ratio represents the infective ability (e.g. efficiency) of a papillomavirus pseudovirus, in that it is a measure of the amount of pseudovirus needed to cause infection of a cell. Accordingly, a first papillomavirus pseudovirus having an infectivity to particle ratio that is higher than infectivity to particle ratio of a second papillomavirus pseudovirus, is more efficient than the second papillomavirus pseudovirus at infecting a cell. Infectivity of papillomavirus pseudovirus particles can be determined using techniques known in the art. For example, infection of cells by papillomavirus pseudovirions carrying a gene encoding a fluorescent protein can be determined by scanning cells for fluorescence from the fluorescent protein. Such determination can be made using, for example, a fluorescence cell sorter/counter, or by imaging cells of cells grown in culture, or counting fluorescent cells in a tissue culture dish. Infection of cells by papilloma pseudovirus particles of the invention can also be determined using other labels, such as, for example, expression of luminescent proteins, and immunogenic proteins (e.g., epitope tagging). In the present disclosure, the level of infectivity is reported as infectious units.

In methods of the present disclosure, infectious units are reported relative to a specific amount of papillomavirus pseudovirus. The amount of papillomavirus pseudovirus can be measured using techniques known in the art. For example, the amount of papillomavirus pseudovirus can be determined physically by the number of particles in a sample using techniques such as electron microscopy scanning, or fluorescent sorting of particles that bind papillomavirus pseudovirus particles. In certain method of the invention, the amount of papillomavirus pseudovirus particles is based on the amount (e.g., in milligrams (mg)) of virion protein present in a sample. Thus, in one aspect, the infectivity to particle ratio is measured as infectious units per milligram of L1 protein (i.u./mg L1).

The ability of papillomavirus pseudovirus particles to infect a cell is based, at least in part, on the three-dimensional conformation of capsid proteins in the papillomavirus pseudovirus particles. Thus, the infectivity to particle ratio may provide an indication of how accurately the three-dimensional conformation of capsid proteins in the papillomavirus pseudovirus particles mimics the three-dimensional conformation of coat protein in native papillomavirus particles. Preferred methods are those producing papillomavirus pseudovirus particles having a high infectivity to particle ratio. As used herein, a high infectivity to particle ratio is a ratio which is at least 10%, at least 20%, at least 30%, at least 40%, at least 50%, at last 60%, at least 70%, at least 80%, or at least 90%, of the infectivity to particle ratio of a native papillomavirus of the same type (e.g., HPV4, HPV10, HPV16, etc.). In certain aspects, a high infectivity to particle ratio is at least 10%, at least 20%, at least 30%, at least 40%, at least 50%, at last 60%, at least 70%, at least 80%, or at least 90%, of the infectivity to particle ratio of a papillomavirus pseudovirus of the same type (e.g., HPV4, HPV10, HPV16, etc.), produced using methods currently known in the art. Examples of such currently known methods are disclosed in U.S. Pat. No. 6,962,777.

One aspect of the invention is a method to produce a papillomavirus pseudovirus having a high infectivity to particle ratio, comprising:
a. contacting a papillomavirus virus-like particle (VLP) with a therapeutic nucleic acid molecule to produce a composition; and,
b. incubating the composition under conditions suitable for encapsidation of the therapeutic nucleic acid molecule by the VLP;
   wherein the incubation conditions yield a papillomavirus pseudovirus having a high infectivity to particle ratio.

In one aspect, the papillomavirus VLP is produced using capsid proteins from an alpha-papillomavirus, a beta-papillomavirus, or a gamma-papillomavirus. In one aspect, the papillomavirus VLP is produced using capsid proteins from a α4, α5, α7v, α8, α9, α4, α10, β1or β2 papillomavirus. In one aspect, the papillomavirus pseudovirus comprises a papillomavirus L1 protein. The papillomavirus L1 protein can be at least 85%, at least 90%, at least 95%, at least 97% or 100% identical to an L1 protein from a papillomavirus selected from the group consisting of HPV1, HPV2, HPV3, HPV4, HPV5, HPV6, HPV7, HPV8, HPV10, HPV11, HPV16, HPV18, HPV26, HPV31, HPV33, HPV34, HPV38, HPV39, HPV40, HPV 41, HPV 42, HPV 43, HPV 44, HPV45, HPV51, HPV52, HPV53, HPV54, HPV55, HPV58, HPV59, HPV68, MmPV1, BPV1, SfPV1, and MusPV1. In one aspect, the papillomavirus pseudovirus comprises a papillomavirus L1 protein and a papillomavirus L2 protein. The papillomavirus L2 protein can be at least 85%, at least 90%, at least 95%, at least 97% or 100% identical to an L2 protein from a papillomavirus selected from the group consisting of HPV1, HPV2, HPV3, HPV4, HPV5, HPV6, HPV7, HPV8, HPV10, HPV11, HPV16, HPV18, HPV26, HPV31, HPV33, HPV34, HPV38, HPV39, HPV40, HPV 41, HPV 42, HPV 43, HPV 44, HPV45, HPV51, HPV52, HPV53,

HPV54, HPV55, HPV58, HPV59, HPV68, MmPV1, BPV1, SfPV1, and MusPV1. In one aspect, the papillomavirus L1 and L2 proteins are independently chosen from one or more papillomaviruses. In one aspect, the papillomavirus L1 and L2 proteins are independently chosen from one or more papillomaviruses selected from the group consisting of HPV1, HPV2, HPV3, HPV4, HPV5, HPV6, HPV7, HPV8, HPV10, HPV11, HPV16, HPV18,

HPV26, HPV31, HPV33, HPV34, HPV38, HPV39, HPV40, HPV 41, HPV 42, HPV 43, HPV 44, HPV45, HPV51, HPV52, HPV53, HPV54, HPV55, HPV58, HPV59, HPV68, MmPV1, BPV1, SfPV1, and MusPV1.

The therapeutic nucleic acid molecule may be functional RNA, or it may encode a protein, or functional RNA.

The therapeutic nucleic acid molecule may be a nucleic acid molecule, such as a DNA molecule. The DNA molecule can be a linear DNA molecule having overhanging ends or having blunt ends. The DNA molecule can also be a covalently closed, circular DNA molecule, such as a plasmid. In certain aspects, one or more strands of the covalently closed, circular DNA molecule has been nicked with a nuclease to prevent super-coiling of the DNA molecule. In certain aspects, the DNA molecule encodes a protein selected from a tumor suppressor protein, a pro-apoptotic protein, a protein that causes cell death, a cytokine, a lymphokine, a , a growth factor, an enzyme, an immunomodulatory protein, a cytotoxin, a pro-drug and a single-chain antibody. In one aspect the encoded protein is a cytotoxin. The cytotoxin may be selected from the group consisting of abrin, Pseudomonas exotoxin A, diphtheria toxin, cholix toxin, cholera toxin, botulinum toxin, pokeweed antiviral protein, and modified or variant forms of these toxins.

The therapeutic nucleic acid molecule may be an RNA molecule. The RNA molecule can be a mRNA molecule, or it can be a functional RNA molecule. Proteins encoded by the mRNA molecule may include tumor suppressor proteins, pro-apoptotic proteins, a protein that causes cell death, cytokines, lymphokines, monokines, growth factors, enzymes, immunomodulatory proteins, cytotoxins, pro-drugs, or single-chain antibodies, antigens such as cancer -associated antigens, viral, bacterial, fungal, or parasitic antigens. Cytotoxins encoded by the RNA molecule may be selected from the group consisting of abrin, Pseudomonas exotoxin A, diphtheria toxin, cholix toxin, cholera toxin, botulinum toxin, pokeweed antiviral protein, and modified toxins thereof.

In one aspect, the RNA is a functional RNA. The functional RNA can be selected from the group consisting of siRNA, shRNA, miRNA, cirRNA, snoRNA, snRNA, piRNA, scaRNA, aptamers, and ribozymes.

In one aspect, the papillomavirus pseudovirus particle has an infectivity to particle ratio that is at least 10%, at least 20%, at least 30%, at least 40%, at least 50%, at last 60%, at least 70%, at least 80%, or at least 90%, of the infectivity to particle ratio of a native papillomavirus of the same type (e.g., HPV16). In certain aspects, a “high” infectivity to particle ratio is at least 10%, at least 20%, at least 30%, at least 40%, at least 50%, at last 60%, at least 70%, at least 80%, or at least 90%, of the infectivity to particle ratio of a papillomavirus pseudovirus of the same type, produced using methods currently known in the art. The papillomavirus pseudovirus may have an infectivity to particle ratio of at least 1×10⁸ i.u./mg L1 protein, at least 5×10⁸ i.u./mg L1 protein, at least 1 x 10⁹ i.u./mg L1 protein, at least 5×10⁹ i.u./mg L1 protein, at least 1×10¹⁰ i.u./mg L1 protein, at least 5 x 10¹⁰ i.u./mg L1 protein, or at least 1×10¹⁰ i.u./mg L1 protein.

The present inventors have discovered that the amount of therapeutic nucleic acid molecule contacted with the papillomavirus VLP (as measured in micrograms (ug) of L1 protein) affects the resulting yield of papillomavirus pseudovirus particles. Thus, in one aspect, the papillomavirus VLP is contacted with at least 50 ng of a therapeutic nucleic acid molecule/ug L1 protein, at least 100 ng of a therapeutic nucleic acid molecule/ug L1 protein, at least 50 ng of a therapeutic nucleic acid molecule/ug L1 protein, at least 100 ng of a therapeutic nucleic acid molecule/ug L1 protein, at least 250 ng of a therapeutic nucleic acid molecule/ug L1 protein, at least 500 ng of a therapeutic nucleic acid molecule/ug L1 protein, at least 750 ng of a therapeutic nucleic acid molecule/ug L1 protein, at least 1 ug of a therapeutic nucleic acid molecule/ug L1 protein, at least 1500 ng of a therapeutic nucleic acid molecule/ug L1 of a therapeutic nucleic acid molecule/ug L1 protein, at least 2 ug of a therapeutic nucleic acid molecule/ug L1 protein, at least 2500 ng of a therapeutic nucleic acid molecule/ug L1 protein, or at least 3 ug of a therapeutic nucleic acid molecule/ug L1 protein. Thus, in one aspect, the papillomavirus VLP is contacted with an amount of nucleic acid molecules in the range of at least 50 ng of a therapeutic nucleic acid molecule/ug L1 protein to at least 3 ug of a therapeutic nucleic acid molecule/ug L1 protein.

It is understood in the art that, due to ionic and non-ionic interactions, nucleic acid molecules, may adhere to the outside of a papillomavirus pseudovirus particle, instead of being encapsidated within the particle. Thus, to insure that the only therapeutic nucleic acid molecules associated with the papillomavirus pseudovirus are encapsidated therapeutic nucleic acid molecules, the papillomavirus pseudovirus particles are optionally further treated with a nuclease to remove nucleic acid molecules that are not encapsidated in the papillomavirus pseudovirus particles, i.e., nucleic acid molecules associated with the outside of a papillomavirus pseudovirus particle. Thus, pseudovirus production methods of this disclosure, following incubation of the composition comprising the papillomavirus pseudovirus particle and the therapeutic nucleic acid molecule, the resulting papillomavirus pseudovirus particles may be contacted with a nuclease, such as an exonuclease. Contact of the papillomavirus pseudovirus particles should be under conditions in which the nuclease is of sufficient activity, and the contact sufficiently long, that any non-encapsidated therapeutic nucleic acid molecule is digested. Appropriate nucleases and incubation conditions are known to those skilled in the art.

While previous work in the field has attempted to demonstrate encapsidation of therapeutic nucleic acid molecule by papillomavirus VLPs, the inventors have discovered that the conditions used for such encapsidation can significantly affect the yield of particles produced, and, in particular, the infectivity to particle ratio of the resulting papillomavirus pseudovirus particles. Specifically, the inventors have elucidated conditions for producing papillomavirus pseudovirus particles having high infectivity to particle ratios. More specifically, the inventors have found that by manipulating factors such as the presence and concentration of salts, pH, the type of PV from which the L1 and L2 proteins are obtained, and the form of the therapeutic nucleic acid molecule, papillomavirus pseudovirus particles can be produced that have a infectivity to particle ratio only slightly less than, equal to, or even higher than, the infectivity to particle ratio of native papillomavirus particles, or papillomavirus pseudovirus particles produced using in vivo production methods currently known it the art.

In one aspect, the composition comprising the papillomavirus pseudovirus particle and the therapeutic nucleic acid comprises less than about 600 mM NaCl, less than about 300 mM NaCl, less than about 200 mM NaCl, less than about 100 mM NaCl, or less than about 50 nM NaCl.

In certain aspects, the composition comprising the papillomavirus pseudovirus particle and the therapeutic nucleic acid, comprises an amount of NaCl in a range of from about 0 mM NaCl to about 300 mM NaCl. In one aspect, the composition comprises at least about 50 mM NaCl. In one aspect, the composition comprises an amount of NaCl in a range of from about 50 mM to about 300 mM. In one aspect, the composition comprises in NaCl an amount of from about 50 mM to about 150 mM.

In one aspect, the pH of the composition comprising the papillomavirus pseudovirus particle and the therapeutic nucleic acid molecule is in the range of about pH 5.2 to about pH 8.2 In one aspect, the pH is less than, or equal to, about pH 7.2, less than, or equal to, about pH 6.5, less than, or equal to, about pH 6.2, less than, or equal to, about pH 5.5, or less than, or equal to, about pH 5.2.

In certain aspects, the pH of the composition comprising the papillomavirus pseudovirus particle and the therapeutic nucleic acid molecule, is in the range of about pH 6.0 to about pH 8.2. In one aspect, the pH of the composition is at least 6.0. In one aspect, the pH of the composition is in the range of about pH 7.2 to about 8.2.

In one aspect, the pH of the composition comprising the papillomavirus pseudovirus particle and the therapeutic nucleic acid molecule, is less than about pH 5.5, and lacks a detectable level of NaCl. In one aspect, the composition comprising the papillomavirus pseudovirus particle and the therapeutic nucleic acid molecule, has a pH of about pH 5.2, and comprises about 50-150 mM of a buffer, about 0.02% of a surfactant, and at least 50 ng of a therapeutic nucleic acid molecule per microgram of L1 protein. The composition can comprise about 100 mM of a buffer. The buffer can be selected from the group consisting of citrate buffer, tris(hydroxymethyl)aminomethane (Tris, Trizma), 4-Morpholineethanesulfonic acid (MES), Bis(2-hydroxyethyl)amino-tris(hydroxymethyl)methane (Bis-Tris), N-(Carbamoylmethyl)iminodiacetic acid (ADA), N-(2-Acetamido)-2-aminoethanesulfonic acid (ACES), 1,4-Piperazinediethanesulfonic acid (PIPES), β-Hydroxy-4-morpholinepropanesulfonic acid (MOPSO), N,N-Bis(2-hydroxyethyl)-2-aminoethanesulfonic acid (BES), 3-(N-Morpholino)propanesulfonic acid (MOPS), 2-[(2-Hydroxy-1, 1-bis(hydroxymethyl)ethyl)amino]ethanesulfonic acid (TES), 4-(2-Hydroxyethyl)piperazine-1-ethanesulfonic acid (HEPES), 3-(N,N-Bis[2-hydroxyethyl]amino)-2-hydroxypropanesulfonic acid (DIPSO), 4-(N-Morpholino)butanesulfonic acid (MOBS), 2-Hydroxy-3-[tris(hydroxymethyl)methylamino]-1-propanesulfonic acid (TAPSO), 4-(2-Hydroxyethyl)piperazine-1-(2-hydroxypropanesulfonic acid) Hydrate (HEPPSO), and Piperazine-1,4-bis(2-hydroxypropanesulfonic acid) dehydrate (POPSO). The surfactant can be a non-ionic surfactant, such as, for example, Tween-80 or Triton X-100. In one aspect, the composition comprising the papillomavirus pseudovirus particle and the therapeutic nucleic acid molecule, has a pH of about pH 5.2, and comprises about 100 mM of citrate buffer, about 0.02% of Tween-80, and at least 50 ng of the therapeutic nucleic acid molecule per microgram of L1 protein, at least 250 ng of the therapeutic nucleic acid molecule/ug L1 protein, at least 500 ng of the therapeutic nucleic acid molecule/ug L1 protein, at least 750 ng of the therapeutic nucleic acid molecule/ug L1 protein, at least 1 ug of the therapeutic nucleic acid molecule/ug L1 protein, at least 1500 ng of the therapeutic nucleic acid molecule/ug L1 protein, at least 2 ug of the therapeutic nucleic acid molecule/ug L1 protein, at least 2500 ng of the therapeutic nucleic acid molecule/ ug L1 protein, or at least 3 ug of the therapeutic nucleic acid molecule/ug L1 protein. Thus, in one aspect, the papilloma VLP is contacted with an amount of nucleic acid molecules in the range of at least 50 ng of the therapeutic nucleic acid molecule/ug L1 protein to at least 3 ug of the therapeutic nucleic acid molecule/ug L1 protein.

In one aspect, the composition comprising the papillomavirus pseudovirus particle and the therapeutic nucleic acid molecule, has a pH in the range of about pH 7.2 to about pH 8.2, and comprises an amount of NaCl in the range of about 100 mM to about 150 mM. The composition can further comprise a calcium salt, such as, for example, calcium chloride. In one aspect, the composition comprises a non-ionic surfactant, such as, Tween-80 or Triton X-100. In one aspect, the composition comprising the papillomavirus pseudovirus particle and a therapeutic nucleic acid molecule, has a pH in the range of about pH 7.2 to about pH 8.2, and comprises about 50-150 mM of a buffer, about 50 mM -150 mM of NaCl, about 0.02% of a surfactant, about 5 mM-20 mM of a calcium salt, and at least 50 ng of a therapeutic nucleic acid molecule per microgram of L1 protein. In one aspect, the composition comprising the papillomavirus pseudovirus particle and the therapeutic nucleic acid molecule, has a pH of about pH 7.2, and comprises about 100 mM Tris buffer, about 150 mM NaCl, about 0.02% Tween-80, and at least 50 ng of the therapeutic nucleic acid molecule per microgram of L1 protein. The composition can comprise at least 250 ng of the nucleic acid molecule/ug L1 protein, at least 500 ng of the nucleic acid molecule/ug L1 protein, at least 750 ng of the nucleic acid molecule/ug L1 protein, at least 1 ug of the nucleic acid molecule/ug L1 protein, at least 1500 ng of the nucleic acid molecule/ug L1 protein, at least 2 ug of the nucleic acid molecule/ug L1 protein, at least 2500 ng of the nucleic acid molecule/ug L1 protein, or at least 3 ug of the nucleic acid molecule/ug L1 protein. Thus, in one aspect, the papillomavirus VLP is contacted with an amount of therapeutic nucleic acid molecule in the range of at least 50 ng of the nucleic acid molecule/ug L1 protein to at least 3 ug of the nucleic acid molecule/ug L1 protein.

In preferred embodiments, encapsidation of the therapeutic nucleic acid molecule by the VLP is performed under conditions that lack cellular factors. As used herein, the phrase cellular factors means molecules, such as proteins, lipids, carbohydrates, and the like, which are normally found in mammalian cells. Accordingly, cellular factors can refer to extract of mammalian cells or purified molecules from mammalian cells. Those skilled in the art will understand that minute amounts of such factors might be present as contaminates in purified VLPs or nucleic acid molecules. However, it should also be understood that encapsidation of the therapeutic nucleic acid molecule by the VLP is achieved without need of proteins, lipids, carbohydrates, and the like, from mammalian cells. In preferred methods, the incubation conditions lack a biologically active amount of cellular factors.

The inventors have made the novel and surprising discovery that intact papillomavirus VLPs can encapsidate therapeutic nucleic acid molecules. Such nucleic acid molecules can be totally devoid of papillomavirus genome sequences, and can be composed of forms, (e.g. linear DNA or RNA) that differ markedly from the double stranded close circular DNA genome of authentic virus. Such packaging can be achieved in an in vitro reaction, in the absence of cellular factors.

As used herein, an intact papillomavirus VLP is one that has been purified from the cell in which it was produced, and which has not been subjected to conditions (e.g., ionic concentrations, pH, detergent, etc.) sufficient to cause disassembly of the VLP. Thus, the three-dimensional structure of an intact VLP mimics the three-dimensional conformation of authentic papillomavirus virions.

Accordingly, one aspect of the invention is a method to produce a papillomavirus pseudovirus having a high infectivity to particle ratio, comprising:
a. contacting an intact papillomavirus-like particle (VLP) with a therapeutic nucleic acid molecule to produce a composition; and,
b. incubating the composition under conditions suitable for encapsidation of the therapeutic nucleic acid molecule by the VLP;
   wherein the incubation conditions yield a papilloma pseudovirus having a high infectivity to particle ratio.

In such methods, the papillomavirus VLP can comprise capsid proteins from an alpha-papillomavirus, such as α4, α5, α7v, α8, α9, α4, α10, a beta-papillomavirus, such as β1 or β2 papillomavirus. In such methods, the papillomavirus VLP can comprise a papillomavirus L1 protein, and optionally, a papillomavirus L2 protein. The papillomavirus L1, and optionally L2, protein can each, independently, be at least 85%, at least 90%, at least 95%, at least 97% or 100% identical to an L1 or L2 protein from a papillomavirus selected from the group consisting of HPV1, HPV2, HPV3, HPV4, HPV5, HPV6, HPV7, HPV8, HPV10, HPV11, HPV16, HPV18, HPV26, HPV31, HPV33, HPV34, HPV38, HPV39, HPV40, HPV 41, HPV 42, HPV 43, HPV 44, HPV45, HPV51, HPV52, HPV53, HPV54, HPV55, HPV58, HPV59, HPV68, MmPV1, BPV1, SfPV1, and MusPV1.

In one aspect, the therapeutic nucleic acid molecule is a DNA molecule. The DNA molecule can be a linear DNA molecule having overhanging ends or having blunt ends. The DNA molecule can also be a covalently closed, circular DNA molecule, such as a plasmid. In certain aspects, one or more strands of the covalently closed, circular DNA molecule has been nicked with a nuclease to prevent super-coiling of the DNA molecule. In certain aspects, the DNA molecule encodes a protein selected from a tumor suppressor protein, a pro-apoptotic protein, a protein that causes cell death, a cytokine, a lymphokine, a monokine, a growth factor, an enzyme, an immunomodulatory protein, a cytotoxin, a pro-drug and a single-chain antibody. Encoded cytotoxins may be selected from the group consisting of abrin, Pseudomonas exotoxin A, diphtheria toxin, cholix toxin, cholera toxin, botulinum toxin, pokeweed antiviral protein, and modified toxins thereof.

The therapeutic nucleic acid molecules may also be an RNA molecule. The RNA molecule can be an mRNA molecule, or it can be a functional RNA molecule. In certain aspects, the protein encoded by the mRNA molecule is a tumor suppressor protein, a pro-apoptotic protein, a protein that causes cell death, a cytokine, a lymphokine, a monokine, a growth factor, an enzyme, an immunomodulatory protein, a cytotoxin, a pro-drug or a single-chain antibody. Encoded cytotoxins may be selected from the group consisting of abrin, Pseudomonas exotoxin A, diphtheria toxin, cholix toxin, cholera toxin, botulinum toxin, pokeweed antiviral protein, and modified toxins thereof.

In one aspect, the RNA is a functional RNA. The functional RNA can be selected from the group consisting of siRNA, shRNA, miRNA, cirRNA, snoRNA, snRNA, piRNA, scaRNA, aptamers, and ribozymes.

In such methods, the resulting papilloma pseudovirus particle has an infectivity to particle ratio at least 10%, at least 20%, at least 30%, at least 40%, at least 50%, at last 60%, at least 70%, at least 80%, or at least 90%, of the infectivity to particle ratio of a native papillomavirus of the same type (e.g., HPV16), or a papillomavirus pseudovirus of the same type, produced using methods currently known in the art. In one aspect, the papillomavirus pseudovirus has an infectivity to particle ratio of at least 1×10⁸ i.u./mg L1 protein, at least 5×10⁸ i.u./mg L1 protein, at least 1×10⁹ i.u./mg L1 protein, at least 5×10⁹ i.u./mg L1 protein, at least 1×10¹⁰ i.u./mg L1 protein, at least 5×10¹⁰ i.u./mg L1 protein, or at least 1×10¹¹ i.u./mg L1 protein.

The intact papillomavirus pseudovirus can be contacted with an amount of a therapeutic nucleic acid molecule in the range of at least 50 ng of a therapeutic nucleic acid molecule/ug L1 protein to at least 3 ug of a therapeutic nucleic acid molecule/ug L1 protein. In certain aspects, the amount of the therapeutic nucleic acid molecules can be at least 50 ng of a therapeutic nucleic acid molecule/ug L1 protein, at least 100 ng of a therapeutic nucleic acid molecule/ug L1 protein, at least 150 ng of a therapeutic nucleic acid molecule/ug L1 protein, at least 250 ng of a therapeutic nucleic acid molecule/ug L1 protein, at least 500 ng of a therapeutic nucleic acid molecule/ug L1 protein, at least 750 ng of a therapeutic nucleic acid molecule/ug L1 protein, at least lug of a therapeutic nucleic acid molecule/ug L1 protein, at least 1500 ng of a therapeutic nucleic acid molecule/ug L1 protein, at least 2 ug of a therapeutic nucleic acid molecule/ug L1 protein, at least 2500 ng of a therapeutic nucleic acid molecule/ug L1 protein, or at least 3 ug of a therapeutic nucleic acid molecule/ug L1 protein.

Such methods can further comprise a step in which, following incubation of the composition comprising the intact papillomavirus pseudovirus particle and the therapeutic nucleic acid molecule, the resulting papillomavirus pseudovirus particles are contacted with a nuclease, such as, an exonuclease.

In such methods, the composition comprising the intact papillomavirus VLP and the therapeutic nucleic acid molecule comprises less than about 600 mM NaCl, less than about 300 mM NaCl, less than about 200 mM NaCl, less than about 100 mM NaCl, or less than about 50 mM NaCl. In one aspect, the composition lacks a detectable amount of NaCl.

In such methods, the pH of the composition comprising the intact papillomavirus VLP and the therapeutic nucleic acid molecule, is in the range of about pH 5.2 to about pH 8.2 In one aspect, the pH is less than, or equal to, about pH 7.2, less than, or equal to, about pH 6.5, less than, or equal to, about pH 6.2 less than, or equal to, about pH 5.5, or less than, or equal to, about pH 5.2.

In such methods, the pH of the composition comprising the papillomavirus pseudovirus particle and the therapeutic nucleic acid molecule, can be less than about pH 5.5, and the composition lacks a detectable level of NaCl. In one aspect, the composition has a pH of about pH 5.2, and comprises about 50-150 mM of a buffer, about 0.02% of a surfactant, and at least 50 ng to at least 300 ng of the therapeutic nucleic acid molecule, (e.g., therapeutic nucleic acid molecule) per microgram of L1 protein. The composition can comprise about 100 mM of a buffer such as, citrate buffer, Tris, Trizma, MES, Bis-Tris, ADA, ACES, PIPES, MOPSO, BES, MOPS, TES, HEPES, DIPSO, MOBS, TAPSO, HEPPSO, and POPSO. The surfactant can be a non-ionic surfactant, such as Tween-80 or Triton X-100.

In such methods, the composition comprising the papillomavirus pseudovirus particle and a therapeutic nucleic acid molecule, can have a pH of about pH 5.2, and comprises about 100 mM of citrate buffer, about 0.02% of Tween-80, and at least 50 ng to at least 3000 ng of a therapeutic nucleic acid molecule per microgram of L1 protein.

In one aspect of the invention, the composition comprising the papillomavirus pseudovirus particle and the therapeutic nucleic acid molecule, comprises an amount of NaCl in a range of from about 0 mM NaCl to about 300 mM NaCl, in the range of from about 50 mM to about 300 mM, or in the range of from about 50 mM to about 150 mM. In such methods, the composition can comprise at least about 50 mM NaCl.

In such methods, the pH of the composition comprising the papillomavirus pseudovirus particle and the therapeutic nucleic acid molecule, can be at least pH 6.0. In one aspect, the pH can be in the range of about pH 6.0 to about pH 8.2. In one aspect, the pH of the composition can be in the range of about pH 7.2 to about 8.2.

In such methods, the composition comprising the papillomavirus pseudovirus particle and the therapeutic nucleic acid molecule, has a pH in the range of about pH 7.2 to about pH 8.2, and comprises an amount of NaCl in the range of about 100 mM to about 150 mM. The composition can further comprise a calcium salt, such as, for example, calcium chloride. In one aspect, the composition can comprise a non-ionic surfactant, such as, for example, Tween-80 or Triton X-100. In one aspect, the composition comprising the papillomavirus pseudovirus particle and the therapeutic nucleic acid molecule, can have a pH in the range of about pH 7.2 to about pH 8.2, and comprises about 50-150 mM of a buffer, about 50 mM-150 mM of NaCl, about 0.02% of a surfactant, about 5 mM-20 mM of a calcium salt, and at least 50 ng of a therapeutic nucleic acid molecule per microgram of L1 protein. In one aspect, the composition comprising the papillomavirus pseudovirus particle and a therapeutic nucleic acid molecule, can have a pH of about pH 7.2, and comprises about 100 mM Tris buffer, about 150 mM NaCl, about 0.02% Tween-80, and at least 50 ng of the therapeutic nucleic acid molecule per microgram of L1 protein. In certain aspects, the composition can comprise at least 100 ng of a therapeutic nucleic acid molecule/ug L1 protein, at least 50 ng of a therapeutic nucleic acid molecule/ug L1 protein, at least 100 ng of a therapeutic nucleic acid molecule/ug L1 protein, at least 150 ng of a therapeutic nucleic acid molecule/ug L1 protein, at least 250 ng of a therapeutic nucleic acid molecule/ug L1 protein, at least 500 ng of a therapeutic nucleic acid molecule/ug L1 protein, at least 750 ng of a therapeutic nucleic acid molecule /ug L1 protein, at least 1 ug of a therapeutic nucleic acid molecule /ug L1 protein, at least 1500 ng of a therapeutic nucleic acid molecule/ug L1 protein, at least 2 ug nucleic acid molecules/ug L1 protein, at least 2500 ng of a therapeutic nucleic acid molecule /ug L1 protein, or at least 3 ug of a therapeutic nucleic acid molecule /ug L1 protein.

It is known in the art that VLPs can be disassembled using certain incubation conditions, and then reassembled using a different set of incubation conditions. Methods for disassembling and reassembling papillomavirus VLPs are disclosed in U.S. Pat. No. 6,962,77, and in U.S. Patent Publication No. 2001/0021385, both of which are incorporated herein by reference, in their entirety. Thus, the present disclosure provides vastly improved methods for disassembling and reassembling papillomavirus VLPs to produce papillomavirus pseudovirus particles having much higher infectivity to particle ratios than has been observed or obtained using the methods of the prior art. Such methods of this disclosure can generally be practiced by disassembling a papillomavirus VLP, contacting the disassembled VLP with a therapeutic nucleic acid molecule to form a composition, and incubating the composition under conditions that allow reassembly of the papillomavirus VLP.In these methods, the papillomavirus VLP may be contacted with a therapeutic nucleic acid molecule prior to or during the disassembly process.

Thus, the invention provides methods to produce a papillomavirus pseudovirus having a high infectivity to particle ratio, comprising:
a. contacting a disassembled papillomavirus-like particle (VLP) with a therapeutic nucleic acid molecule to produce a composition; and,
b. incubating the composition under conditions suitable for reassembly of the disassembled, papillomavirus VLP, thereby forming a papillomavirus pseudovirus;
   wherein the reassembly conditions yield a papillomavirus pseudovirus having a high infectivity to particle ratio.

In such methods, the papillomavirus VLP can be disassembled by incubating the papillomavirus VLP under conditions comprising sodium chloride. In certain aspects, papillomavirus VLP can be incubated under conditions comprising NaCl in a range of from about 50 mM to about 200 mM. In certain aspects, the incubation conditions can further comprise a reducing agent, such as, dithiothreitol (DTT). The amount of DTT can be in the range of at least 1 mM to about 5 mM. The amount of DTT can be at least 1 mM, at least 2 mM, at least 3 mM, at least 4 mM, or at least about 5 mM. In certain aspects, the incubation conditions can further comprise a detergent, such as a nonionic detergent, in a range of from about 0.01% to about 0.1%, or from about 0.01% to about 0.05%. Examples of suitable detergents include, but are not limited to Tween-80 and Triton X-100. In such methods, the incubation conditions can comprise between 50 mM and 200 mM NaCl, at least 2 MM DTT, and at least 0.01% Tween-80.

Following disassembly of the papillomavirus VLP, the disassembled VLP can be contacted with the therapeutic nucleic acid molecule to form a mixture, and the conditions of the mixture altered such that the disassembled VLP reassembles, and in the process, encapsidates the therapeutic nucleic acid molecule, thereby forming a papillomavirus pseudovirus. Alteration of the incubation conditions can be achieved by diluting the mixture with a dilution buffer. In such methods, the mixture comprising the disassembled papillomavirus VLPs and the therapeutic nucleic acid molecule is diluted at least 2, at least 4, at least 5, or at least 10-fold, with a dilution buffer. In such methods, the dilution buffer can comprise sodium chloride. The dilution buffer can comprise at least 100 mM NaCl, or at least 100 mM to about 150 mM NaCl. In certain aspects, the dilution buffer can comprise about 150 mM NaCl. The dilution buffer can further comprise a detergent, such as, for example, a non-ionic detergent (e.g., Tween-80, Triton X-100). The concentration of detergent in the dilution buffer can be at least 1%, in the range of at least 0.01% to about 0.04%, or about 0.02%. The dilution buffer can further comprise a calcium salt, such as, for example, calcium chloride. The pH of the dilution buffer can be in the range of about pH 7.2 to about pH 8.2.

In such methods, the dilution buffer can comprise at least 100 mM NaCl, at least 5 mM calcium chloride, and a detergent at a concentration of at least 0.01%. In one aspect, the dilution buffer can comprise about 150 mM NaCl, about 10 mM calcium chloride, and a detergent at a concentration of about 0.02%. In such methods, the pH of the dilution buffer is in the range of about pH 7.2 to about pH 8.2.

Another aspect of this disclosure provides methods to produce a papillomavirus pseudovirus having a high infectivity to particle ratio, comprising:
a. disassembling a papillomavirus VLP by incubating the papillomavirus VLP in solution comprising sodium chloride, a reducing agent, and a detergent;
b. contacting a disassembled papillomavirus-like particle (VLP) with a therapeutic nucleic acid molecule to produce a composition;
c. diluting the composition at least 2-fold using a dilution buffer comprising sodium chloride, calcium chloride, and a detergent; and,
d. incubating the composition under conditions suitable for reassembly of the disassembled, papillomavirus VLP, thereby forming a papillomavirus pseudovirus;
   wherein the reassembly conditions yield a papillomavirus pseudovirus having a high infectivity to particle ratio.

In such methods, the disassembly solution can comprise NaCl in a range of from about 50 mM to about 200 mM. The disassembly solution can comprise reducing agent in the range at least 1 mM to about 5 mM. The reducing agent can be DTT, in an amount of at least 1 mM, at least 2 mM, at least 3 mM, at least 4 mm, or at least about 5 mM. The disassembly solution can comprise a detergent, such as a nonionic detergent (e.g., Tween-80, Triton, X-100), in a range of from about 0.01% to about 0.1%, or from about 0.01% to about 0.05%. Examples of suitable detergents include, but are not limited to Tween-80 and Triton X-100. In certain aspects, the disassembly solution can comprise between 50 mM and 200 mM NaCl, at least 2 mM DTT, and at least 0.01% Tween-80.

In such methods, the composition comprising the disassembled papillomavirus VLPs and the therapeutic nucleic acid molecule can be diluted at least 2, at least 4, at least 5, or at least 10-fold, with the dilution buffer. The dilution buffer can comprise at least 100 mM NaCl, or at least 100 mM to about 150 mM NaCl. In certain aspects, the dilution buffer can comprise about 150 mM NaCl. The dilution buffer can further comprise a detergent, such as, for example, a non-ionic detergent (e.g., Tween-80, Triton X-100), at a concentration of at least 1%, in the range of at least 0.01% to about 0.04%, or about 0.02%. The pH of the dilution buffer can be in the range of about pH 7.2 to about pH 8.2.

In such methods, the dilution buffer can comprise at least 100 mM NaCl, at least 5 mM calcium chloride, and a detergent at a concentration of at least 0.01%. In one aspect, the dilution buffer can comprise about 150 mM NaCl, about 10 mM calcium chloride, and a detergent at a concentration of about 0.02%. In such methods, the pH of the dilution buffer is in the range of about pH 7.2 to about pH 8.2.

In such methods, the papillomavirus VLP can comprise capsid proteins from an alpha-papillomavirus, such as α4, α5, α7v, α8, α9, α4, α10, a beta-papillomavirus, such as (31 or β2 papillomavirus. In such methods, the papillomavirus VLP can comprise a papillomavirus L1 protein, and optionally, a papillomavirus L2 protein. The papillomavirus L1, and optionally L2, protein can each, independently, be at least 85%, at least 90%, at least 95%, at least 97% or 100% identical to an L1 or L2 protein from a papillomavirus selected from the group consisting of HPV1, HPV2, HPV3, HPV4, HPV5, HPV6, HPV7, HPV8, HPV10, HPV11, HPV16, HPV18, HPV26, HPV31, HPV33, HPV34, HPV38, HPV39, HPV40, HPV 41, HPV 42, HPV 43, HPV 44, HPV45, HPV51, HPV52, HPV53, HPV54, HPV55, HPV58, HPV59, HPV68, MmPV1, BPV1, SfPV1, and MusPV1.

In such methods, the therapeutic nucleic acid molecule can be DNA, RNA, modified forms thereof, and combinations thereof, as previously described herein.

In these methods, the therapeutic nucleic acid molecule may be a DNA molecule. The DNA molecule can be a linear DNA molecule having overhanging ends or having blunt ends. The DNA molecule can also be a covalently closed, circular DNA molecule, such as a plasmid. In certain aspects, one or more strands of the covalently closed, circular DNA molecule has been nicked with a nuclease to prevent super-coiling of the DNA molecule. In certain aspects, the DNA molecule encodes a protein selected from a tumor suppressor protein, a pro-apoptotic protein, a protein that causes cell death, a cytokine, a lymphokine, a monokine, a growth factor, an enzyme, an immunomodulatory protein, a cytotoxin, a pro-drug and a single-chain antibody. In one aspect the encoded protein is a cytotoxin. In certain aspects, the encoded protein is a cytotoxin selected from the group consisting of abrin, Pseudomonas exotoxin A, diphtheria toxin, cholix toxin, cholera toxin, botulinum toxin, pokeweed antiviral protein, and modified toxins thereof.

The therapeutic nucleic acid molecule may be an RNA molecule. The RNA molecule can be an mRNA molecule, or it can be a functional RNA molecule. In certain aspects, the protein encoded by the mRNA molecule is a tumor suppressor protein, a pro-apoptotic protein, a protein that causes cell death, a cytokine, a lymphokine, a monokine, a growth factor, an enzyme, an immunomodulatory protein, a cytotoxin, a pro-drug or a single-chain antibody. In one aspect the encoded protein is a cytotoxin. In certain aspects, the encoded protein is a cytotoxin selected from the group consisting of abrin, Pseudomonas exotoxin A, diphtheria toxin, cholix toxin, cholera toxin, botulinum toxin, pokeweed antiviral protein, and modified toxins thereof.

In one aspect, the RNA is a functional RNA. The functional RNA can be selected from the group consisting of siRNA, shRNA, miRNA, cirRNA, snoRNA, snRNA, piRNA, scaRNA, aptamers, and ribozymes.

In such methods, the resulting papillomavirus pseudovirus particle has an infectivity to particle ratio at least 10%, at least 20%, at least 30%, at least 40%, at least 50%, at last 60%, at least 70%, at least 80%, or at least 90%, of the infectivity to particle ratio of a native papillomavirus of the same type (e.g., HPV16), or a papillomavirus pseudovirus of the same type, produced using methods currently known in the art. In one aspect, the papillomavirus pseudovirus has an infectivity to particle ratio of at least 1×10⁸ i.u./mg L1 protein, at least 5×10⁸ i.u./mg L1 protein, at least 1×10⁹ i.u./mg L1 protein, at least 5×10⁹ i.u./mg L1 protein, at least 1×10¹⁰ i.u./mg L1 protein, at least 5×10¹⁰ i.u./mg L1 protein, or at least 1×10¹¹ i.u./mg L1 protein.

The intact papillomavirus pseudovirus can be contacted with an amount of a therapeutic nucleic acid molecule in the range of at least 50 ng of a therapeutic nucleic acid molecule/ug L1 protein to at least 3 ug of a therapeutic nucleic acid molecule/ug L1 protein. In certain aspects, the amount of the therapeutic nucleic acid molecules can be at least 50 ng of a therapeutic nucleic acid molecule/ug L1 protein, at least 100 ng of a therapeutic nucleic acid molecule/ug L1 protein, at least 150 ng of a therapeutic nucleic acid molecule/ug L1 protein, at least 250 ng of a therapeutic nucleic acid molecule/ug L1 protein, at least 500 ng of a therapeutic nucleic acid molecule/ug L1 protein, at least 750 ng of a therapeutic nucleic acid molecule/ug L1 protein, at least 1 ug of a therapeutic nucleic acid molecule/ug L1 protein, at least 1500 ng of a therapeutic nucleic acid molecule/ug L1 protein, at least 2 ug of a therapeutic nucleic acid molecule/ug L1 protein, at least 2500 ng of a therapeutic nucleic acid molecule/ug L1 protein, or at least 3 ug of a therapeutic nucleic acid molecule/ug L1 protein.

The inventors have also discovered that the infectivity to particle ratio can be improved by contacting the papillomavirus pseudovirus particles resulting from the above-described methods, with an oxidizing agent. Thus, the above-disclosed methods may further comprise contacting the papillomavirus pseudovirus particles with an oxidizing agent. In one aspect, the oxidizing agent is oxidized glutathione (GSSG). In one aspect, the papillomaviurs pseudovirus particles are contacted with at least 5 mM oxidized glutathione.

These methods may further comprise contacting the papillomavirus pseudovirus particles with a nuclease, such as, an exonuclease.

One aspect of the invention, is a papillomavirus pseudovirus particle produced according to a method disclosed herein. Such a particle has an infectivity to particle ratio at least 10%, at least 20%, at least 30%, at least 40%, at least 50%, at last 60%, at least 70%, at least 80%, or at least 90%, of the infectivity to particle ratio of a native papillomavirus of the same type (e.g., HPV16), or a papillomavirus pseudovirus of the same type, produced using methods currently known in the art. In one aspect, such a papillomavirus pseudovirus particle has an infectivity to particle ratio of at least 1×10⁸ i.u./mg L1 protein, at least 5×10⁸ i.u./mg L1 protein, at least 1×10⁹ i.u./mg L1 protein, at least 5×10⁹ i.u./mg L1 protein, at least 1×10¹⁰ i.u./mg L1 protein, at least 5×10¹⁰ i.u./mg L1 protein, or at least 1×10¹¹ i.u./mg L1 protein.

Another aspect of this disclosure is a method of delivering a therapeutic nucleic acid molecule into a cell, comprising:
a) obtaining a papillomavirus pseudovirus particle comprising a therapeutic nucleic acid molecule, wherein the papillomavirus pseudovirus particle is produced according to a method disclosed herein; and,
b) contacting a target cell with the papillomavirus pseudovirus particle under conditions that allow entry of the papillomavirus pseudovirus particle into the cell; thereby delivering the therapeutic nucleic acid molecule into the cell.

The target cell can be any cell capable of being infected by a papillomavirus. In one aspect, the target cell is in vitro (e.g., a tissue culture cell). In one aspect, the cell is in vivo (i.e., in a subject). A target cell in a subject can include any desired cell, such as the following cells and cells derived from the following tissues, in humans as well as other mammals, such as primates, horse, sheep, goat, pig, dog, rat, and mouse: Adipocytes, Adenocyte, Adrenal cortex, Amnion, Aorta, Ascites, Astrocyte, Bladder, Bone, Bone marrow, Brain, Breast, Bronchus, Cardiac muscle, Cecum, Cervix, Chorion, Colon, Conjunctiva, Connective tissue, Cornea, Dermis, Duodenum, Endometrium, Endothelium,

Epithelial tissue, Epidermis, Esophagus, Eye, Fascia, Fibroblasts, Foreskin, Gastric, Glial cells, Glioblast, Gonad, Hepatic cells, Histocyte, Ileum, Intestine, small Intestine, Jejunum, Keratinocytes, Kidney, Larynx, Leukocytes, Lipocyte, Liver, Lung, Lymph node, Lymphoblast, Lymphocytes, Macrophages, Mammary alveolar nodule, Mammary gland, Mastocyte, Maxilla, Melanocytes, Monocytes, Mouth, Myelin, Nervous tissue, Neuroblast, Neurons, Neuroglia, Osteoblasts, Osteogenic cells, Ovary, Palate, Pancreas, Papilloma, Peritoneum, Pituicytes, Pharynx, Placenta, Plasma cells, Pleura, Prostate,

Rectum, Salivary gland, Skeletal muscle, Skin, Smooth muscle, Somatic, Spleen, Squamous, Stomach, Submandibular gland, Submaxillary gland, Synoviocytes, Testis, Thymus, Thyroid, Trabeculae, Trachea, Turbinate, Umbilical cord, Ureter, and Uterus.

A related aspect of this disclosure is a method of treating a disease or condition in a subject, comprising:
a) obtaining a papillomavirus pseudovirus particle produced using a method disclosed herein, wherein the papillomavirus pseudovirus particle comprises a therapeutic nucleic acid molecule suitable for treating the disease or condition; and,
b) administering the papillomavirus pseudovirus particle to the subject, thereby treating the disease or condition.

As used herein, treating a condition or disease means causing a clinically significant improvement in one or more clinical signs or symptoms of the condition or disease.

As used herein, the terms subject, individual, patient, and the like, are meant to encompass any mammal capable of being infected by a papillomavirus, with a preferred mammal being a human. The terms individual, subject, and patient by themselves do not denote a particular age, sex, race, and the like. Thus, individuals of any age, whether male or female, are intended to be covered by this disclosure. Likewise, the methods of the invention can be applied to any race of human, including, for example, Caucasian (white), African-American (black), Native American, Native Hawaiian, Hispanic, Latino, Asian, and European. In some embodiments of the invention, such characteristics may be significant.

In such cases, the significant characteristic(s) (e.g., age, sex, race, etc.) will be indicated. Additionally, the terms subject, individual, patient, and the like, encompass both human and non-human animals. Suitable non-human animals to which antisense oligomers of the invention may be administered include, but are not limited to companion animals (i.e. pets), food animals, work animals, or zoo animals. Preferred animals include, but are not limited to, cats, dogs, horses, ferrets and other Mustelids, cattle, sheep, swine, and rodents.

In such methods, the therapeutic nucleic acid molecule can be DNA, RNA, modified forms thereof, and combinations thereof.

In one aspect, the therapeutic nucleic acid molecule is a DNA molecule. The DNA molecule can be a linear DNA molecule having overhanging ends or having blunt ends. The DNA molecule can also be a covalently closed, circular DNA molecule, such as a plasmid. In certain aspects, one or more strands of the covalently closed, circular DNA molecule has been nicked with a nuclease to prevent super-coiling of the DNA molecule. In certain aspects, the DNA molecule encodes a protein selected from a tumor suppressor protein, a pro-apoptotic protein, a protein that causes cell death, a cytokine, a lymphokine, a monokine, a growth factor, an enzyme, an immunomodulatory protein, a cytotoxin, a pro-drug and a single-chain antibody. In one aspect the encoded protein is a cytotoxin. In certain aspects, the encoded protein is a cytotoxin selected from the group consisting of abrin, Pseudomonas exotoxin A, diphtheria toxin, cholix toxin, cholera toxin, botulinum toxin, pokeweed antiviral protein, and modified toxins thereof.

In one aspect, the therapeutic nucleic acid molecule is an RNA molecule. The RNA molecule can be an mRNA molecule, or it can be a functional RNA molecule. In certain aspects, the protein encoded by the mRNA molecule is a tumor suppressor protein, a pro-apoptotic protein, a protein that causes cell death, a cytokine, a lymphokine, a monokine, a growth factor, an enzyme, an immunomodulatory protein, a cytotoxin, a pro-drug or a single-chain antibody. In one aspect the encoded protein is a cytotoxin. In certain aspects, the encoded protein is a cytotoxin selected from the group consisting of abrin, Pseudomonas exotoxin A, diphtheria toxin, cholix toxin, cholera toxin, botulinum toxin, pokeweed antiviral protein, and modified toxins thereof.

In one aspect, the RNA is a functional RNA. The functional RNA can be selected from the group consisting of siRNA, shRNA, miRNA, cirRNA, snoRNA, snRNA, piRNA, scaRNA, aptamers, and ribozymes.

In such methods, papillomavirus pseudovirus particles of the invention can be administered to an individual by any suitable route of administration. Examples of such routes include, but are not limited to, oral and parenteral routes, (e.g., intravenous (IV), subcutaneous, intraperitoneal (IP), and intramuscular), intrathecal, inhalation (e.g., nebulization and inhalation) and transdermal delivery (e.g., topical). The invention also includes any methods effective to deliver a papilloma pseudovirus particle of the invention into the bloodstream of a subject. Parental or intravenous administration, if used, are generally characterized by injection. Injectables can be prepared in conventional forms, either as liquid solutions or suspensions, solid forms suitable for solution or suspension in liquid prior to injection, or as emulsions.

Compositions for administration to a subject can include various amounts of the papillomavirus pseudovirus particle in combination with a pharmaceutically acceptable carrier and, in addition, if desired, may include other medicinal agents, pharmaceutical agents, carriers, adjuvants, diluents, stabilizers, etc. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycollic acids, polymeric amino acids, amino acid copolymers, lipid aggregates, or the like.

Dosages will depend upon the mode of administration, the disease or condition to be treated, and the individual subject's condition, but will be that dosage typical for and used in administration of other pseuodviral vectors and/or VLPs. Often a single dose can be sufficient; however, the dose can be repeated if desirable. Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g., decisions on dosage etc, is within the skills of a medical provider, and typically takes account of the disorder to be treated, the condition of the individual patient, the site of delivery, the method of administration and other factors known to medical practitioners. Examples of the techniques and protocols mentioned above can be found in Remington's Pharmaceutical Sciences, 16th edition, 1980, Osol, A. (ed.).

One example of a condition or disease that can be treated using papillomavirus pseudovirus particles of the invention is cancer or a tumor. Thus, one aspect of the invention is a method of treating a tumor in a subject, comprising:
a) obtaining a papillomavirus pseudovirus particle produced using a method disclosed herein, wherein the papillomavirus pseudovirus particle comprises a therapeutic nucleic acid molecule suitable for treating the tumor; and,
b) administering the papillomavirus pseudovirus particle to the subject, thereby treating the tumor.

As used herein, treating a tumor means inhibiting the growth of, reducing, or killing a tumor. Accordingly, a therapeutic nucleic acid useful for practicing such methods is a nucleic acid molecule capable of inhibiting the growth of a tumor, reducing the size of a tumor, or killing a tumor, or that encodes a protein, or functional RNA molecule, capable of inhibiting the growth of a tumor, reducing the size of a tumor, or killing a tumor.

In such methods, the therapeutic nucleic acid molecule can be DNA, RNA, modified forms thereof, and combinations thereof

In one aspect, the therapeutic nucleic acid molecule is a DNA molecule. The DNA molecule can be a linear DNA molecule having overhanging ends or having blunt ends. The DNA molecule can also be a covalently closed, circular DNA molecule, such as a plasmid. In certain aspects, one or more strands of the covalently closed, circular DNA molecule has been nicked with a nuclease to prevent super-coiling of the DNA molecule. In certain aspects, the DNA molecule encodes a protein selected from a tumor suppressor protein, a pro-apoptotic protein, a protein that causes cell death, a cytokine, a lymphokine, a monokine, a growth factor, an enzyme, an immunomodulatory protein, a cytotoxin, a pro-drug and a single-chain antibody. In one aspect the encoded protein is a cytotoxin. In certain aspects, the encoded protein is a cytotoxin selected from the group consisting of abrin, Pseudomonas exotoxin A, diphtheria toxin, cholix toxin, cholera toxin, botulinum toxin, pokeweed antiviral protein, and modified toxins thereof.

In one aspect, the therapeutic nucleic acid molecule is an RNA molecule. The RNA molecule can be an mRNA molecule, or it can be a functional RNA molecule. In certain aspects, the protein encoded by the mRNA molecule is a tumor suppressor protein, a pro-apoptotic protein, a protein that causes cell death, a cytokine, a lymphokine, a monokine, a growth factor, an enzyme, an immunomodulatory protein, a cytotoxin, a pro-drug or a single-chain antibody. In one aspect the encoded protein is a cytotoxin. In certain aspects, the encoded protein is a cytotoxin selected from the group consisting of abrin, Pseudomonas exotoxin A, diphtheria toxin, cholix toxin, cholera toxin, botulinum toxin, pokeweed antiviral protein, and modified toxins thereof.

In one aspect, the RNA is a functional RNA. The functional RNA can be selected from the group consisting of siRNA, shRNA, miRNA, cirRNA, snoRNA, snRNA, piRNA, scaRNA, aptamers, and ribozymes.

In these methods of treating a tumor or cancer, the papillomavirus pseudovirus particle may be administered in conjunction with a cytotoxic agent that inhibits or prevents the function of cells and/or causes destruction of cells. The term is intended to include radioactive isotopes (e.g., At211, 1131, 1125, Y90, Re186, Re188, Sm153, Bi212, P32 and radioactive isotopes of Lu), immunotherapeutic agents, chemotherapeutic agents, enzymes and fragments thereof such as nucleolytic enzymes, antibiotics, and toxins such as small molecule toxins or enzymatically active toxins of bacterial, fungal, plant or animal origin, including fragments and/or variants thereof, and various antitumor or anticancer agents.

Chemotherapeutic agents are chemical compounds useful in the treatment of cancer. Examples of chemotherapeutic agents that may be administered in conjunction with the papilloma pseudovirus particles created by the methods of this disclosure include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti- adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin

A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATINTM) combined with 5-FU and leucovovin.

Also included in this disclosure are kits useful for practicing the disclosed methods. A kit may include nucleic acid molecules, proteins or VLPs necessary for practicing the invention. These kits may also contain at least some of the reagents required to produce such nucleic acid molecules, proteins and/or VLPs. Such reagents may include, but are not limited to, isolated nucleic acid molecules, such as expression vectors, primers, sets of primers, or an array of primers.

The kit may also comprise instructions for using the kit, and various reagents, such as buffers, necessary to practice the methods of the invention. These reagents or buffers may be useful for producing or administering papilloma pseudovirus particles of the invention to a cell or an individual. The kit may also comprise any material necessary to practice the methods of the invention, such as syringes, tubes, swabs, and the like.

### EXAMPLES

### Materials and Methods Used in the Experiments Described in this Experimental Section

Cell lines, antibodies and reagents: 293H (Invitrogen), HeLa (ATCC) and 293TT (Christopher B. Buck, NCI) cells were grown in Dulbecco modified Eagle medium

(DMEM) supplemented with 10% fetal bovine serum (DMEM-10). Decanoyl-RVKR-chloromethilketone (dec-RVKR-cmk), compound XXI (S,S)-2-[2-(3,5-Difluorophenyl)-acetylamino]-N-(1-methyl-2-oxo-5-phenyl-2,3-dihydro-1H-benzo[e][1,4]diazepin-3-yl)-propionamide), Cyclosporin A were from Calbiochem (no. 344930 no. 565790 and no. 239835 respectively). NH₄Cl (A0171), Heparin (H4784), benzonase (E1014) and L-Glutathione oxidized (G4376) were from Sigma. BAL-31 nuclease (M0213) was from New England Biolabs. Plasmid Safe DNase (E3110K) was from Epicentre. H16.V5 was a kind gift from Neil Christensen (Christensen, N. D., et al., 1996; Virology 223, 174-84).

VLP and PsV production: L1-L2 VLPs were produced according to the standard protocol described by Buck and Thompson (Buck and Thompson, 2007; Production of papillomavirus-based gene transfer vectors. Current protocols in cell biology. Editorial board, Juan S Bonifacino, et. al., Chapter 26, Unit 26 21) with the substitution of 293H cells for production. Briefly, 293H cells were transfected with an L1-L2 bicistronic expression plasmid. 48 h post transfection cells were harvested and virus matured for 24 h at 37° C. in Dulbecco's phosphate-buffered saline with calcium and magnesium supplemented with 9.5 mM MgCl₂, 0.25% Brij58 (Sigma), 0.1% Plasmid Safe DNase, 0.1% Benzonase and 25 mM ammonium sulfate pH 9.0. The cell lysate was then chilled and NaCl concentration was adjusted to 0.8M. Cell lysate was then clarified by centrifugation for 20 min at 20 xg. VLPs were purified from the clarified lysate on a 27%/33%/39% Optiprep gradient.

PsV production was performed as described for VLPs but 293TT cells were utilized for production. A GFP-only plasmid or a firefly luciferase and GFP—(Luc/GFP) double expression plasmid was co-transfected with the L1L2 expression plasmid. Maturation and purification was done as described for VLPs.

The following plasmids were used for L1 and L2 expression: p2sheLL, p5sheLL, p6sheLLr, p16sheLL, p18sheLL, p31sheLL, p35sheLL, p45sheLL, p52sheLL, p58sheLL for HPV2, 5, 6, 16, 18, 31, 35, 45, 52 and 58 respectively. For the animal papillomavirus pSheLL, pCRPVsheLL, pMusheLL, pRhSheLL were used to produce BPV1, MmPV1, MusPV1 and SfPV1 respectively. pfwB was used as GFP-reporter plasmid (GFP plasmid), and pCLucf for firefly luciferase and GFP-reporter plasmid (Luc/GFP plasmid). All plasmids are described on the World Wide Web site of the National Cancer Institutes,

Center for Cancer Research, more specifically, on the Lab of Cellular Oncology Technical Files plasmid page. For HPV8, HPV26, HPV38, HPV39, HPV40, HPV59, HPV68 production, we used the plasmids pVITRO-HPV8 L1L2, pVITRO-HPV8 L1L2, pVITRO-HPV26 L1L2, pVITRO-HPV38 L1L2, pVITRO-HPV39 L1L2, pVITRO-HPV40 L1L2, pVITRO-HPV59 L1L2 and pVITRO-HPV68 L1L2 respectively. All pVITRO constructs were a kind gift of Richard Roden, Department of Pathology, The Johns Hopkins University and have been described by Kwak and colleagues (PloS One 9, e97232).

Plasmid production and linearization: All plasmids were produced in competent Escherichia coli DH5aα (BIO-85026, Bioline) and purified using QIAGEN Plasmid Plus Midi Kit (Qiagen). pfwB was linearized using Sbfl (New England Biolabs) and pCLucf with XmnI (New England Biolabs). pCLucf was digested with EcoRI (New England Biolabs) for the “linear split” virus. Digestion was confirmed by agarose gel electrophoresis and enzymes were heat inactivated according to the manufacturer instructions before use. For blunt DNA, pfwB was digested with Pm1I and SbfI. The digested fragment containing GFP was gel purified before usage.

mRNA production: mRNA was produced using the kit HiScribe™ T7 ARCA mRNA Kit with tailing (New England Biolabs®, E2060S) according to the manufacturer instructions. The kit allows the production of mRNA containing a 7-methyl guanosine cap structure at the 5′ end and a Poly(A) tail at the 3′ end of the mRNA. As templates for mRNA, pClneo-GFP and pCLucF were used for eGFP and Firefly Luciferase, respectively. Both plasmids were digested with Notl-HF (New England Biolabs®, R3189) before mRNA production. For the toxin mRNA, the plasmids pPE64 and pPE64Δ553 were used. PE plasmids were linearized with EcoRI-HF (New England Biolabs®, R3101). Before mRNA production, linearized plasmids were purified using QIAquick® Purification Kit (Qiagen®) as directed by the manufacturer instructions. The sequences of pCIneo-EGFP and pCLUcF can be found on the plasmids World Wide Web page, in the Laboratory of Cellular Oncology Technical Files, at the Center for Cancer Research, National Cancer Center. The PE64 plasmids have been described previously (FitzGerald et al., 1998; Hertle et al., 2001)

Reassembly and Nuclear extract preparation: Preparation of nuclear extract from 293H cells and the general reassembly reactions were performed as described previously with minor changes (Cerqueira et al., 2015; supra). All PV VLPs except BPV were disassembled in buffer containing 100 mM NaCl, 20 mM Tris, pH 8.2, 2 mM dithiothreitol (DTT), and 0.01% Tween 80 for 3 h at 37° C. BPV was disassembled in buffer containing: 50 mM NaCl, 20 mM Tris, pH 8.2, 2 mM dithiothreitol (DTT), and 0.01% Tween 80 for 3 h at 37° C.

For packaging DNA, 1 microgram of disassembled or intact (i.e. not disassembled) VLPs were incubated for 20 h at 37° C. in buffer containing 100 mM Tris, pH 7.2, 0.02% Tween 80, 10 mM CaC12, 150 ng of the indicated DNA type (circular, linear, or blunt) in the presence or absence of nuclear extract from 293H cells. Samples were then nuclease treated for 6 h at 37° C. with 0.1% benzonase, 0.1% BAL-31, and 10 mM MgCl₂. When different pH and NaCl concentrations were tested, the reassembly mixture was changed to 100 mM citrate buffer pH 5.2 and 6.2 for pH 5.2 and 6.2 or 100mM Tris pH 7.2 or Tris pH 8.2 for pH 7.2 and 8.2 respectively. CaC12 was omitted from the buffer for the pH 5.2 and 6.2 reactions due to the formation of a calcium precipitate under these conditions. For DNA titration within the assembly reactions, the indicated amounts of DNA were added to the reassembly mix.

For packaging mRNA, disassembled or intact VLPs were incubated for 20 h at 37° C. in buffer containing 100 mM Tris, pH 7.2, 0.02% Tween 80, 10 mM CaCl and different mRNA amounts. Samples were then nuclease treated for 3 h at 37° C. with 0.1% benzonase, 0.1% BAL-31 for DNA or with 0.2% RNase cocktail for RNA. The eGFP mRNA used for initial experiment (Table 1) was from TriLink® Biotechnologies. All further experiments were performed with mRNA produced “in house” as described in the mRNA production section above.

Production of high titer stocks: For HPV16, and other intact particles stocks, particles were diluted in 100 mM Citrate Buffer pH 5.2, 0.02 1% Tween 80 and 450 ng of linear Luc/GFP expression vector (pCLucf) per microgram of L1 protein. Samples were incubated for 30 h at 37° C. A total of 1 μg L1 per 5-11 μl of reaction was used. Samples were incubated for a further 15 h with 5 mM GSSG. Particles were treated for 3 h at 37° C. with 0.2% BAL-31 and 0.2% benzonase for 3 h at 37° C. in buffer containing 10 mM MgCl2 and 0.5 M NaCl. Samples were partially purified and concentrated by cushioning the virus for 1 h at 50,000 rpm on a SW55Ti rotor on a 39% OPTIPREP™ cushion. The virus-containing fraction (immediately above the cushion) was collected and used for further characterization. For HPV45, and other disassembled particles, VLPs were first disassembled for 6h at 37° C. in 200 mM NaCl, 20 mM Tris pH 8.2, 2 mM DTT, and 0.01% Tween80. Once disassembled, particles were reassembled in buffer containing 100 mM Tris pH 7.2, 150 mM NaCl, 10 mM CaCl2, 0.02% Tween80 and 450 ng/us linear Luc/GFP expression vector (pCLuct). For the reassembly, the disassembly mixture was diluted 5× with the reassembly buffer. Samples were incubated for 30 h at 37° C. and then incubated for further 15 h with 5 mM GSSG. Nuclease treatment and purification and concentration was perfoiined as for HPV16.

For particles packaging mRNA, production of high titers stocks was performed similar to that described previously for DNA packaging(Cerqueira et al., 2017). VLPs were disassembled in 200 mM NaCl, 20 mM Tris pH 8.2, 2 mM DTT and 0.01% Tween80for 6 h at 37° C. The disassembled particles were then diluted 5× and reassembled in buffer containing 100 mM Tris pH 7.2, 150 mM NaCl, OmM CaCl2, 0.02% Tween80 and 60 molecules of RNA per capsid. Reassembly reaction was incubated 30 h at 37° C. After this time 5 mM GSSG was added and samples incubated for further 15 h. Particles were treated for 3 h at 37° C. with 0.2% RNase cocktail in buffer containing 10 mM MgCl2 and 0.5 M NaCl. PsVs resulting from the reaction were purified and concentrated by cushioning on a 1 ml 39% Optiprep by centrifugation for 1 h at 50,000 rpm in a SW55Ti rotor.

L1 quantification: Virus samples and BSA standards were analyzed by SDS-PAGE. Gels were either stained with Coomassie (SIMPLYBLUE™ SafeStain, LC6060 ThermoFisher Scientific) or with Sypro Ruby for concentrations lower than 0.10 mg/ml (S12000, ThermoFisher Scientific). Band intensities were determined with the ImageJ 1.49 v software and L1 concentration inferred from the BSA calibration curve.

Virus titration: Titration was based on GFP-expression in 293TT or HeLa cells. About 24 h before infection, 293TT cells in DMEM-10 were pre-plated in a 24-well plate at 1×10⁵ cells in 0.5 ml per well. Cells were infected with 10-fold serial dilutions of the virus stock beginning with 1 μl. Infection, corresponding to GFP expression, was analyzed at 50 hours post infection by flow cytometry.

qPCR: Reporter plasmid copy numbers were determined by qPCR using Taqman™ Assay (Thermo Fisher Scientific™). Encapsidated DNA was extracted from the standard or defined virus preparation. 10 μl virus preparations were incubated at 50° C. for 15 min with 90 μl of extraction buffer (20 mM Tris pH 8.0, 20 mM DTT, 20 mM EDTA, 2.0% SDS, 0.2% Proteinase K). DNA was purified using QIAquick® Purification Kit (Qiagen®) as directed by the manufacturer instructions. qPCR was performed according the manufacturer instructions using the following primers and probe: forward primer 5′-CGGCATCAAGGTGAACTTCA-3′ (SEQ ID NO:7); reverse primer 5′-ACCATGTGATCGCGCTTCTC-3 (SEQ ID NO:8)' and probe: 5′-CCACTACCAGCAGAACA-3′ (SEQ ID NO:9) with 6FAM as dye and MGB-NFQ as quencher using the Applied BiosystemsR 7900 HT Fast Real-Time PCR system. The primers and probe were designed to amplify the GFP gene. To determine the copy number were used known amounts of pCLucf plasmids as standards. The standards ranged from 10⁹ to 10⁵ copies.

### qRT-PCR

mRNA copy numbers were determined by qRT-PCR. Encapsidated mRNA was extracted from the standard or defined virus preparation. 10 μl virus preparations were incubated at 50° C. for 15 min with 90 μl of extraction buffer (20 mM Tris pH 8.0, 20 mM DTT, 20 mM EDTA, 2.0% SDS, 0.2% Proteinase K). mRNA from virus was purified using MagMAX™ Viral RNA Isolation Kit (Life technologies, AM1939) per the manufacturer instructions. qRT-PCR was performed using TaqMan® RNA-to-CT™ 1-Step Kit (Life Technologies, 4392938). The following probes and primers were used for PE: forward (fwd) primer: 5′-AAAGCGCTGGAGCGAATG-3′ (SEQ ID NO:1), reverse (rev) primer: 5′-GATGACCGTGGGTTTGATGTC-3′ (SEQ ID NO:2), Probe: 5′-AAGGTGTTGTGCCTGCT-3′ (SEQ ID NO:3); for Luciferase: fwd primer: 5′-CTGAACAGCATGGGCATCAG-3′ (SEQ ID NO:4), rev primer: 5′-GGGTGGCAAATGGGAAGTC-3′ (SEQ ID NO:5), Probe: 5′-CAGCCCACCGTCGTA-3′; for eGFP (SEQ ID NO:6): fwd primer: 5′-CGGCATCAAGGTGAACTTCA-3′ (SEQ ID NO:7), rev primer: 5′-ACCATGTGATCGCGCTTCTC-3′ (SEQ ID NO:8), Probe: 5′-CCACTACCAGCAGAACA-3′ (SEQ ID NO:9). For comparison, known amounts of mRNA standards, ranged from 10¹⁰ to 10⁶ copies were used.

### Cell Viability

Cell viability was measured using TACS® XTT Cell Proliferation Assay (Cat# 4891-025-K) from Trevigen® according to the manufacturer instructions. Viability percentage was determined taking as 100% untreated cells.

### Neutralization Assays and Inhibition of Infection by Entry Inhibitors

About 24 h prior to infection 4.5×103/well 293TT or HeLa cells were seeded in 96 well plates. For neutralization of infection with heparin or neutralizing antibodies we followed the neutralization protocol described previously (Buck et al., 2005; Methods in Molecular Medicine, 119:445-62; Pastrana et al., 2004; Virology 321:205-16). Briefly, PsVs were incubated with 10-fold dilutions of heparin or a neutralizing antibody (H16.V5 for HPV16 or a polyclonal serum for HPV45) and incubated for lh on ice. After this time, PsV mixtures were added to the preplaced cells. Infection, corresponding to GFP-expression, was scored 72 hours post infection by flow cytometry. When indicated, cells were preincubated for 30 min in DMEM-10 containing inhibitors at the following concentrations: 10 μM dec-RVKR-cmk; 20 mM NH4Cl (10 mM HEPES); 300 nM compound XXI; 10 μM Cyclosporin A (CsA), or left untreated. Cells were infected with 4×10⁴ i.u. in the presence of the indicated inhibitors. Infection was scored 72 h p.i. by flow cytometry. Inhibitors were kept during the complete infection course.

Electron Microscopy: Samples were negatively stained with 0.5% uranyl acetate for 1 s after adsorption to carbon-coated copper grids. Examination of the samples was performed with an FEI Tecnai T12 transmission electron microscope.

Mouse vaginal tract infection: Nine to twelve-week-old female BALB/c were housed in the NCI animal care Facilities according to the National Cancer Institute's Animal Care and Use Committee. Infection was performed as previously described (Roberts et al., 2007; Nature Medicine 13:857-61). Shortly, mice were treated with 3 mg of Depo-Provera (Pfizer) diluted in PBS, 4-5 days before pseudovirus infection. Five hours before infection, 50 μl of 4% Nonoxynol-9 (N-9, N1217 Spectrum) in 4% carboxymethylcellulose (CMC, C4888 Sigma) were instilled intravaginally. After this time 20 μl of the indicated 1×10⁷ infectious units of the indicated PsVs were instilled in the vaginal tract. Virus instillation was done in 2% CMC. Mice were imaged at different times following infection by intravaginal instillation of 20 μl of firefly luciferase substrate (15 mg/ml stock; PerkinElmer). Mice luminescent images were acquired using a 30-sec exposure at medium binning and f/1 on a IVIS 100. Bioluminescence was measured in regions of interest (ROI) around the mouse vagina.

### Vaccinia Virus Infection

About 6 h prior to infection 2×10⁴ HeLa cells were seeded on 96-wells. After the cells had attached, the media was exchanged with fresh media containing the indicated amounts and cells infected with Vaccinia-Virus-GFP (kind gift from Heather Hickman, NIAID, NIH). About 16 h p.i. cells were trypsinized, fixed with 4% paraformaldehyde and GFP expression analyzed by flow cytometry. Final infection for untreated samples was about 50-60%.

### Example 1: Papillomavirus Capsid Proteins Package DNA Independent of Nuclear Components

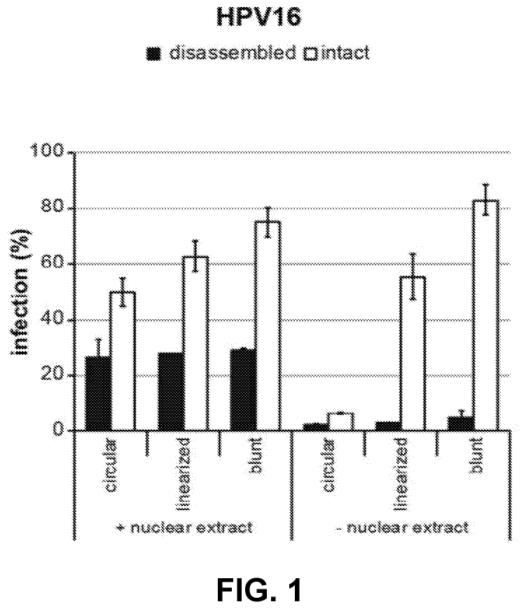

Beginning with the inventors' previously described HPV16 PsV production reaction (Cerqueira et al., 2015; Journal of Virology 90:1096-107), linearized and supercoiled circular GFP-reporter plasmid (GIP plasmid) were compared as the packaging substrates. For the packaging reaction, HPV16 L1/L2 VLPs were disassembled using low salt and DTT, or left intact and the resulting capsid proteins were mixed with the supercoiled circular or linearized DNA in the presence or absence of nuclear extract. The plasmid was linearized by cutting it with a restriction enzyme at a single site that did not disrupt the GFP gene or promoter, followed by heat inactivation of the enzyme. After reassembly, all unpackaged plasmid DNA was digested for 6 h with nucleases. Successful packaging of the plasmid was functionally evaluated by quantifying infection of HeLa cells as determined by the percent of green fluorescing cells using flow cytometry. For the supercoiled circular DNA, results were consistent with those previously reported (Cerqueira et al., 2015, supra) (FIG. 1). Packaging of the circular plasmid into infectious PsVs required the presence of nuclear extract for both disassembled and intact capsid proteins (FIG. 1). More infectious PsVs were generated when intact particles were used. For the linearized plasmid, superior PsV formation was observed with both disassembled and intact particles in the presence of nuclear extract. Surprisingly, there was also substantial PsV assembly using the linear plasmid and intact particles even in the absence of nuclear extract (FIG. 1). Because the plasmid had been linearized using a restriction enzyme that produces overlapping ends, it possibly could have re-circularized during the reaction. Therefore, the assembly reactions were repeated using a linearized plasmid with blunt ends. Specifically, a small piece of the plasmid outside of the GFP expression cassette was removed, to ensure the plasmid would not re-circularize. The infection results using this “blunt” plasmid were comparable with those of the linearized plasmid under all combinations of assembly state and nuclear extract addition. (FIG. 1). A relaxed plasmid, in which single stranded nicks were introduced, was also tested. The results were similar to those using the supercoiled circular plasmid, with infectious PsV generated only in the presence of nuclear extract (data not shown).

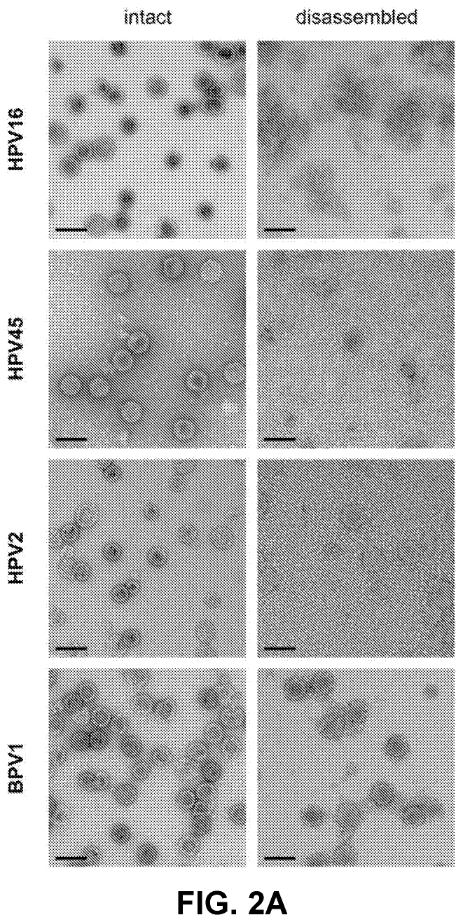

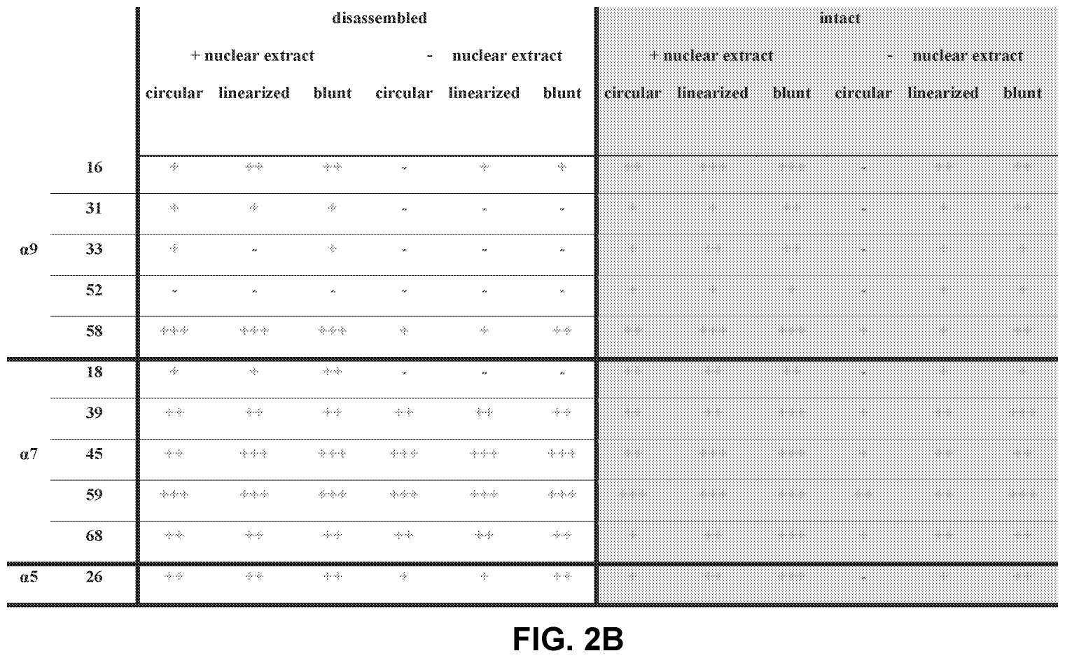

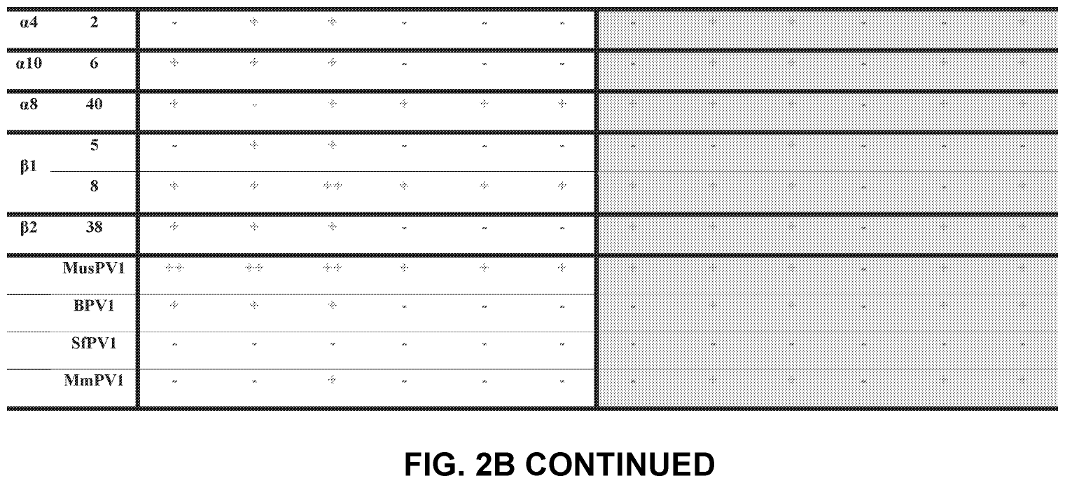

The inventors next investigated the generation of infectious PsV in the cell free assembly methods of this disclosure for 20 additional PV types. The PsV production reactions were preformed either with disassembled or intact particles using circular, linearized or blunt DNA in the presence or absence of nuclear extract for HPV types 16, 31, 33, 52, and 58 (α9 types), HPV types 18, 39, 45, 59, and 68 (α7 type), HPV2 (α4 type), HPV26 (α5 types), HPV 6 (α10 type), HPV types 5 and 8 (β1 type), HPV38 (β2 types) and also the animal types BPV1, MusPV1, MmPV (formerly MTV') and SfPV1 (formerly CRPV1). Electron-photomicrographs confirmed that all PV types disassembled using the conditions established for HPV16 (FIG. 2A and data not shown). The only exception was BPV1, which, even in 50 mM NaCl and 2 mM DTT, resisted disassembly, and exhibited, at best, somewhat expanded capsids after treatment (FIG. 2A). BPV1 has more L1 disulfide bonds than most other PV capsids, which probably explains the higher resistance to disassembly. Several additional disassembly conditions were tested for BPV1, including higher DTT concentrations and longer disassembly incubations (up to 16 h), but none of the tested conditions led to complete disassembly. The inventors nonetheless analyzed. BPVI packaging under standard destabilization conditions, and the BPV capsids generated under these conditions are, for simplicity, referred to as “disassembled” in this disclosure and the accompanying Figures.

To standardize the results of the initial survey of cell-free in vitro PsV production across PV types, the inventors infected HeLa cells with an equivalent amount of total L1 protein for all types, and defined intervals for levels of infectivity (FIG. 2B).

For the α9 types, generating infectious PsV with circular DNA using either disassembled or intact particles required the presence of nuclear extract. The exception was HPV58, which could generate low amounts of PsV with circular DNA in the absence of nuclear extract. Linearized or blunt DNAs were generally better substrates for packaging into intact particles than were circular DNAs, as observed for HPV16. The packaging of linearized DNA into intact particles was nuclear extract-independent for all tested α9 types. Another exception among the α9 group was that HPV58 generated similar infectious PsV titers with disassembled and intact VLPs, while for other types infectivity was better with intact particles.

The PsV production pattern for α7 types differed notably from that of the α9 types. Infection was very high for most representatives, except for HPV18. HPV18 PsV production had a pattern very similar to the one described for HPV16. All other α7 types tested, HV39, 45, 59, and 68, appeared to package all forms of the pseudogenome to high degrees when disassembled. Disassembled VLPs generated more PsV than intact particles, in contrast to most of the α9 types tested. Also, generating PsV from circular DNA and disassembled particles of the latter α7 types was not dependent of the nuclear extract. High infection rates were observed with previously disassembled particles regardless of the presence of nuclear extract. For the intact particles, although packaging seemed generally to be better with nuclear extract, there was also substantial packaging in the absence of nuclear extract. In general, linear DNA seemed to be a better substrate than circular DNA for generating α7 type PsVs.

VLPs of HPV26, an α5 type, could also efficiently generate PsVs. Disassembled particles packaged all of the pseudogenome forms tested in the presence or absence of nuclear extract, although circular DNA packaging was better in the presence of nuclear extract. With intact particles, packaging of circular or linear DNA occurred in the presence of nuclear extract. In the absence of nuclear extract only linear DNA could be packaged with blunt DNA being slightly superior to linearized.

The VLPs of HPV2, an α4 type, HPV6, an α10 type, and HPV40, an α8 type, were inefficient at generating PsV in the in vitro reactions. Low levels of infection were observed under a limited number of reaction conditions, for instance when linear or blunt DNA was added to dissembled or not dissassembled VLPs in the presence of nuclear extract.

Three cutaneous beta HPVs were also examined. For the two β1s, production was low in general, although HPV8 produced more PsV than HPV5 under most reaction conditions, perhaps reflecting the low yields of HPV5 PsV in a standard intracellular production system. For HPVS the inventors could observe very low infection only for linearized or blunt DNA in the presence of nuclear extract. Generation of infectious PsV by HPV38 (a β2 type) was also low under the best of conditions.

For the animal types, infectivity was generally very low or absent under the various reactions condition (FIG. 2B). The notable exception was that disassembled MusPV1 VLPs were reasonably efficient at generating PsV from all three forms of the psuedogenome when mixed with nuclear extract. BPV1 infectivity was very low and only observed under a subset of conditions. Because MmPV1 and SfPV1 generated few or no infectious PsV under any of the experimental condition when assayed on HeLa cells, the inventors also tried infecting 293TT cells. There was no infection with MmPV1 in either cell line, and, for SfPV1, only low infection was observed with linear or blunt DNA in intact particles. The results for MmPV1 were not surprising because this virus is also deficient in generated PsVs in the standard cell culture production system. In contrast, SfPV1 generates substantial titers of PsV in the cell culture system.

Taken together, these results show that PV can package circular and linear DNA under cell free conditions. Unexpectedly, many virus types could generate infectious PsV in the absence of nuclear components, but the best pseudogenome substrate for packaging varied among the PV type. Members of the α7 clade were exceptionally proficient at generating infectious pseudovirions under these cell-free reaction conditions.

### Example 2: Optimizing Cell-Free Production of Papillomavirus Vectors

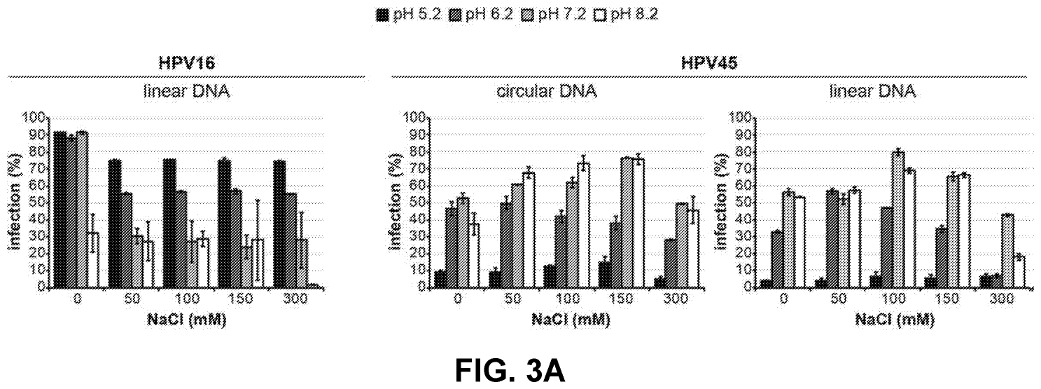

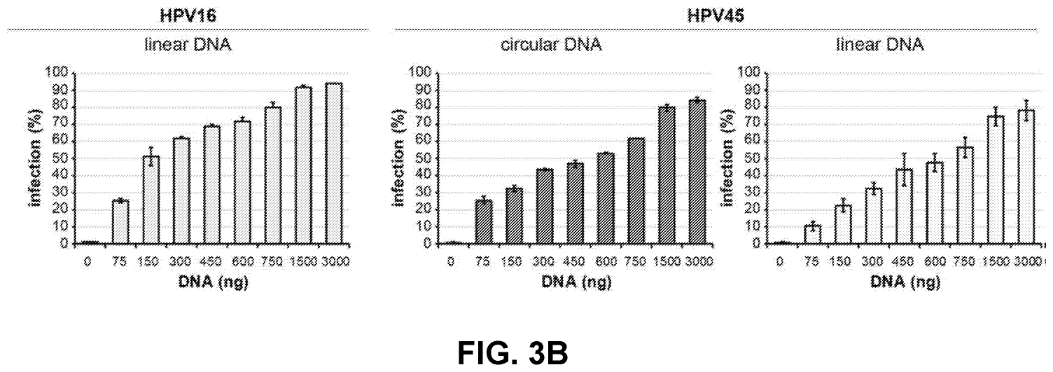

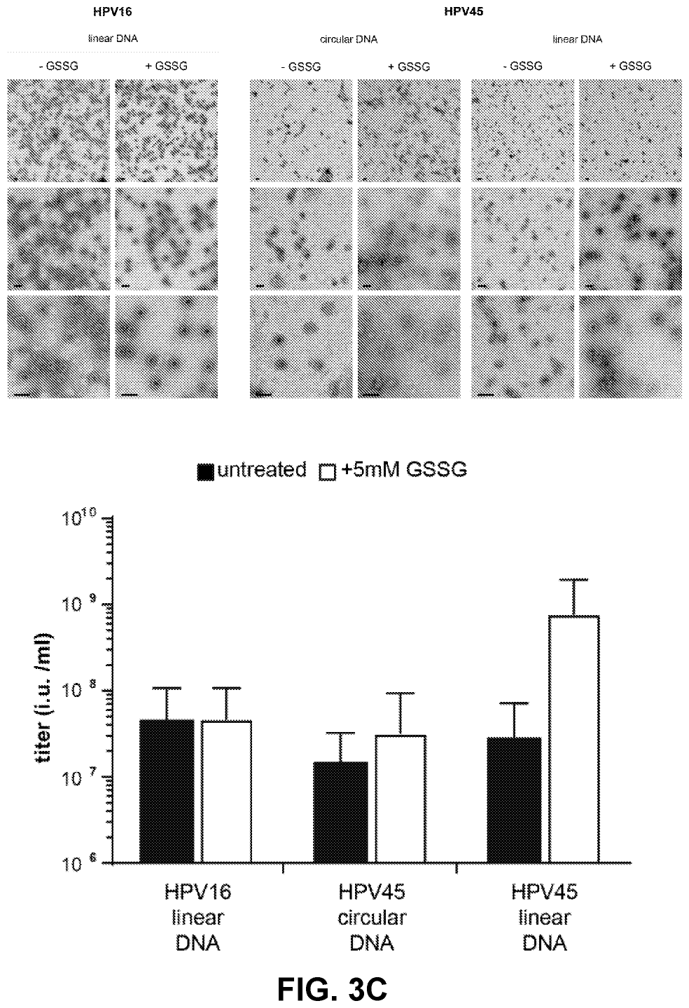

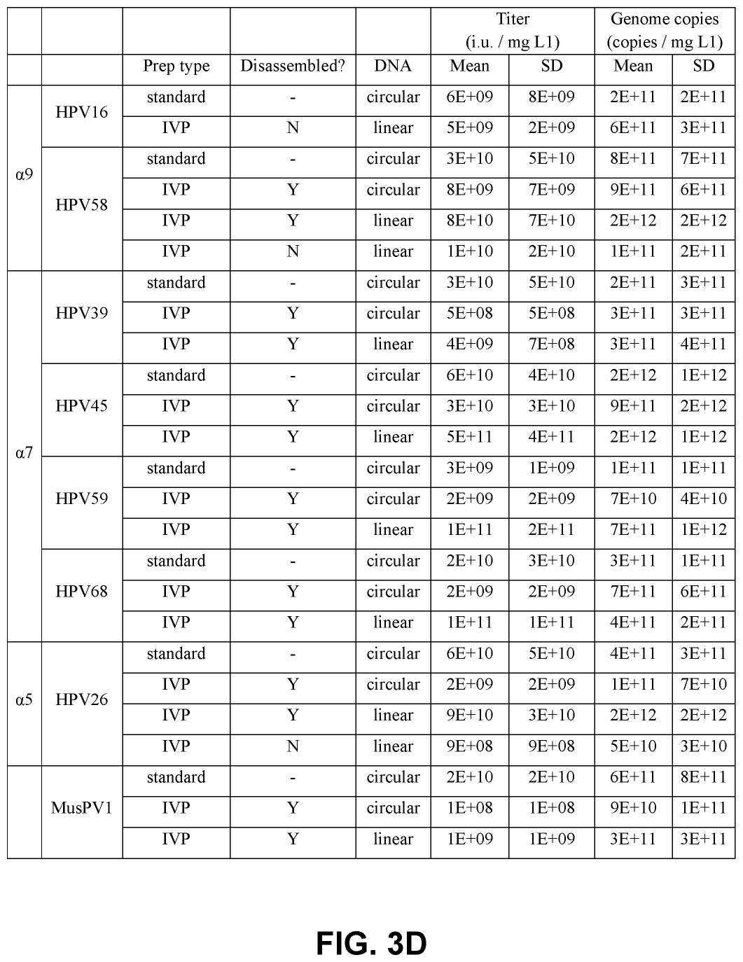

The results presented in Example 1 showed that the capsid proteins from 18 of the 21 PVs tested could generate at least some infectious PsVs using linear DNA as the pseudogenome substrate in the absence of a nuclear extract, and 9 of 21 types could also package circular DNA in the absence of nuclear extract. To produce the simplest possible cell free production scheme possible, the inventors therefore focused on trying to optimize the in vitro generation of PsV without the extract, herein designated as “defined” assembly reactions. HPV16 was chosen as a prototype of a virus that preferentially packages linear DNA into intact particles and HPV45 as an example of virus that efficiently packages both circular and linear DNA into disassembled particles. These types were also chosen because they produce high numbers of highly-concentrated HPV45 VLPs for use as a starting material in the reactions. Different pH and NaCl concentrations were tested in the reassembly reactions. Intact HPV16 VLPs and disassembled HPV45 VLPs were mixed with linear or circular DNA in pH 5.2, 6.2, 7.2, or 8.2 buffer in combination with NaCl concentrations ranging from 0 to 300 mM NaCl (FIG. 3). HeLa infection was used as a measure of PsV production. During the infectivity assays, the NV would be added to the cells in different salt concentrations and pHs, and these alterations might affect infectivity. Therefore, the cells were also infected in the presence of each of the salt and pH combination with standard PsV derived via intracellular production. None of the tested. conditions affected infectivity of control PsVs, indicating that any effects on infectivity were related to the efficiency of PsV production. Additionally, none of the combination of salt and pH conditions affected nuclease treatment, indicating that differences in infectivity were not the result of inhibition of DNA digestion leading unspecific plasmid transfection. At salt concentrations of 600 mM NaCl., PsV production was greatly reduced, and at higher concentrations there is no infection. PsV production for HPV16 was similar at NaCl concentrations from 50 to 300 mM but increased with decreasing pH. When no salt was added to the buffer, production was notably higher, and essentially the same at PH 5.2, 6.2, and 7.2 (FIG. 3A). Infectivity was reduced at pH 8.2 when compared to all other pHs. The trend toward increased PsV production at lower pH and NaCl concentrate suggests that ionic interactions between the psueodogenome and the capsid interior may be important in the packaging reaction. It is important to note that VLPs are produced in the presence of 500 mM salt and diluted for the reassembly reaction, and so the amount of NaCl added to the reaction buffer can vary depending upon the L1 concentration of the VLP preparation. As our standard conditions for HPV16 PsV production, we decided to use pH 5.2 with no added salt in the reaction buffer, since these conditions would assure good packaging even if substantial amounts of salt were added when more dilute VL:Ps preparation were used in the reactions.

For HPV45, there was almost no infectious PsV generated at pH 5.2 when either circular or linear DNA was used, perhaps reflecting an inhibition of capsomer assembly at low pH (FIG. 3A). At pH 6.2, substantial infectivity was observed, but infectivity was generally better at pH 7.2 and 8.2 for both conformations of plasmids tested (FIGS. 3A and 3B). At pH 7.2 and 8.2, PsV production tended to peak at 100-150 mM NaCl.

Concentrations of 600 mM NaCl or higher prevented PsV assembly. pH 7.2 and 150 mM NaCl were chosen for both circular and linear pseudogenomes, as these conditions were close to physiological conditions, and also gave high infectivity.

The impact of pseudogenome concentration on PsV production was also tested. In previous experiments, 50 ng DNA/1 μg L1 protein was routinely used, corresponding to an approximated 1:1 ratio of assembled capsids to DNA molecules. A range from 75 ng to 3,000 ng of linearized GFP-plasmid for HPV16 and linearized or circular plasmid for HPV45 was tested by using the chosen conditions for the two HPV types noted above. After the assembly reaction, samples were treated with nuclease and PsV production was evaluated by analyzing infection of HeLa cells. For both virus types, a clear dose dependent increase of infection with increase of DNA concentration was observed (FIG. 3B), although the increase in infectivity was not directly proportional to the increase in DNA and tended to plateau at the highest DNA amounts. 450 ng DNA per μg L1 protein was chosen for further studies, since PsV production was relatively efficient using these conditions and adding more plasmid DNA per μg L1 protein would be impractical in larger scale production.

Based on these findings, the inventors attempted production of highly concentrated stocks of papillomaviral vectors that could transduce a Luciferase and GFP-expressing plasmid (pCLucF), For HPV16, intact HPV16 VLPs were incubated in citrate buffer pH 5.2, no added NaCl, 0.02% Tween 80 and 450 ng/μg of linearized or circular Luc/GFP plasrnid for 48 h at 37° C. according to the results shown previously. After 48 h, the samples were nuclease treated for 3 h with 0.2% BAL-31 and 0.2% benzonase for 3 h at 37° C. in buffer containing 10 mM MgCl₂ and 0.5 M NaCl. Due to high dilutions required for standard disassembly leading to higher volumes, the disassembly protocol for HPV45 needed to be adjusted: HPV45 particles were disassembled in 200 mM NaCl, 20 mM Tris pH 8.2, 2 mM DTT, and 0.01% Tween80 for 6 h at 37° C. The inventors confirmed that particles disassembled under these conditions. After disassembly, HPV45 capsid proteins were reassembled in buffer containing 100 mM Tris pH 7.2, 150 mM NaCl, 10 mM CaCl2, 0.02% Tween80 and 450 ng/μg L1 protein of linearized or circular pCLucF according to the concentrations determined previously. For reassembly, the reaction was incubated for 48 h at 37° C. Nuclease treatment proceeded as for HPV16. These virus stocks were tittered in 293 TT and the particles were examined by electron microscopy.

Many of the HPV45 particles had larger than normal diameters, suggesting that these expanded particles were rather loosely assembled immature PsVs (FIG. 3C; “-GSSG”). To improve the quality of the particles, 5 mM oxidized L-Glutathione (“GSSG”) was added to the reassembly protocol, because GSSG has been shown to improve capsid maturation (Buck et 2005b; Journal of Virology 79:2839-46). The reassembly mix was incubated for 30 h without any GSSG to allow capsid assembly, and then 5 MM. GSSG was introduced for a further 15 h to allow for a “maturation” step. This addition led not only to tighter and more uniform particles but also to a higher titer stock when linear DNA was used (FIG. 3C; “-GSSG”). The addition of GSSG did not affect HPV16 titers or capsid morphology, which was not surprising because the capsids had not been disassembled. Nevertheless, the GSSG addition step was included during HPV16 production to maximize capsid stability for in vivo infection studies described below. To partially purify the PAT and increase their concentration, they were centrifuged over an OPTIPREP™ cushion. After centrifugation, the titers for both HPV16 and HPV45 were determined and compared with standard PsV stocks produced in 293TT cells (FIG. 3D). For HPV16, these “defined” papillomaviral vector production generated virus titers very similar to the titers obtained for standard HPV16 PsVs (FIG. 3D). For HPV45, the inventors obtained particles that were very similar or slightly higher in titer than the standard PsV production (FIG. 3D).

Given these encouraging results, the inventors decided to extend the PsV production methods to other PV types that showed high infectivity in preliminary experiments, specifically HPV58, 39, 59, 68, 26, and MusPV1 (see FIG. 2B). For all HPV types, disassembled VLPs were used with circular or linearized DNA, and for HPV58 and HPV26, intact VLPs were also used with linearized DNA, because it was not clear from the preliminary studies which conditions would produce the highest titers. When intact particles were used, the protocol described for HPV 16 was used and, when disassembled particles were used, the protocol described for HPV45 was applied. For HPV58, when linear DNA was used, high titers, very similar to standard PsV production, were obtained for VLPs that have been disassembled or intact. When circular DNA was used as the packaging substrate, titers were about 10-fold lower. For the α7 types, we only used disassembled particles. HPV59 PsV prepared by the “defined” method had either a similar titer or, in the case of linear DNA, even higher titers as standard preparations, but HPV39 titers for were about 10-100- fold less infectious. For HPV68, packaging of linear DNA resulted in infectivity similar to the standard produced PsV, while for circular DNA, infectivity was reduced about 10-fold. Production of in vitro HPV26 vectors also led to high titers. The highest titers were obtained for disassembled particles with linear DNA. In the case of circular DNA and disassembled particles, infectivity was reduced about 10-fold when comparted to standard PsV and about 100-fold lower for linear DNA with intact particles. For MusPV1, only disassembled particles were used and the pseudovirions produced were about 10-fold less infectious than standard PsV particles when linear DNA was used and about 100-fold less infectious particles with circular DNA. It is important to note that even in an instance were the titers of the defined-produced PsV were lower compared to the standard production method, titers were nevertheless quite high, at least 10⁸ infectious units/mg of L1 protein. (see FIG. 3D)

### Example 3: Characterization of Produced Pseudovirions

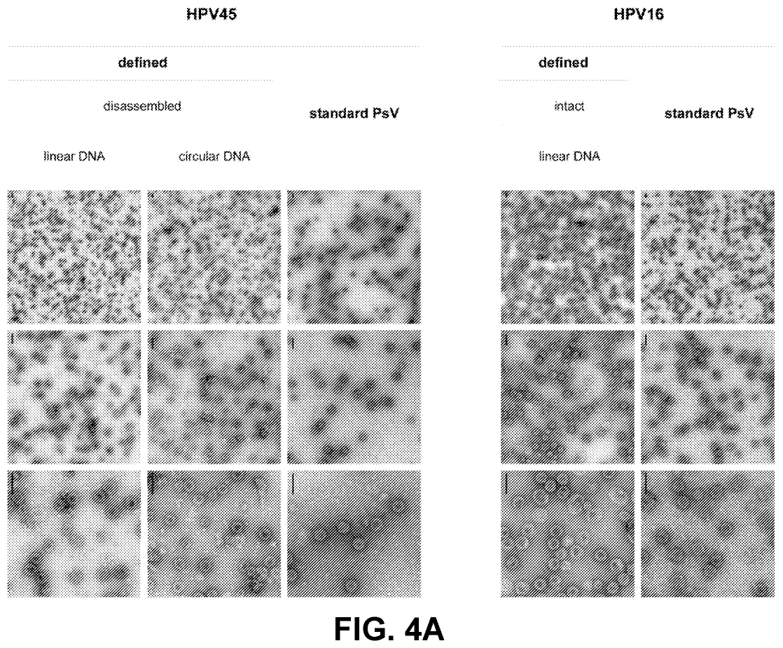

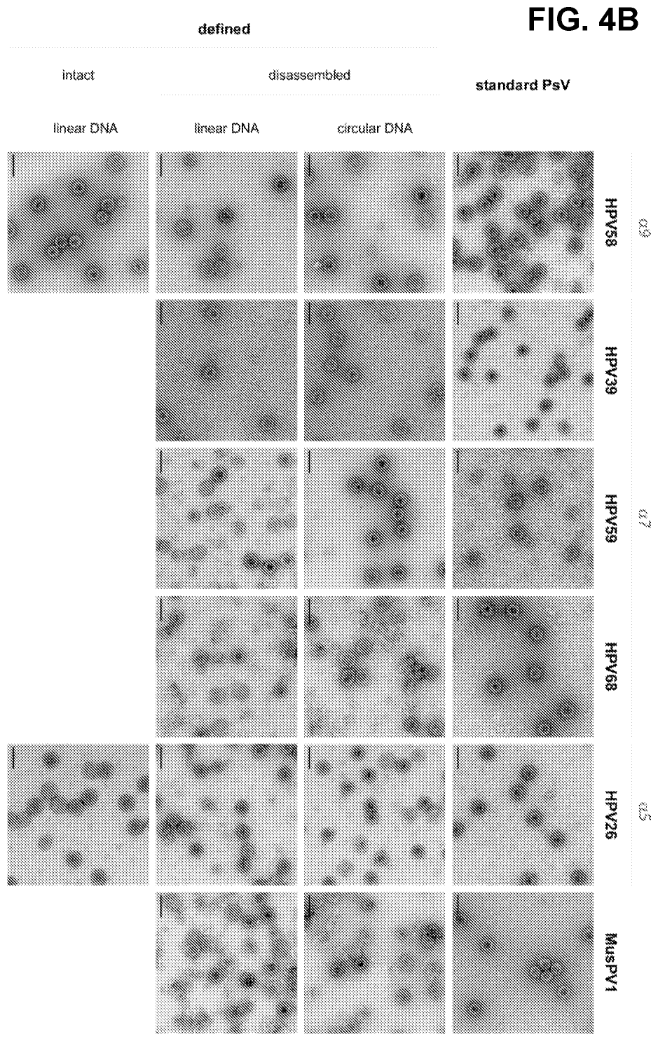

Having obtained high titer stocks of virus using these cell-free production protocols, it was important to further characterize the pseudovirion particles produced compared to standard PsVs produced in 293TT cells. Particle morphology was also analyzed along with sensitivity to inhibitors. HPV16 and HPV45 were used in assembly reactions based on the use of intact and disassembled capsid proteins, respectively. The particles produced in the final preparations were initially characterized by electron microscopy (FIG. 4A). For HPV16, most of the particles had a uniform VLP morphology, although some particles were slightly expanded (FIG. 4A). For HPV45, the morphology was more variable. Although there were many well-assembled particles, there were also a considerable number of expanded and partially-assembled particles in the preparation (FIG. 4A). Other high titer viral stocks were examined (FIG. 4B) and the results were similar to those obtained for HPV16 or HPV45. Generally, if the particles had been disassembled prior addition to the production reaction, then the resulting virus stock had more expanded and partially-assembled particles than stocks produced starting with intact particles (FIG. 4B).

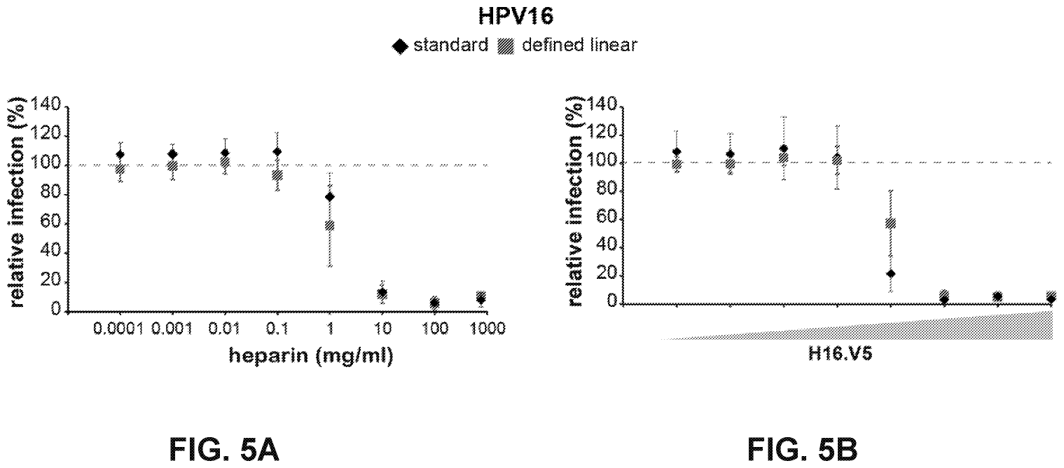

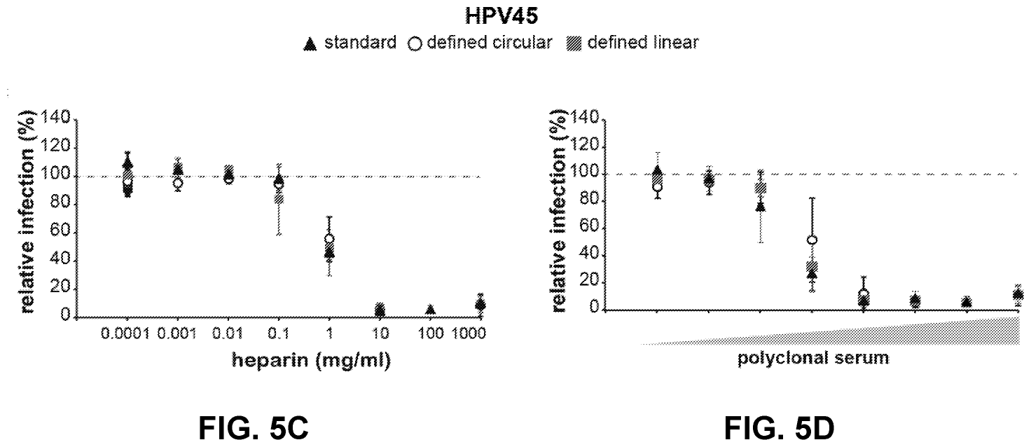

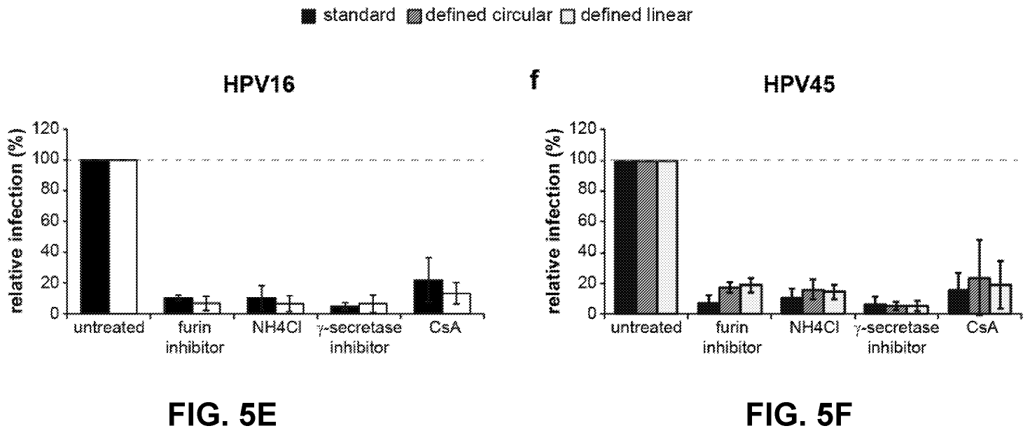

The susceptibility of these PsVs produced using the defined protocols of this disclosure, and those produced PA' by standard methods, to heparin, neutralizing antibodies, and entry inhibitors was compared (FIG. 5). HPV16 and HPV45 PsVs were incubated with serial dilutions of heparin for lh on ice and subsequently tested for 293TT cells infection. Defined reassembled HPV16, and both types of defined reassembled HPV45, were susceptible to heparin inhibition of infection to the same extent as the respective standard-produced PsVs (FIG. 5A and 5C). This suggests that the defined reassembled viruses also use heparan sulfate for attachment to immortalized cells in vitro. The same virus preparations were incubated with neutralizing antibodies and the capacity of the antibodies to inhibit 293TT infection was measured. For HPV16, a well-characterized monoclonal neutralizing antibody, H16.V5 was used, and, for HPV45, rabbit polyclonal serum raised against HPV45 L1 protein was used. HPV16 produced by the defined method of this disclosure was neutralized to the same extent as standard preps by H16.V5 (FIG. SB). In the case of HPV45, the polyclonal serum also neutralized infection of the defined reassembled particles to the same extent as standard PsVs (FIG. 5D)). These finding indicate that the major neutralizing epitopes on the standard PsVs are also present on the defined reassembled PsV.

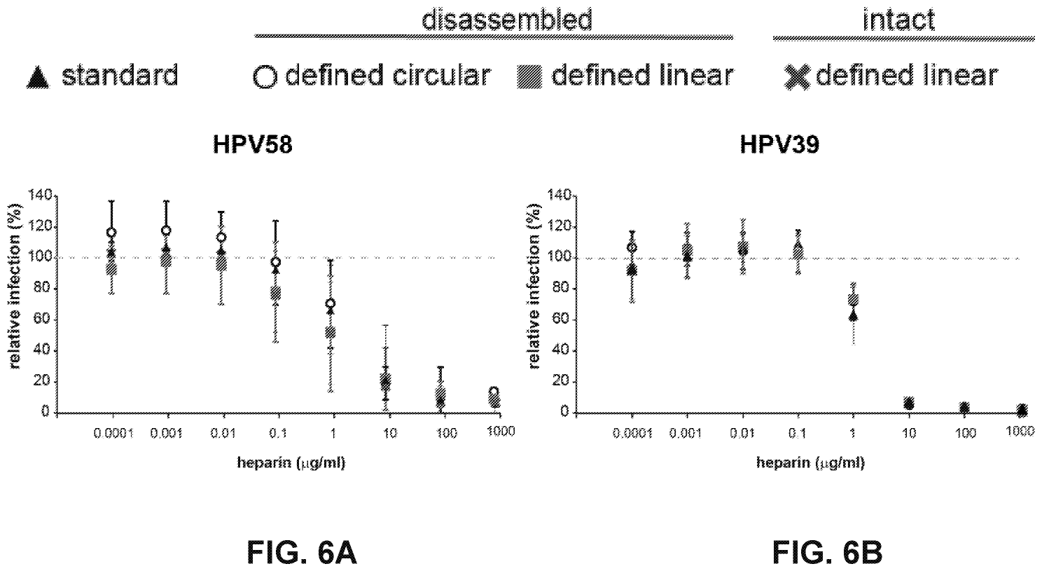

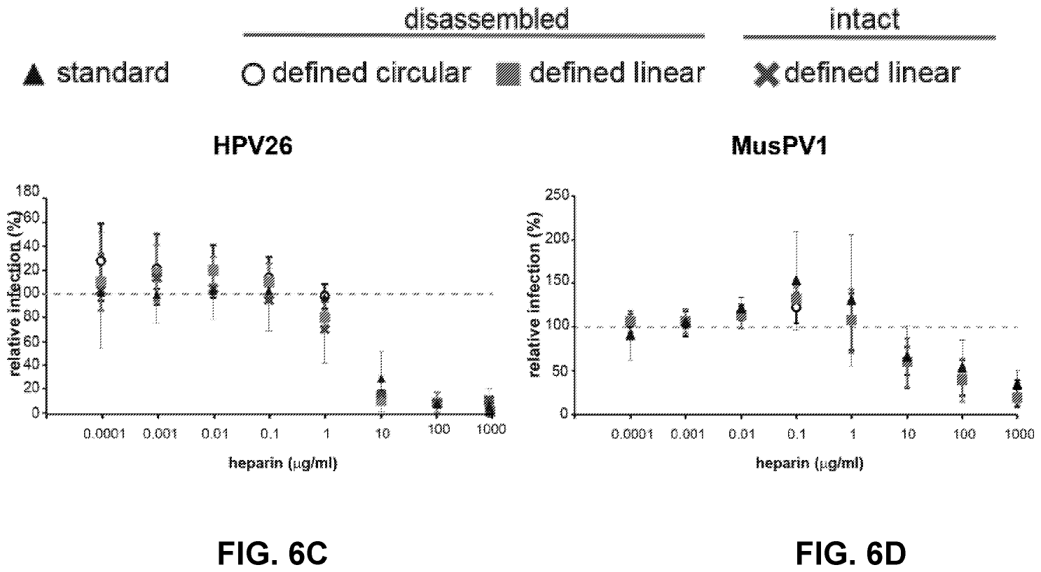

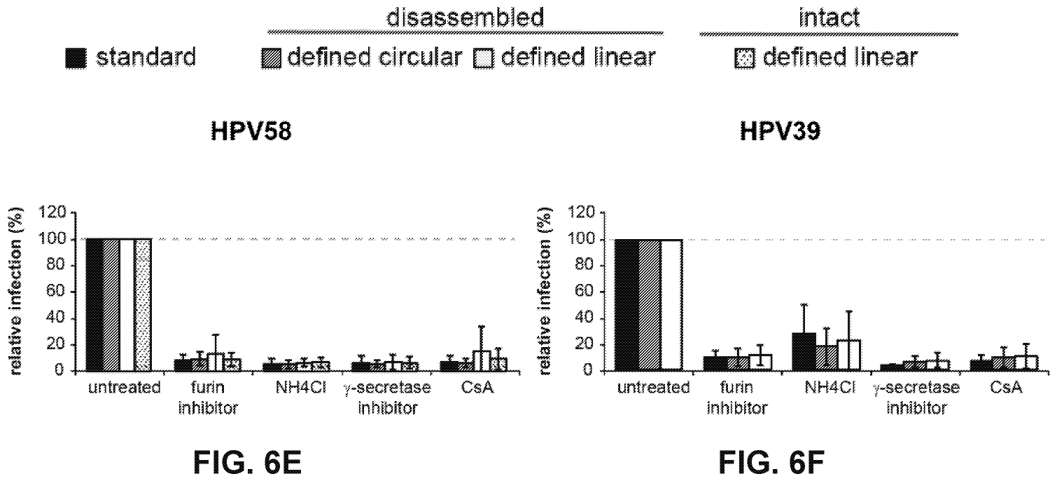

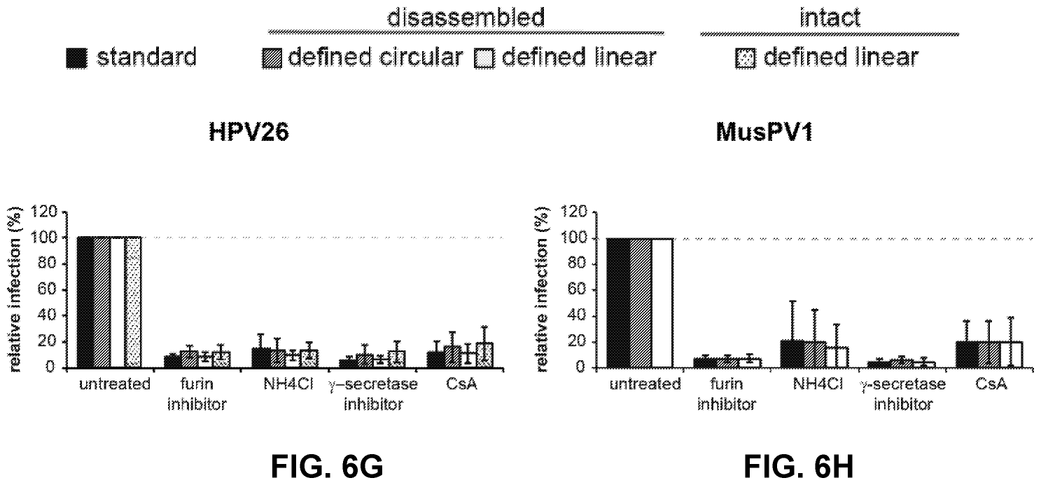

To evaluate sensitivity to inhibitors of cellular entry, 293TT cells were infected with the defined-produced or standard particles in the presence of a furin inhibitor (dec-RVKR-cmk), N1140, an inhibitor of y-secretase (compound XXI) or a cyclophilin inhibitor (cyclophilin A, CsA). These inhibitors are well known to inhibit HPV16 and HPV45 entry into cells. All inhibitors inhibited defined-produced and standard PsVs infection of 293TT to the same extent (FIGs. 5E and 5F), suggesting the two production systems generate PsVs that use the same entry pathway. The inventors confirmed that the inhibitors also inhibited infection into HeLa cells. These analyses were extended to HPV58, 39, 26, and MusPV1 (FIGS. 6A-H). For all these types, the same heparin and inhibitor sensitivity was observed for the defined reassembled particles and the standard produced PsVs as has been reported previously for standard produced PsVs (Day et al., 2015; Virology 481:79-94; Kwak et al., 2014; PloS One 9, e97232), Antibody neutralization could not be evaluated for all these types, due to the lack of the necessary antibody reagents. These types were also sensitive to furin inhibitor, NH₄Cl, compound XXI, and CsA to the same extent as standard virus.

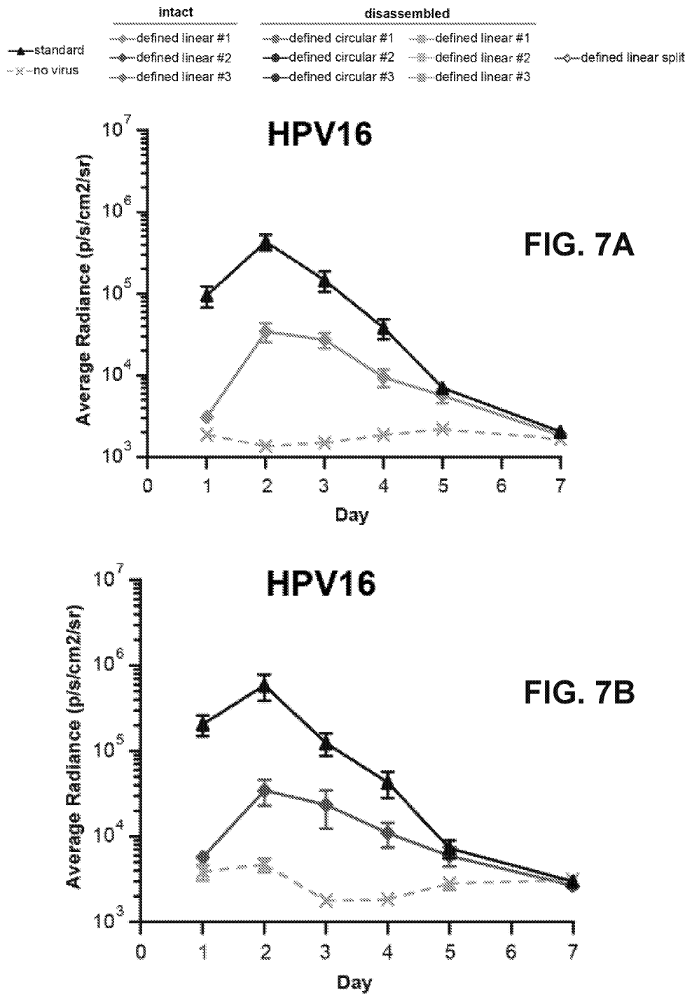

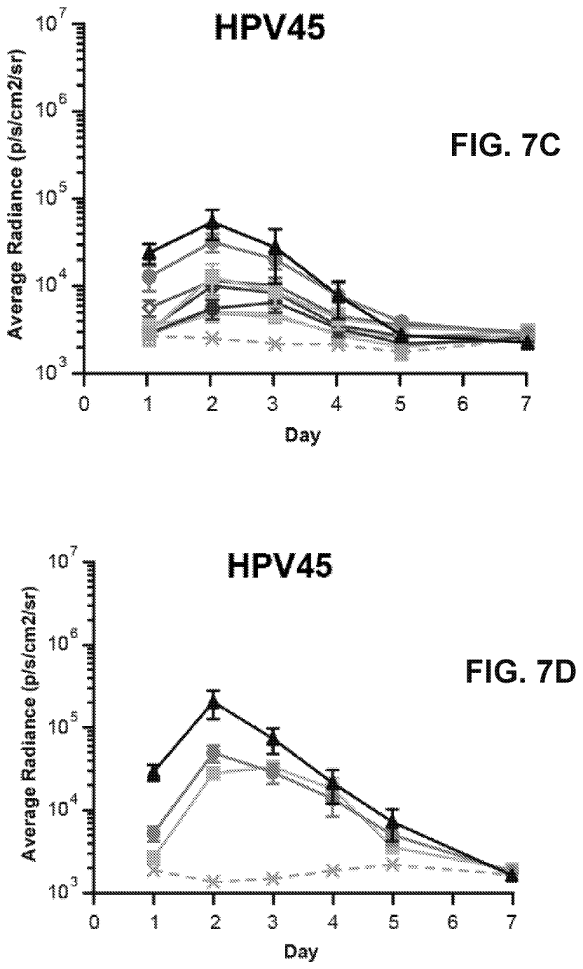

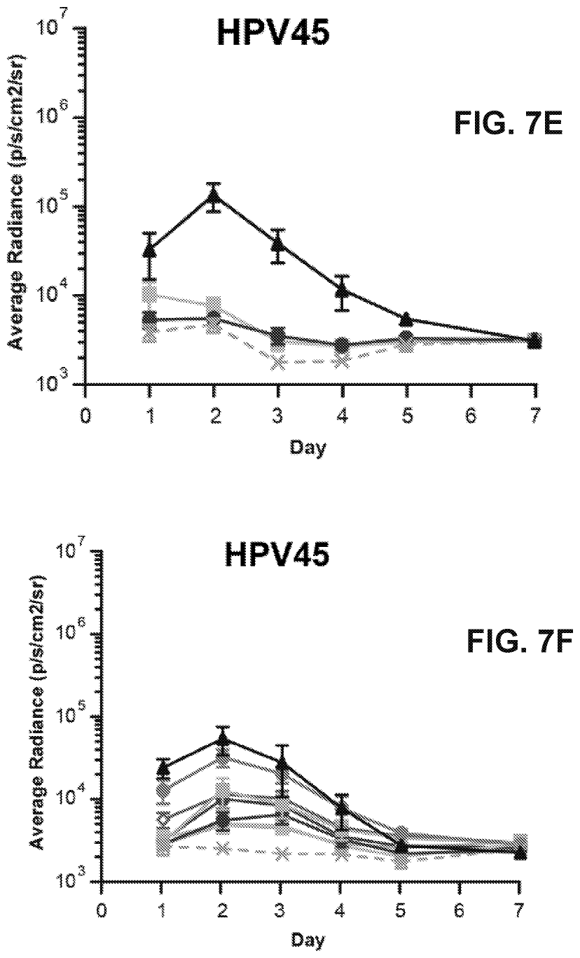

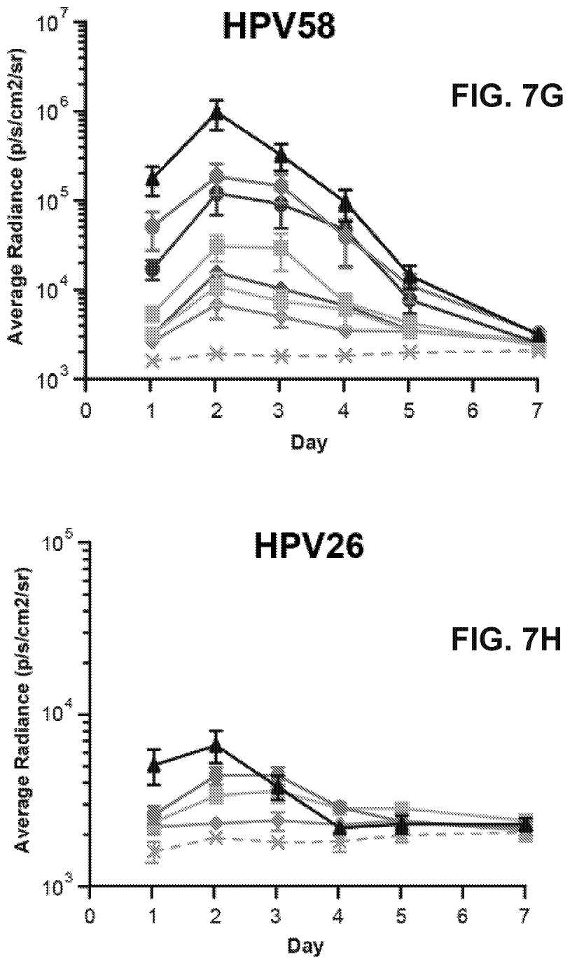

As a final characterization, in vivo infectivity of HPV16 and HPV45 was evaluated using our previously-described cervicovaginal model (Roberts et al., 2007; Nature Medicine 13:857-61). Mouse cervicovaginal epithelium was disrupted using nonoxynol-9, and the mice were infected with defined-generated HPV16 or HPV45 or the respective standards PsVs. The same number of 293TT infection units were applied in all cases. Infection of the vaginal tract was measured by luciferase expression on days one through seven post infection (FIGs.7A-7H). Infectivity of three different HPV16 and HPV45 was analyzed in in vitro-generated virus stocks in different experiments. Similarly, the three HPV45 stocks were also analyzed in the same experiment (FIG. 7F). The results were very consistent for the HPV16 preparations (FIGS. 7A-7C). The kinetics of infection by the in vitro-generated particles were similar to the standard PsV. A slight delay was observed on day 1 for the defined-generated particles. Cervicovaginal infectivity of the defined-generated particles was in general 2-5× lower when compared to the standard PsVs (FIGS. 7A-7C). For HPV45, the results were more variable (FIGS. 7D-7F). Although the kinetics of cevicovaginal infection were very similar for the define-produced and standard PsVs, one HPV45 preparation performed almost as well as the standard virus (FIGS. 7C and 7F, defined circular or linear #1), while the others had approximately 1.0-20× lower in vivo infectivity (FIGS. 7E,7F). We also tested HPV58 and HPV26 (FIGS. 7E,7H) on the cervicovaginal model. For HPV58, PsVs generated from disassembled VLPs and circular genome had the best in vivo to in vitro infection ratio, being about 5-8-fold less infectious than standard PsVs. PsVs generated from disassembled VLPs and linear DNA or intact VLPs and linear DNA were 30-100× less infectious in vivo than standard virus. For HPV26, the virus stocks were more dilute and so less infectious units were used per mouse, leading to an overall lower signal (FIG. 7H). Under these conditions, PsV generated from disassembled particles with either circular or linear DNA only had about 1.5-2-fold less infectivity than the standard preparations. PsVs generated from intact particles were about 3-fold less infectious than standard PsVs in the cervicovaginal challenge model. These results show that although the same in vitro infectious units were used per mouse, different PsVs, unexpectedly, have different infectivity efficiencies in vivo.

With the PsV that have encapsidated linear DNA, the inventors questioned whether the linearized plasmid re-circularizes during infection. To address this question, a further experiment was performed to determine the form of plasmid expressing the reporter gene in vivo. The Luciferase gene was split from its promoter by linearizing the plasmid at a restriction site between the two. Virus stocks were prepared using this linearized DNA (referred to as “defined linear split” virus) and mice were infected as described above. The Luciferase gene could be efficiently expressed only if the plasmid had re-circularized. Luciferase expression was observed in the vaginal tract of mice infected with HPV16 and HPV45 carrying this split plasmid (FIGS. 7C and 7F, “defined linear split”) with kinetics and level of expression similar to the other defined-generated PsVs. These results indicated that most of the transduced plasmid is in a circular conformation, which might be less prone to integrate into the host cell DNA than linear forms.

### Example 4: HPV16 and HPV45 Pseudovirion Packaging of mRNA

As has been discussed herein, one goal of the invention is the use of VLPs produced using methods of the invention, to deliver toxic genes to cancer cells. However, DNA is prone to integrate into the host genome (Chancham and Hughes, 2001; Chen et al., 2001), which could result in unforeseen problems. The use of mRNA would be safer for gene delivery, due to the absence of integration and relatively shorter half-life. Thus, studies were conducted to investigate the ability of PV capsid proteins to package mRNA.

VLPs made from HPV16 and HPV45 capsid proteins were chosen for preliminary analysis. mRNA encoding enhanced green-fluorescent protein (eGFP) was chosen for packaging, due to its easy availability and the simplicity of the readout. Initially, the best reassembly conditions found for linear DNA packaging were used for packaging mRNA. That is pH 5.2 for intact particles and pH 7.2, 150 mM NaCl, 10 mM CaCl2, 0.02% Tween80, for disassembled particles. Different amounts of mRNA (including 16 ng, 160 ng and 1600 ng mRNA per microgram of L1 protein, corresponding to 1:1, 1:10, 1:100 capsid to mRNA ratio). Under these conditions, there was no infection for HPV16 with any of the mRNA concentrations used (Data not shown). For HPV45, there was mRNA concentration-dependent infection. (Data not shown) Infection was better for disassembled particles but also present for intact (not disassembled) particles. Infection was best at the highest concentration of mRNA used. Infectivity could be inhibited by HPV45 L1 neutralizing sera, indicating infection was driven by HPV45 capsid (Data not shown).

### Example 5: Packaging of mRNA by Expanded Papillomavirus Pseudovirion Types

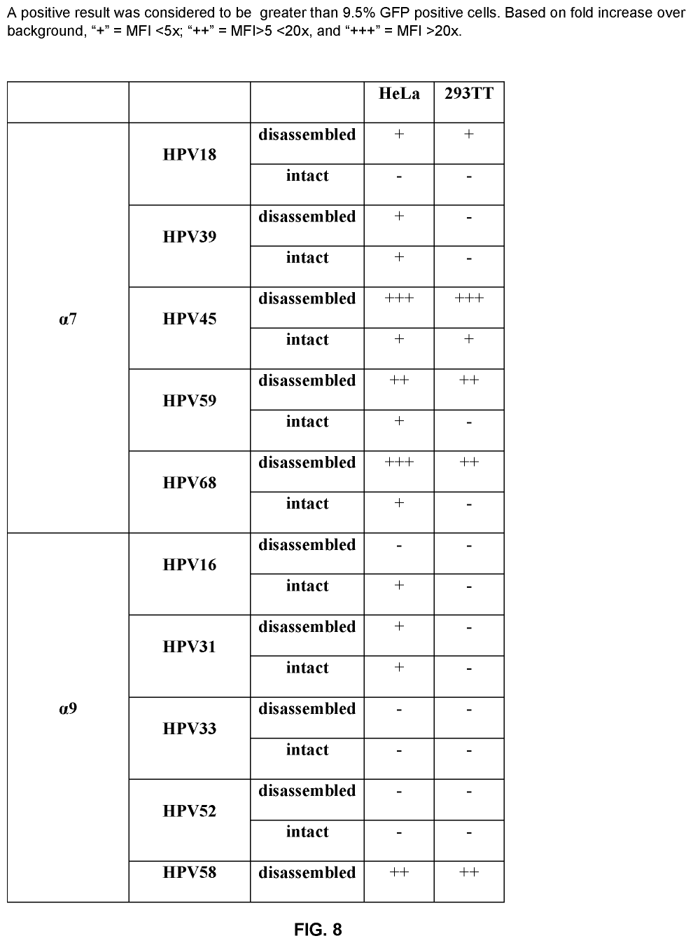

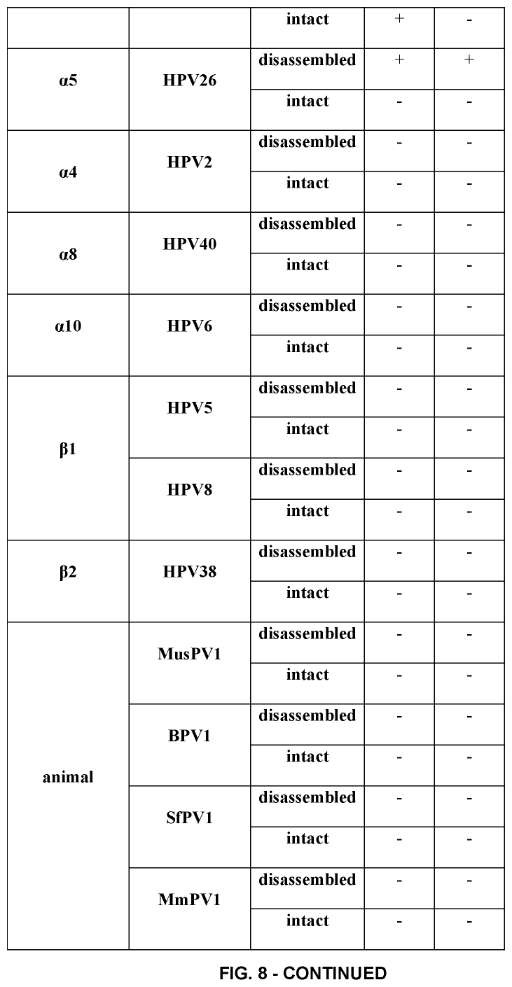

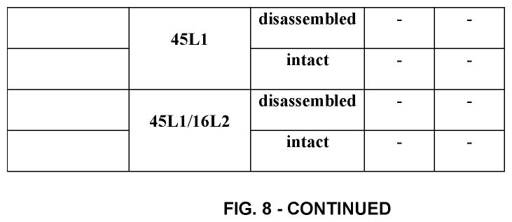

Additional studies were conducted to explore the number of PV VLPs capable of packaging sGFP-encoding mRNA. The efficiency of packaging was tested using both disassembled and intact VLPs from HPV types 16, 31, 33, 52 and 58 (α9 types), HPV types 18, 39, 45, 59, 68 (α7 type), HPV2 (α4 type), HPV26 (α5 types), HPV 6 (α10 type), HPV 5 and 8 (β1 type), HPV38 (β2 types) and the animal types BPV1, MusPV1, MmPV1 and SfPV1. Disassembly of the VLPs was performed in low salt and DTT using the procedure for DNA described by Cerqueira et al., 2017. The disassembled L1/L2 capsid proteins, or the intact VLPs, were mixed with eGFP mRNA a ratio of 1:100 (capsid to eGFP mRNA), in 150 mM NaCl at pH 7.2. The mixture was incubated for about 20 h, after which it was treated with RNase to digest all unpackaged RNA. The resulting VLPs were then used to infect HeLa or 293TT cells, and GFP expression in the cells was measured by flow cytometry at 72 hour post-infection (p.i.). To score for true infection and avoid false positives, ‘infectivity’ was defined as only those samples where more than 10% of cells exhibited green fluorescence. To distinguish between high and low levels of infection, different levels of fluorescence were defined relative to the mean fluorescence intensity (MFI); low infection (+) was defined as a MFI<5-fold background; medium infection (++) was defined as a MFI<20-fold background; and high infection (+++) was defined as a MFI>20-fold above background. The results of this experiment are shown in FIG. 8.

As can be seen from the Table in FIG. 8, in HeLa cells, VLPs made from the α9 clade viruses HPV16, HPV31 and HPV58, had low infectivity when used as intact particles before adding the mRNA. HPV31 VLPs also had a low infectivity when disassembled, but HPV58 VLPs had an intermediate infectivity when disassembled before adding the mRNA. HPV31 and HPV52 VLPs had no infectivity. For VLPs made from α7 clade virus proteins, the results are very similar for all types tested. Infectivity was very low or even absent if the virus were not disassembled (i.e., mRNA added to intact particles), but infectivity was medium or high when particles were disassembled prior to contact with mRNA. The only exception in the α7 group was HPV39, for which infectivity was low regardless of whether the particles were disassembled or intact prior to contact with the mRNA. The single α5 clade member tested, HPV26, also showed infection when used in its disassembled form, but not in the intact form. For all other clades tested, (i.e., α4, α8, α10, β1, β2 and the animal types), no infectivity was observed. These results indicate that some PV types can efficiently package mRNA, which is surprising since PVs have only been reported to package DNA.

Previous work has shown that the L2 protein is required for PV VLP delivery of DNA. Thus, experiments were performed to determine if the L2 protein is also required for infectivity of PV VLPs carrying the packaged mRNA. This analysis was performed using HPV45 L1-only VLPs (FIG. 8). Packaging of eGFP mRNA, and contact of the VLPs with cells was performed as described above. The results showed that L1-only VLPs failed to infect the cells, suggesting that L2 is required for infection and/or packaging of mRNA.

In a further experiment, the disparity between HPV16 and HPV45 was utilized to assess the infectivity of an HPV45L1 HPV16L2 chimera, which known to be infectious when carrying a DNA cargo (Day et al., 2015b). The chimeric pseudovirions were infectious (see construct labeled 45L1/16L2 in FIG. 8), indicating that heterologous combinations of L1 and L2 capsids can also transduce mRNAs. Surprisingly, the inverse capsid configuration (i.e., HPV16 L1 HPV45 L2) did not produce infectious particles. Similar results were obtained using 293TT cells. Generally, the eGFP signal in 293TT was lower than in HeLa cells which may explain some of the virus types exhibiting lower infectivity. The differences in signal expression are probably linked to the different mRNA expression efficiencies in the two cell lines

These results indicate that PVs members of the α7, α9 and α5 can transduce mRNA. Transducing titers were higher when disassembled particles were used, and best for the α7 clade.

### Example 6 Inhibition of PV VLP Mediated Delivery of mRNA

Given the surprising ability of PV VLPs to transduce mRNA into cells, further experiments were conducted to determine if transduction of cells by mRNA-containing PV VLPs is sensitive to the same inhibitors that affect transduction by DNA-containing PV VLPs. These experiments were conducted using VLPs made from HPV types 18, 39, 45, 59, 68, all of which are PV α7 members and which previously showed high infectivity, and HPV types 58 and 26. In all instances, particles were disassembled prior to the reassembly reaction since under these conditions we obtained higher infection. For HPV types 58, 18 and 45 it was also determined if an anti-L1 neutralizing sera could inhibit infection. All of the tested VLPs were inhibited by the corresponding neutralizing sera indicating that mRNA expression is dependent on HPV infection (FIG. 9A). Heparin, which is known to inhibit PV attachment to cell surface heparan sulfate proteoglycans (HSPGs) required for infection (Cerqueira et al., 2013; Combita et al., 2001; Day et al., 2015a; Giroglou et al., 2001; Johnson et al., 2009; Joyce et al., 1999; Kines et al., 2009; Kwak et al., 2014; Shafti-Keramat et al., 2003), was then tested for its ability to inhibit by the VLPs. The results, shown in FIG. 9A, revealed that heparin inhibited infectivity of all virus types tested, indicating viral attachment for virus containing mRNA is dependent on cell surface HSPGs.

Additional inhibitors were also tested for their ability to inhibit the VLP entry process. These inhibitors were NH4Cl, an acidification inhibitor (Dabydeen and Meneses, 2009; Schelhaas et al., 2012), γ-secretase inhibitor (γ-sec inh) (Huang et al., 2010; Karanam et al., 2010; Kwak et al., 2014), and cyclosporine A (CsA) (Bienkowska-Haba et al., 2009). The results, shown in FIG. 9A, showed that all compounds of the compounds inhibited all tested HPV infections, indicating that mRNA-containing VLPs entry occurs through the same mechanism as cell-derived HPV PsVs.

### Example 7. Nuclear Trafficking of mRNA Delivered by PV VLPs

Because mRNA is translated in the cytoplasm, it was not clear if transit through the nucleus was required for infection, as has been reported for cell-derived DNA-containing PsVs (Aydin et al., 2014; Pyeon et al., 2009). This question was examined using the cell cycle inhibitor aphidicolin (Aph), which interferes with HPV infection by inhibiting exit from the trans-goligi network (Aydin et al., 2014; Pyeon et al., 2009), a prerequisite for nuclear entry. The results of this study (shown in FIG. 9A) demonstrated that aphidicolin inhibited transduction of mRNA-containing VLPs, indicating that mRNA expression and infection required virus passage through the nucleus before being exported to the cytoplasm.

In summary, these results suggest that the mRNA-containing PV VLPs follows the same pathway as HPVs containing DNA.

The results obtained with aphidicolin indicated that the eGFP-encoding mRNA traffics through the nucleus before it is exported to the cytoplasm, where it is expressed. To better study the mechanism of mRNA delivery, the effect of Leptomycin B, an inhibitor of CRM1/Exportin 1 (Kudo et al., 1999) that inhibits the export of most mRNAs from the nucleus by inhibiting export of ribonucleoproteins (Bai et al., 2006; Brennan et al., 2000; Jang et al., 2003; Kaida et al., 2007; Wolff et al., 1997), was tested. The results, which are illustrated in FIG. 9B, showed that Leptomycin B inhibited HPV45 VLP-mediated mRNA transduction in a dose dependent manner. Infection with DNA PsVs was also inhibited by leptomycin B (FIG. 9B), which further confirms that the PV VLP-delivered mRNA is trafficked into the nucleus in a manner similar to the PV DNA cargo, and thus requires exportation into the cytoplasm for protein expression. Furthermore, nuclear export of the delivered mRNA is dependent on CRM1/Exportin 1. To control that the inhibitor was not toxic for the cells, which would affect infectivity, control cells were exposed to Vaccinia Virus (FIG. 9C). The replication cycle of vaccinia virus does not depend on the nucleus and thus, leptomycin should not affect its entry and infection (Condit et al., 2006). As expected, leptomycin did not inhibit vaccinia virus infection, indicating that the inhibitor is not affecting VLP infectivity per se.

### Example 8. Packaging of mRNA Encoding Toxic Proteins by PV VLPs

As has been stated above, one goal of the invention is to use PV VLPS to deliver mRNA encoding toxins into cancer cells. Such VLPs cannot be produced using standard method of VLP production, since expression of the toxin in the producer cells would kill the cell before sufficient numbers of VLPs could be produced. Thus, the ability of PV VLPs to package toxin-encoding mRNA in vitro, and deliver such them into cells, was tested.

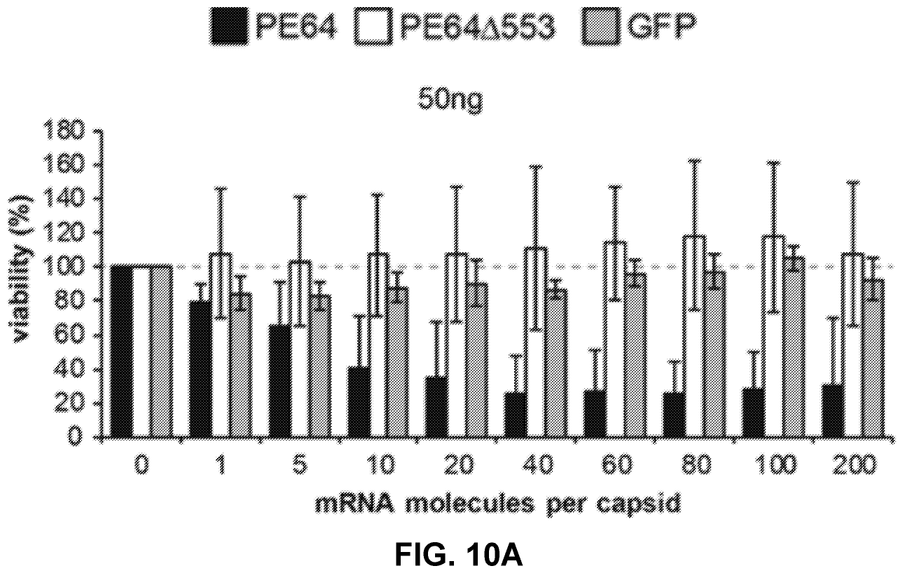

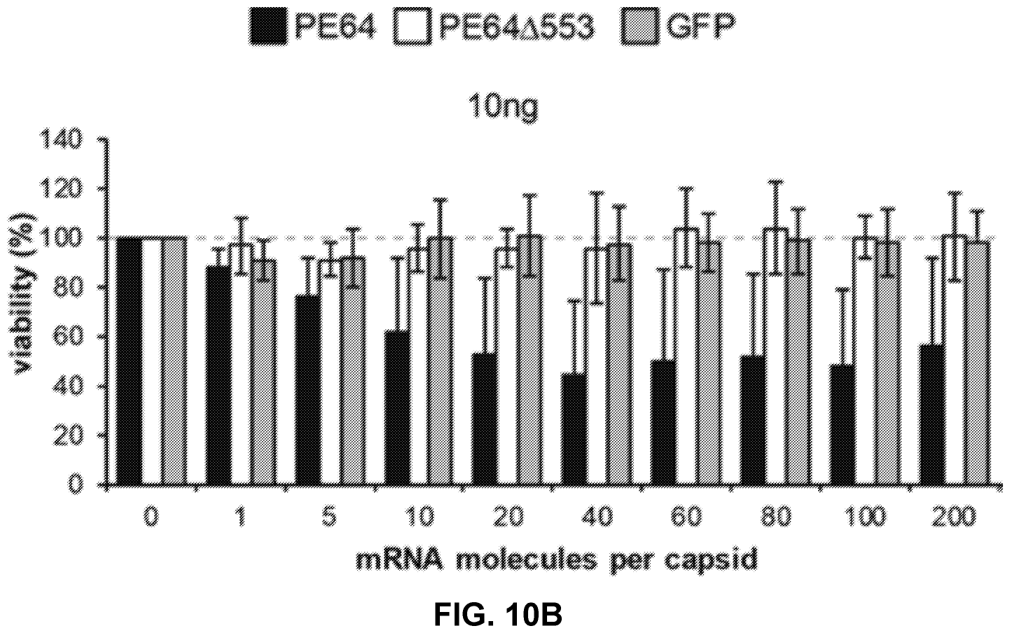

The toxin used in these studies was Pseudomonas exotoxin A (PE), PE64 (FitzGerald et al., 1998; Hertle et al., 2001). Both an active form (PE64), and a mutated, enzymatically inactive form (PE644553) were used. L1 and L2 proteins from HPV45 were used to produce the VLPS, since this PV type was one of the most efficient types for mRNA transduction (see FIG. 8). Packaging of the mRNA into VLPs was performed as described above. However, several ratios of capsid to mRNA (i.e., 1, 5, 10, 20, 40, 60, 80, 100 and 200 mRNA molecules for each fully formed HPV capsid) were tried. After packaging of the PE-encoding mRNAs, HeLa cells were infected using the same amount of L1 for all samples, and cell death determined by measuring cell viability using the XTT assay. The results are shown in FIG. 10A & 10B. VLPs containing PE64-encoding mRNAs induced cell death in a mRNA concentration dependent manner. A plateau for cell death was reached at 20 molecules per capsid, after which no increase in cell death was observed. As expected transduction of cells with VLPs containing mRNA encoding the inactive form PE644553, had no effect on cell viability, with equivalent toxicity to the control eGFP mRNA PV. Together these results show that cell death can be induced by delivery of a PV VLP comprising an mRNA encoding a toxin. A 1:60 ratio of mRNA/capsid using high titer stocks of HPV45 packaging PE64, PE644553 and GFP mRNA was chosen for subsequent experiments.

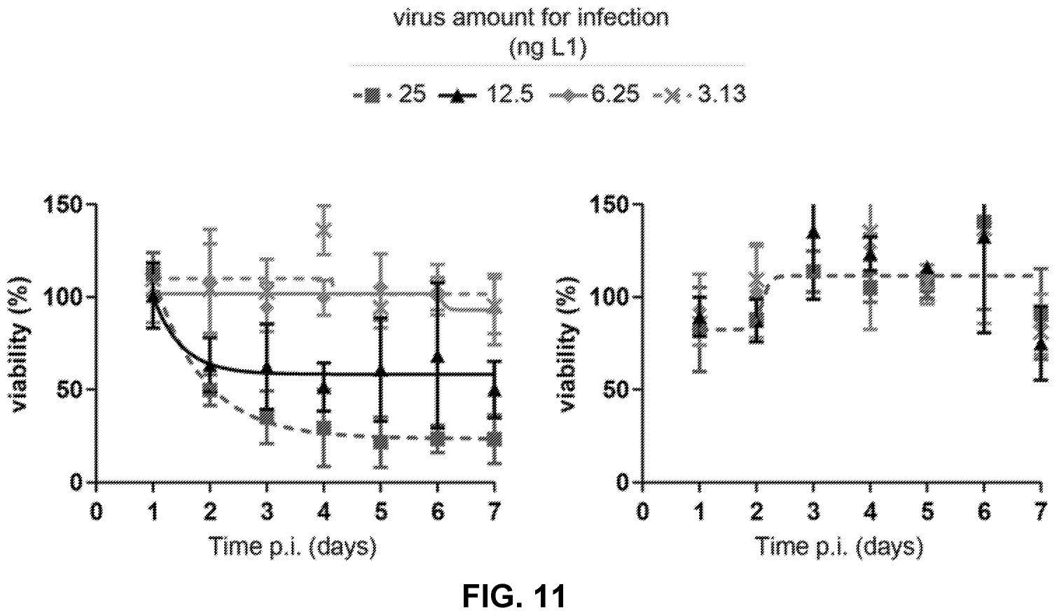

Next, a time course study of PE64-induced cell death was conducted. VLPs comprising mRNA encoding PE64, PE644553 or GFP, were produced as described above. However, for these experiments, a 1:60 ratio of mRNA/HPV45 capsid protein was used. Following packaging of the mRNAs, cells were infected with different amounts of VLPs, and cell viability was measured daily for seven days post infection. The results, which are shown in FIG. 11, demonstrate that cell death was dependent on the virus dose. At 24 h p.i., no dead cells over background were detected. Cell death was detected beginning at 48 h p.i. and plateaued between 72-96 h p.i. (FIG. 11—Left Panel). This time course was consistent with that determined previously for DNA transduction of HPV PsVs, where the virus required approximately 24 h to reach the nucleus and commence genome expression. Infection with the inactive mutant PE644553 had no effect on viability (FIG. 11—Right Panel).

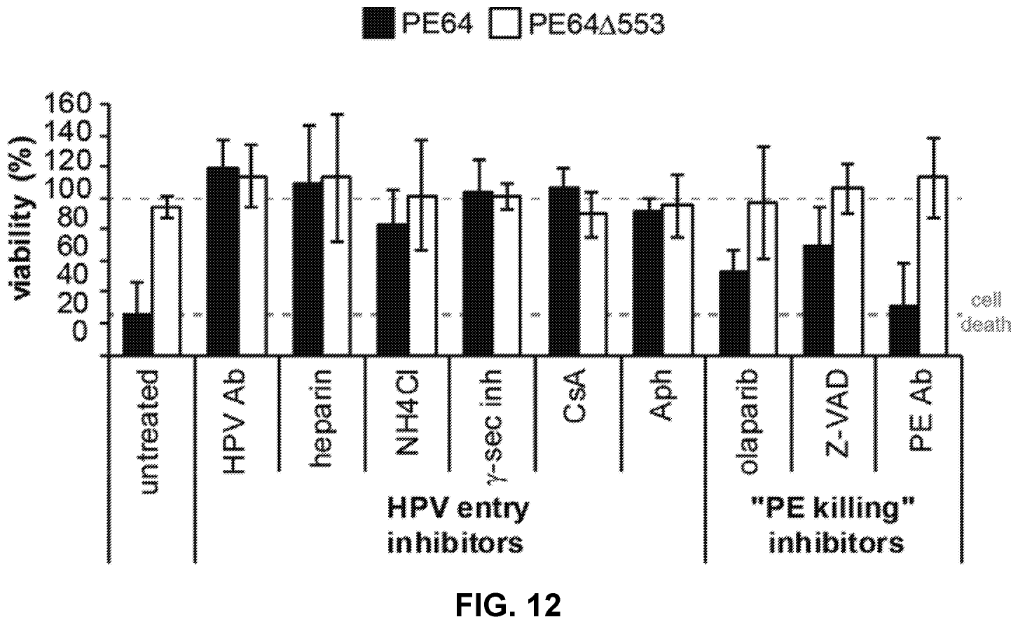

To confirm that cell death was driven by HPV45 expressing PE-encoding mRNA, experiments were performed using both HPV entry inhibitors and PE inhibitors. For HPV inhibition, an HPV45 L1-neutralizing serum, heparin, NH4Cl, cyclosporine A, and aphidicolin were used. As shown in FIG. 12, each of these inhibitors blocked cell death, confirming that cell death was mediated by infection with the mRNA containing VLPs. To prevent killing by PE, the apoptosis inhibitor, Z-VAD-fmk, was used, as PE-induced cell death is known to be by apoptosis (Sharma and FitzGerald, 2010). In addition, the PARP inhibitor, olaparib, which is known to inhibit the ADP-ribosylating function of the PE toxin (Antignani et al., 2016), was tested. Both inhibitors blocked cell death (FIG. 12), indicating that the enzyme activity of PE was responsible for the death. To monitor for toxin secretion and killing of neighboring cells, an anti-PE neutralizing antibody, which would neutralize extracellular toxin, was also tested. As expected, this treatment had no effect, since the toxin was only expressed intracellularly (FIG. 12). These results demonstrate that the induced cell death resulted from production of the toxin from the MRNA carried into the cell by the PV VLPs.

### Example 9. VLPS from Various HPV Types are capable Of Transducing Toxin-Encoding mRNA

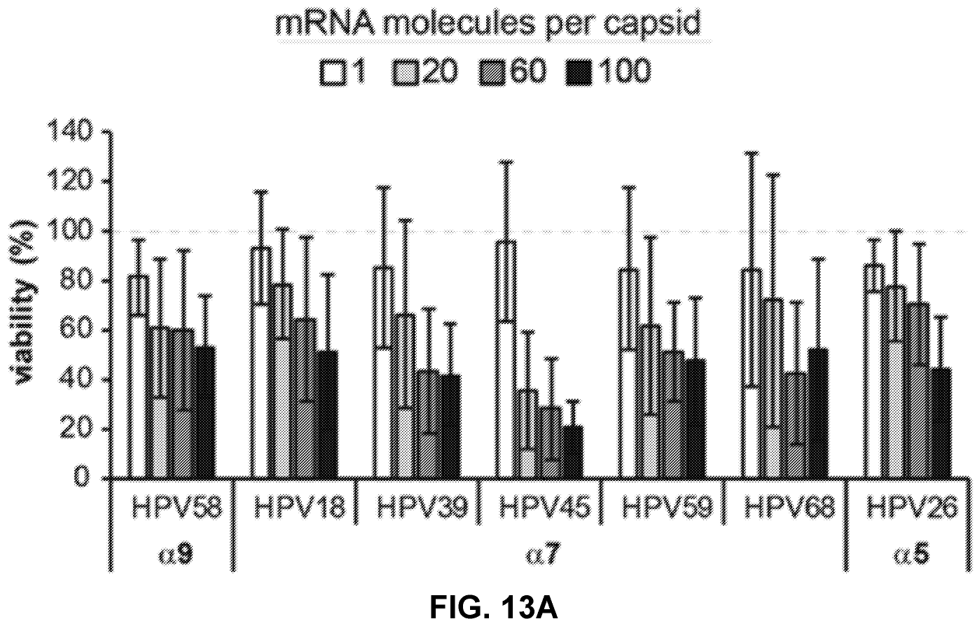

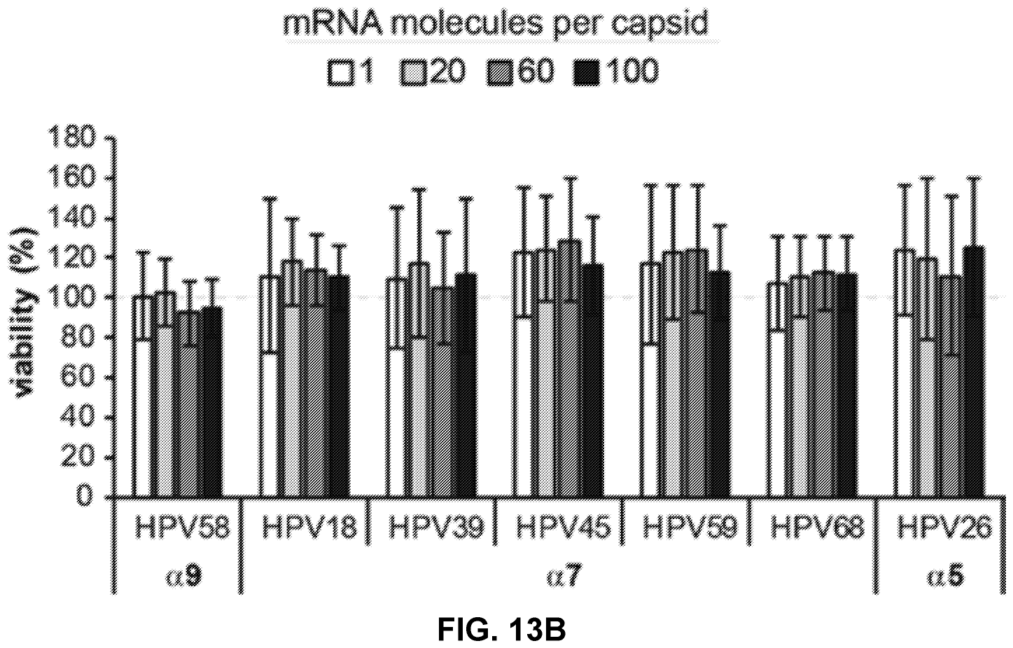

Several other HPV types were tested to determine if their capsid proteins could also package PE64 toxin mRNA, and deliver such mRNA into cells, thereby inducing cell death. Specifically, mRNA encoding the PE64 toxin was packaged using HPV types 58, 18, 39, 59, 68, 26, at mRNA molecule to capsid ratios of 1:1, 20:1, 60:1 and 100:1. Packaging using the same PV types and ratios was also performed using the inactive mutant. As shown in FIG. 13A, all viruses comprising mRNA encoding PE64 induced cell death in a mRNA dose dependent manner. However, none of the additional types were as effective as VLPs made from HPV45 in inducing cell death. VLPs comprising mRNA encoding the inactive mutant PE64Δ4553 had no effect on viability (FIG. 13B). These results indicate that several virus types could be utilized to deliver toxin mRNA.

### Example 10. PsVs Carrying Toxin-Encoding Mrna Can Kill Cancer Cells

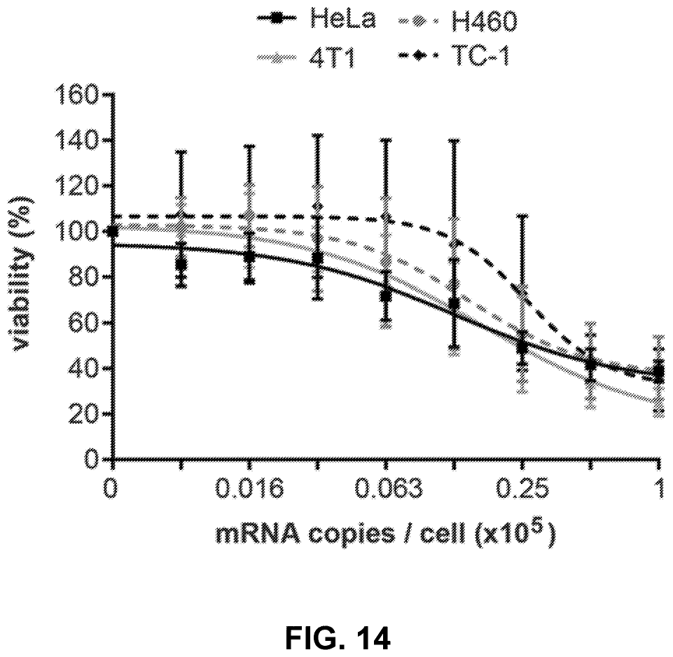

To confirm that PE could be used to induce cell death in cancer cells, the potency of PE64 in several different cancer cell lines, including human and mouse types, was tested. PE64-encodig mRNA was packaged using capsid proteins from HPV45, as described above. The resulting VLPS were then used to infect several cell lines, including H460 (human long cancer cells) and the mouse mammary gland cancer 4T1, and cell viability measured at 72 hours post-infection using the XTT assay. The results, which are shown in FIG. 14, demonstrate that infection with the VLP resulted in a decrease in cell viability in all cell lines tested.

The present disclosure is not to be limited in scope by the specific embodiments described herein which are intended as single illustrations of individual aspects of this disclosure, and functionally equivalent methods and components are within the scope of this disclosure. Indeed, various modifications of this disclosure, in addition to those shown and described herein will become apparent to those skilled in the art from the foregoing description and accompanying drawings. Such modifications are intended to fall within the scope of the claims.

1-13. (canceled)

14. A method of producing a papillomavirus pseudovirus comprising a therapeutic nucleic acid molecule, the method comprising:
a. mixing a virus like particle (VLP) comprising papillomavirus L1 and L2 proteins, with the therapeutic nucleic acid molecule, wherein the mixture lacks cellular factors; and,
b. incubating the mixture under conditions such that the VLP encapsidates the therapeutic nucleic acid molecule, thereby producing a papillomavirus pseudovirus comprising the therapeutic nucleic acid molecule.

15. The method of claim 1, wherein the papillomavirus pseudovirus comprising the therapeutic nucleic acid molecule has an infectivity to particle ratio of at least 1×10⁸ i.u./mg L1 protein.

16. The method of claim 1, wherein the mixture comprises less than 600 mM NaCl.

17. The method of claim 1, wherein the pH of the mixture is in the range of from 5.2 to less than 8.2.

18. The method of claim 1, wherein the step of mixing comprise contacting the VLP with at least 50 ng of the therapeutic nucleic acid molecule.

19. The method of claim 1, wherein the L1 and L2 proteins are from a HPV type selected from the group consisting of α4, α5, α7, α8, α9, α10, β1 and β1.

20. The method of claim 1, wherein prior to the step of incubating the mixture to encapsidate the therapeutic nucleic acid molecule, the VLP is disassembled.

21. The method of claim 20, wherein disassembly of the VLP comprises subjecting the VLP to conditions comprising less than 200 mM NaCl.

22. The method of claim 21, wherein disassembly comprises a reducing agent or a detergent.

23. The method of claim 21, wherein disassembly of the VLP comprises a pH in the range of about 7.2 to about 8.2.

24. A papillomavirus pseudovirus that comprises a therapeutic nucleic acid molecule, produced according a method comprising:
a. mixing a virus like particle (VLP) comprising papillomavirus L1 and L2 proteins, with the therapeutic nucleic acid molecule, wherein the mixture lacks cellular factors; and,
b. incubating the mixture under conditions such that the VLP encapsidates the therapeutic nucleic acid molecule, thereby producing a papillomavirus pseudovirus comprising a therapeutic nucleic acid molecule.

25. The papillomavirus pseudovirus of claim 24, wherein the papillomavirus pseudovirus has an infectivity to particle ratio of at least 1×10⁸ i.u./mg L1 protein.

26. The papillomavirus pseudovirus of claim 24, wherein the mixture comprises less than 600 mM NaCl.

27. The papillomavirus pseudovirus of claim 24, wherein the pH of the mixture is in the range of from 5.2 to less than 8.2.

28. The papillomavirus pseudovirus of claim 24, wherein the L1 and L2 proteins are from a HPV type selected from the group consisting of α4, α5, α7, α8, α9, α10, β1

29. The papillomavirus pseudovirus of claim 24, wherein prior to the step of incubating the mixture to encapsidate the therapeutic nucleic acid molecule, the VLP is disassembled.

30. The papillomavirus pseudovirus of claim 29, wherein disassembly of the VLP comprises subjecting the VLP to conditions comprising less than 200 mM NaCl.

31. The papillomavirus pseudovirus of claim 29, wherein disassembly comprises a reducing agent or a detergent.

32. The papillomavirus pseudovirus of claim 29, wherein disassembly of the VLP comprises a pH in the range of about 7.2 to about 8.2.

33. A method of delivering a therapeutic nucleic acid molecule into a cell, comprising contacting the cell with the papillomavirus pseudovirus of claim 24.


SEQUENCE LISTING (9 sequences)

SEQ ID NO: 1 - DNA, 18 bp, Artificial Sequence
  Synthetic primer
aaagcgctggagcgaatg

SEQ ID NO: 2 - DNA, 21 bp, Artificial Sequence
  Synthetic primer
gatgaccgtgggtttgatgtc

SEQ ID NO: 3 - DNA, 17 bp, Artificial Sequence
  Synthetic primer
aaggtgttgtgcctgct

SEQ ID NO: 4 - DNA, 20 bp, Artificial Sequence
  Synthetic primer
ctgaacagcatgggcatcag

SEQ ID NO: 5 - DNA, 19 bp, Artificial Sequence
  Synthetic primer
gggtggcaaatgggaagtc

SEQ ID NO: 6 - DNA, 15 bp, Artificial Sequence
  Synthetic primer
cagcccaccgtcgta

SEQ ID NO: 7 - DNA, 20 bp, Artificial Sequence
  Synthetic primer
cggcatcaaggtgaacttca

SEQ ID NO: 8 - DNA, 20 bp, Artificial Sequence
  Synthetic primer
accatgtgatcgcgcttctc

SEQ ID NO: 9 - DNA, 17 bp, Artificial Sequence
  Synthetic primer
ccactaccagcagaaca